(19) United States
(12) Patent Application Publication
(10) Pub. No.: US 2007/0232582 A1
(43) Pub. Date: Oct. 4, 2007

(54) 4-substituted or unsubtituted-7-hydro-1,4-thiazepine-7-[bicyclic or tricyclic heteroaryl] substituted-3,6-dicarboxylic acid derivatives as beta-lactamase inhibitors

(75) Inventors: Tarek Suhayl Mansour, New City, NY (US); Aranapakam Mudumbai Venkatesan, Rego Park, NY (US)

Correspondence Address: WYETH;PATENT LAW GROUP, 5 GIRALDA FARMS, MADISON, NJ, 07940, US

(73) Assignee: Wyeth Holdings Corporation, Madison, NJ (US)

(21) Appl. No.: 11/807,397

(22) Filed: May 25, 2007

Related U.S. Application Data
(63) Continuation of application No. 10/834,301, filed on Apr. 28, 2004, now Pat. No. 7,229,983.

(51) Int. Cl. A61K 31/554; A61K 31/426; A61K 31/43; A61K 31/5365; A61K 31/545

(52) U.S. Cl. 514/192; 514/200; 514/211.15; 514/230.5; 514/370

(57) ABSTRACT

The present invention relates to novel, low molecular weight broad spectrum compounds in particular to a class of 4-substituted or unsubstituted-7-hydro-1,4-thiazepine-7-[bicyclic or tricyclic heteroaryl] substituted-3,6-dicarboxylic acid or their derivatives which have β-lactamase inhibitory and antibacterial properties. The compounds are therefore useful in the treatment of antibacterial infections in humans or animals, either alone or in combination with other antibiotics.

This application is a Continuation of U.S. application Ser. No. 10/834,301 filed on Apr. 28, 2004, which claims the benefit under 35 U.S.C. § 119(e) of U.S. Provisional Application No. 60/465,906, filed Apr. 28, 2003, the entire disclosures of which are hereby incorporated by reference.

### FIELD OF INVENTION

This invention relates to certain 4-substituted or unsubstituted-7-hydro-1,4-thiazepine-7-[bicyclic or tricyclic heteroaryl] substituted-3,6-dicarboxylic acid derivatives which act as a broad spectrum β-lactamase inhibitors. β-Lactamases hydrolyze β-lactam antibiotics, and as such serve as the primary cause of bacterial resistance. The compounds of the present invention when combined with β-lactam antibiotics will provide an effective treatment against life threatening bacterial infections.

### BACKGROUND OF THE INVENTION

Penicillins and cephalosporins are the most frequently and widely used β-lactam antibiotics in the clinic. However, the development of resistance to β-lactam antibiotics by different pathogens has had a damaging effect on maintaining the effective treatment of bacterial infections. (Coleman, K. Expert Opin. Invest. Drugs 1995, 4, 693; Sutherland, R. Infection 1995, 23 (4) 191; Bush, K, Cur. Pharm. Design 1999, 5, 839-845) The most significant known mechanism related to the development of bacterial resistance to the β-lactam antibiotics is the production of class-A, class-B and class-C serine β-lactamases. These enzymes degrade the β-lactam antibiotics, resulting in the loss of antibacterial activity. Class-A enzymes preferentially hydrolyze penicillins where as Class-C lactamases have a substrate profile favoring cephalosporin hydrolysis. (Bush, K.; Jacoby, G. A.; Medeiros, A. A. Antimicrob. Agents Chemother. 1995, 39, 1211). To date over 250 different β-lactamases have been reported (Payne, D. J,: Du, W and Bateson, J. H. Exp. Opin. Invest Drugs 2000, 247.) and there is a need for a new generation of broad spectrum β-lactamase inhibitors. Bacterial resistance to these antibiotics could be greatly reduced by administering the β-lactam antibiotic in combination with a compound which inhibits these enzymes.

The commercially available β-lactamase inhibitors such as clavulanic acid, sulbactam and tazobactam are all effective against class-A producing pathogens. Clavulanic acid is clinically used in combination with amoxicillin and ticarcillin; similarly sulbactam with ampicillin and tazobactam with piperacillin. However, these compounds are ineffective against class C producing organisms. The mechanism of inactivation of class-A β-lactamases (such as PCI and TEM-1) has been elucidated. (Bush, K.; Antimicrob. Agents Chemother. 1993, 37, 851; Yang, Y.; Janota, K.; Tabei, K.; Huang, N.; Seigal, M. M.; Lin, Y. I.; Rasmussen, B. A. and Shlaes, D. M. J. Biol. Chem. 2000, 35, 26674-26682).

In 1981, the Beecham group disclosed 6-alkylidine penems of general structure 1 as inhibitors of β-lactamases. [N. F. Osborne, U.S. Pat. No. 4,485,110 (1984); N. F. Osborne, Eur. Pat. Appl. 81 301683.9, 1981; N. F. Osborne, Eur. Pat. Appl. 84301255.0; N. F. Osborne, Eur. Pat. Appl. 85100520.7; Eur. Pat. Appl. 85100521.5; Eur. Pat. Appl. 85300456-2; N. J. P. Broom; F. P. Harrington, PCT WO 94/10178; K. Coleman; J. E. Neale PCT WO 95/28935; K. Coleman; J. E. Neale PCT WO 95/17184]

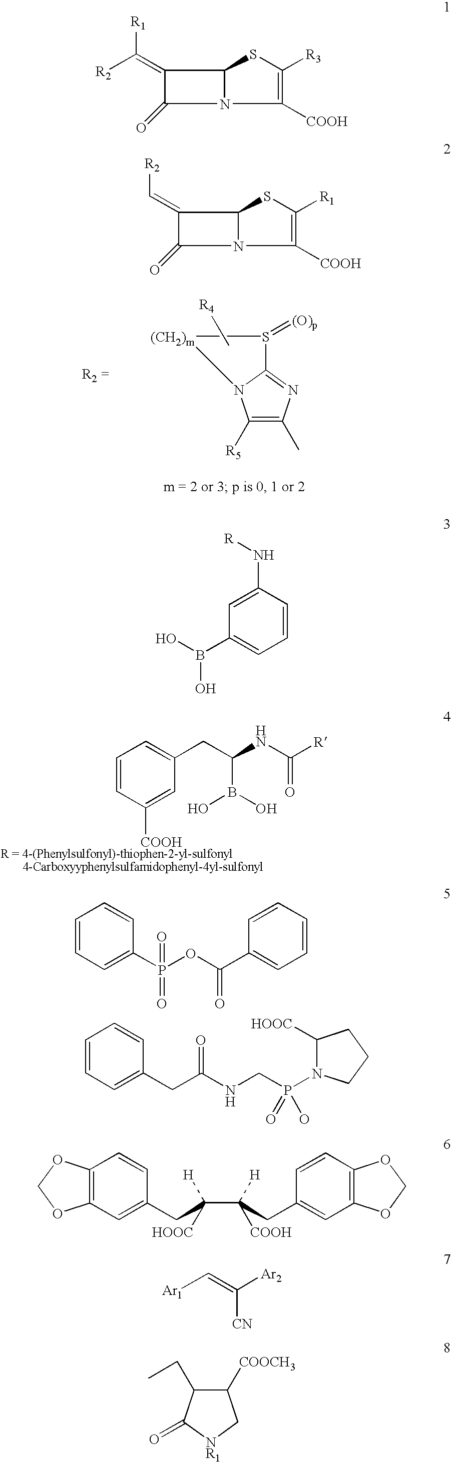

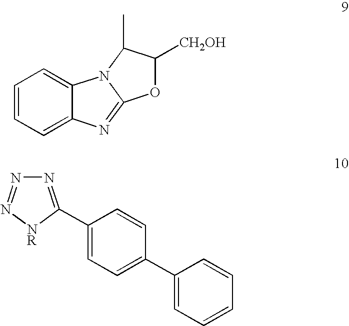

In addition to these methylidene based broad spectrum inhibitors, there were several transition state analogs based on boronic acids 3, 4 and acyl phosphanate 5 based inhibitors have been designed. [Martine, R.; Jones, J. B. Tetrahedron Lett. 1995, 36, 8399; Martine, R.; Gold, M.; Jones, J. B. Bioorg. Med. Chem. Lett. 1994, 4, 1229; Curley, K.; Pratt, R. F. J. Am. Chem. Soc. 1997, 119, 1529.; Rahil, J.; Pratt, R. F.; Biochem. J. 1991, 275, 793.; Rahil, J.; Pratt, R. F.; Biochemistry 1993, 32, 10763.]

The phosphonate based inhibitors were found to exclusively inhibit only the class C enzymes. In addition to these inhibitors several substituted succinic acid derivatives 6 were found to be potent inhibitors of metallo-β-lactamases. [Arakawa, Y.; Murakami, M.; Suzuki, K.; Ito, H.; Wacharotayankun, R.; Ohsuka, S.; Kato, N.; Ohta, M. Antimicrob. Agents Chemother., 1996, 40, 349.] A number of compounds such as acrylonitrile derivatives 7, oxopyrrolidine carboxylate derivatives 8, 2H, 3H-benzimidazo[2,1-b]oxazole derivatives 9 and biphenyl tetrazole are reported to have weak β-lactamase inhibitor activity. [For recent review ref. (a) Payne, D. J.; Du, W.; Bateson, J. H.; Exp. Opin. Invest. Drugs 2000, 9, 247: (b) Sandanayaka, V. P.; Prashad, A. S. Current. Med. Chem. 2002, 9, 1145: (c) Micetich, R. G.; salama, S. M.; Maiti, S. N.; Reddy, A. V. N.; Singh, R. Current. Med. Chem. 2002, 1, 193.] How ever, to-date there is no known β-lactamase inhibitor having the molecular structure of 4,7-dihydro-1,4-thiazepine-7-[bicyclic or tricyclic heteroaryl] substituted-3,6-dicarboxylic acid or their derivatives. However 1,4-thiazepine derivatives have been prepared in the past by three groups. [(a) Broom, N. J. P.; Farmer, T. H.; Osborne, N. F.; Tyler, J. W. J. Chem. Soc. Chem. Comm. 1992, 1663; (b) Visentin, G.; Perrone, E.; Borghi, D.; Rizzo, V.; Alpegiani, M.; Bedeschi, A.; Corigli, R.; Rivola, G.; Franceschi, G. Heterocycles, 1992, 33, 859; (c) Didier, B.; Pierre, D.; Dominique, L.; Eliane, M.; Antonio, U. PCT, WO 2000/005246.]

### SUMMARY OF THE INVENTION

The present invention relates to novel, low molecular weight broad spectrum compounds in particular to a class of 4-substituted or unsubstituted-7-hydro-1,4-thiazepine-7-[bicyclic or tricyclic heteroaryl] substituted-3,6-dicarboxylic acid or their derivatives which have β-lactamase inhibitory and antibacterial properties. The compounds are therefore useful in the treatment of antibacterial infections in humans or animals, either alone or in combination with other antibiotics.

In accordance with the present invention there are provided compounds of general formula I or a pharmaceutically acceptable salt or in vivo hydrolyzable ester

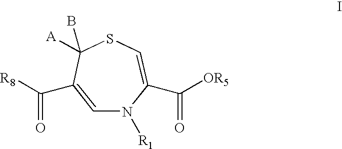

wherein:

One of A and B denotes hydrogen and the other an optionally substituted fused bicyclic or tricyclic heteroaryl group.

R₁ is H, optionally substituted —C1-C6 alkyl, optionally substituted -aryl, optionally substituted -heteroaryl or mono or bicyclic saturated heterocycles, optionally substituted —C3-C7 cycloalkyl, optionally substituted —C3-C6 alkenyl, optionally substituted —C3-C6 alkynyl with the proviso that both the double bond and the triple bond should not be present at the carbon atom which is directly linked to N; optionally substituted —C1-C6 per fluoro alkyl, —S(O)ₚ optionally substituted alkyl or aryl where p is 2, optionally substituted —C═Oheteroaryl, optionally substituted —C═Oaryl, optionally substituted —C═O(C1-C6) alkyl, optionally substituted —C═O(C3-C6) cycloalkyl, optionally substituted —C═O mono or bicyclic saturated heterocycles, optionally substituted C1-C6 alkyl aryl, optionally substituted C1-C6 alkyl heteroaryl, optionally substituted aryl-C1-C6 alkyl, optionally substituted heteroaryl-C1-C6 alkyl, optionally substituted C1-C6 alkyl mono or bicyclic saturated heterocycles, optionally substituted arylalkenyl of 8 to 16 carbon atoms, —CONR₆R₇, —SO₂NR₆R₇, optionally substituted arylalkyloxyalkyl, optionally substituted -alkyl-O-alkyl-aryl, optionally substituted -alkyl-O-alkyl-heteroaryl, optionally substituted aryloxyalkyl, optionally substituted heteroaryloxyalkyl, optionally substituted aryloxyaryl, optionally substituted aryloxyheteroaryl, optionally substituted C1-C6alkyl aryloxyaryl, optionally substituted C1-C6 alkyl aryloxyheteroaryl, optionally substituted alkyl aryloxy alkylamines, optionally substituted alkoxy carbonyl, optionally substituted aryloxy carbonyl, optionally substituted heteroaryloxy carbonyl. Preferred R₁ groups are H, optionally substituted alkyl, optionally substituted aryl, —C═O(C1-C6)alkyl, C3-C6alkenyl, C3-C6alkynyl, optionally substituted cycloalkyl, SO₂alkyl, SO₂aryl, optionally substituted heterocycles, —CONR₆R₇, and optionally substituted heteroaryl.

R₂ is hydrogen, optionally substituted C1-C6 alkyl, optionally substituted C2-C6 alkenyl having 1 to 2 double bonds, optionally substituted C2-C6 alkynyl having 1 to 2 triple bonds, halogen, cyano, N—R₆R₇, optionally substituted C1-C6 alkoxy, hydroxy; optionally substituted aryl, optionally substituted heteroaryl, COOR₆, optionally substituted alkyl aryloxy alkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylene dioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted C1-C6 alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, optionally substituted alkylaryloxyalkylamines, optionally substituted C3-C7 cycloalkyl, optionally substituted C3-C7 saturated or partially saturated heterocycle. Preferred R₂ groups are H, optionally substituted alkyl, optionally substituted alkoxy, optionally substituted heteroaryl, halogen, CN, hydroxy, optionally substituted heterocycle, —CONR₆R₇, COOR₆, optionally substituted aryl, S(O)_{q}-alkyl, and S(O)_{q}-aryl.

R₃ is hydrogen, C1-C6 alkyl, C3-C6 cycloalkyl, optionally substituted aryl, optionally substituted heteroaryl. Preferred R₃ groups are H or C1-C6 alkyl.

R₄ is H, optionally substituted C1-C6 alkyl, one of R₄ is OH, C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S═(O)n (where n=0 to 2), N—R₁; preferred R₄ groups are H, C1-C6 alkyl, NR₆R₇, or R₄R₄ together with the carbon to which they are attached forming a spiro system of five to eight members.

R₅ is H, an in vivo hydrolyzable ester such as C1-C6 alkyl, C5-C6 cycloalkyl, CHR₃OCOC1-C6 or salts such as Na, K, Ca; preferably R₅ is H or a salt;

R₆ and R₇ are independently H, optionally substituted C1-C6 alkyl, optionally substituted aryl, optionally substituted heteroaryl, optionally substituted C1-C6 alkyl aryl, optionally substituted arylalkyl, optionally substituted heteroarylalkyl, optionally substituted C1-C6 alkyl heteroaryl, R₆ and R₇ can be together to form a 3-7 membered saturated ring system optionally having one or two heteroatoms such as N—R₁, O, S═(O)ₙ n=0-2. Preferred R₆ and R₇ groups are H, C1-C6 alkyl, arylalkyl, heteroarylalkyl or R₆ and R₇ together forming a 3-7 membered saturated ring system optionally having one or two heteroatoms.

R₈ is N—R₆R₇, optionally substituted C1-C6 alkoxy, hydroxy, optionally substituted —S—C1-C6 alkyl, optionally substituted —O-Aryl, optionally substituted —O—C1-C6-alkyl-aryl, optionally substituted —O-aryl alkyl(C1-C6), optionally substituted —S-Aryl, optionally substituted —S—C1-C6-alkyl-aryl, optionally substituted —S-aryl-alkyl(C1-C6), optionally substituted —O-Aryl-C1-C6-NR₆R₇, optionally substituted —S-Aryl-C1-C6-NR₆R₇, —S— optionally substituted C1-C6 alkyl-COO—H, —S— optionally substituted C1-C6 alkyl-COO—C1-C6 alkyl.

### DETAILED DESCRIPTION OF THE INVENTION

In accordance with the present invention there are provided compounds of general formula I or a pharmaceutically acceptable salt or in vivo hydrolyzable ester R₅ thereof:

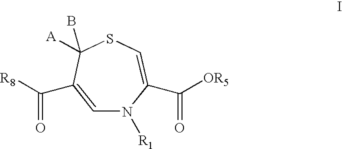

wherein:

One of A and B denotes hydrogen and the other an optionally substituted fused bicyclic heteroaryl group or an optionally substituted fused tricyclic heteroaryl group.

The expression “Fused tricyclic heteroaryl group” is used in the specification and claims to mean:

a group comprising three fused rings in which at least one ring has aromatic character (i.e meets Huckel's rule (4n+2)). The fused tricyclic heteroaryl group contains 1-6 heteroatoms selected from the group consisting of O, S, N and N—R₁. The fused tricyclic heteroaryl must be bonded through a carbon preferably in one of the at least one aromatic rings to the remainder of the formula I molecule. The fused tricyclic heteroaryl group may contain 1-3 aromatic rings and 0-2 non-aromatic rings. Each aromatic ring(s) in the fused tricyclic heteroaryl group may contain 5 to 7 ring atoms (including the bridgehead atoms) selected from CR₂, O, S, N, and N—R₁. Each of the aromatic ring(s) of the fused tricyclic heteroaryl group may contain 0 to 3 heteroatoms selected from O, S, N or N—R₁. The non-aromatic ring(s), if any, of the fused tricyclic heteroaryl group may contain 5-8 ring atoms (including bridgehead atoms) and contain 0-4 heteroatoms selected from N, N—R₁, O or S(O)ₙ, wherein n is 0-2. In each non-aromatic ring of the fused tricyclic heteroaryl group, one or two of the non-bridgehead carbon atoms may each be optionally substituted with one or two R₄, and each R₄ may be independently the same or different. Examples of fused tricyclic heteroaryl are optionally substituted ring systems such as imidazo[2,1-b][1,3]benzothiazole optionally substituted e.g., by for example C1-C6alkyl, C1-C6alkoxy or halo (such as chlorine or fluorine); imidazo[1,2-a]quinoline; 6,7-dihydro-5H-cyclopenta[d]imidazo[2,1-b][1,3]thiazole; imidazo[1,2-a]quinoxaline; 5,6,7,8-tetrahydro-[1,2,4]triazolo[1,5-a]pyridine dibenzo[b,f][1,4]-oxazepin-11(10H)-one optionally substituted e.g., by arylalkyl such as benzyl; 7,8-dihydro-6H-3,4,8b-triaza-as-indacene optionally substituted by C1-C6 alkoxy; 4H, 10H-pyrazolo[5,1-c][1,4]benzoxazepine optionally substituted e.g., by C1-C6 alkoxy; 5H-Imidazo[2,1-a]isoindole; 5,8-dihydro-6H-imidazo[2,1-b]pyrano[4,3-d][1,3]thiazole; imidazo[2,1-b]benzothiazole; [1,3]thiazolo[3,2-a]benzimidazole; 7,8-dihydro-6H-cyclopenta[3,4]pyrazolo[5,1-b][1,3]thiazole; 5,6,7,8-tetrahydroimidazo[2,1-b][1,3]-benzothiazole; 9H-imidazo[1,2-a]benzimidazole optionally substituted e.g., by C1-C6alkyl; 4H-thieno[2′,3′:4,5]thiopyrano[2,3-b]pyridine; 7,8-dihydro-6H-cyclopenta[e][1,2,4]-triazolo[1,5-a]pyrimidine optionally substituted e.g., by C1-C6alkyl; 6,7,8,9-tetrahydropyrido[3,4-e][1,2,4]triazolo[1,5-a]pyrimidine optionally substituted e.g., by C2-C7alkoxycarbonyl; 8′,9′-dihydro-6′H-spiro[1,3-dioxolane-2,7′-[1,2,4]triazolo[1,5-a]-quinazoline; 6,7,8,9-tetrahydro[1,2,4]triazolo[1,5-a]quinazoline optionally substituted e.g., by C1-C6alkyl; 7,8-dihydro-6H-cyclopenta[e]imidazo[1,2-a]pyrimidine optionally substituted e.g., by C1-C6alkoxy; 7,8-dihydro-6H-cyclopenta[e]imidazo[1,2-a]pyrimidinyl optionally substituted e.g., by arylalkyloxyalkyloxy; 3-dihydro[1,3]thiazolo[3,2-a]-benzimidazole; 2,3-dihydro[1,3]thiazolo[3,2-a]benzimidazole; 4-dihydro-2H-[1,3]thiazino[3,2-a]-benzimidazole; [1,3]thiazolo[3,2-a]benzimidazole; 7,8-dihydro-5H-pyrano[4,3-d]pyrazolo[5,1-b][1,3]-oxazole; 5,6,7,8-tetrahydropyrazolo[5,1-b][1,3]benzoxazole; and 5,6,7,8-tetrahydropyrazolo[5′, 1′:2,3][1,3]oxazolo[5,4-c]pyridine optionally substituted e.g., by C2-C7alkoxycarbonyl.

Further examples of fused tricyclic heteroarylgroups are as follows:

Examples of fused tricyclic heteroarylgroup A and B:

Ring size and arrangements: (5-5-5)

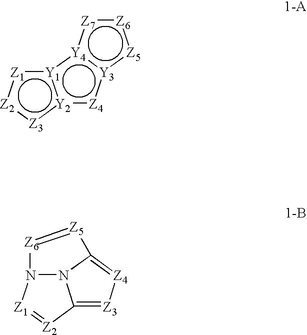

In both formula 1-A and 1-B Z₁, Z₂, Z₃, Z₄, Z₅, Z₆ and Z₇ are independently selected from CR₂, N, O, S or N—R₁, and as mentioned above one of Z₁-Z₇ is a carbon atom to which the remainder of the molecule is attached. Y₁, Y₂, Y₃ and Y₄ may independently be C or N.

Ring size and arrangement: (5-5-6)

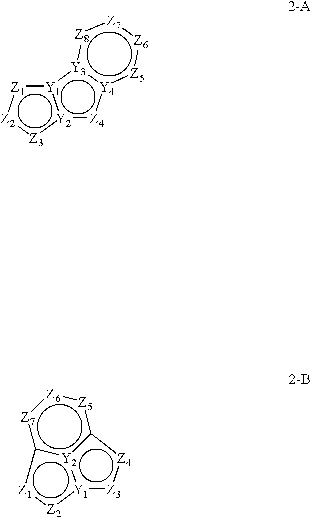

In both formula 2-A and 2-B Z₁, Z₂, Z₃, Z₄, Z₅, Z₆, Z₇ and Z₈ are independently selected from CR₂, N, O, S or N—R₁ and as mentioned above one of the Z₁-Z₈ is a carbon atom to which the remainder of the molecule is attached. Y₁, Y₂, Y₃ and Y₄ may be independently be C or N.

Ring size and arrangement: (5-6-5)

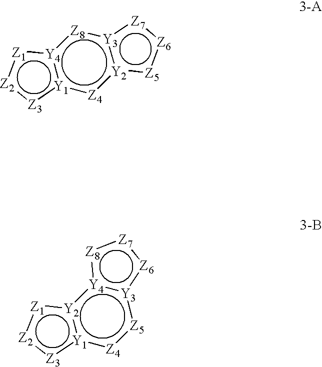

In both formula 3-A and 3-B Z₁, Z₂, Z₃, Z₄, Z₅, Z₆, Z₇ and Z₈ are independently selected from CR₂, N, O, S or N—R₁ and as mentioned above one of Z₁-Z₈ is a carbon atom to which the remainder of the molecule is attached. Y₁, Y₂, Y₃ and Y₄ may be C or N.

Ring size and arrangements: (5-6-6)

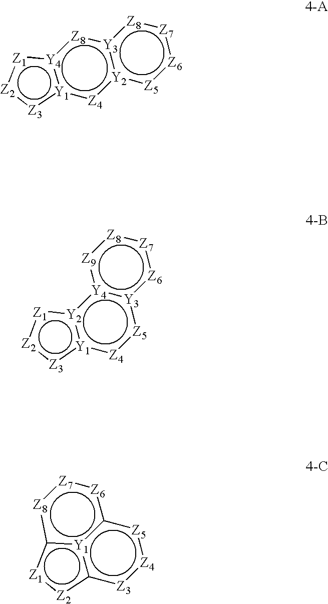

In formula 4-A, 4-B and 4-C Z₁, Z₂, Z₃, Z₄, Z₅, Z₆, Z₇ and Z₈ are independently selected from CR₂, N, O, S or N—R₁ and as mentioned above one of the Z₁-Z₈ is a carbon atom to which the remainder of the molecule is attached. Y₁, Y₂, Y₃ and Y₄ are independently C or N.

Ring size and arrangements: [5-5-(non-aromatic)]

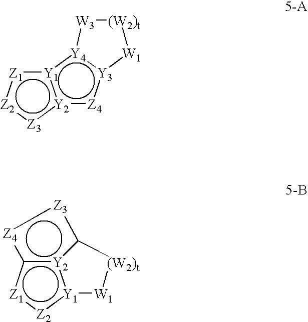

In both formula 5-A and 5-B Z₁, Z₂, Z₃ and Z₄ are independently selected from CR₂, N, O, S or N—R₁ and as mentioned above one of the Z₁-Z₄ is a carbon atom to which the remainder of the molecule is attached; Y₁, Y₂, Y₃ and Y₄ are independently C or N. W₁, W₂ and W₃ are independently selected from CR₄R₄, S(O)r (r=0-2), O, N—R₁ with the proviso that no S—S, S—O or O—O bond formation can occur to form a saturated ring; and t=1 to 3.

Ring size and arrangement: [5-6-(non-aromatic)]

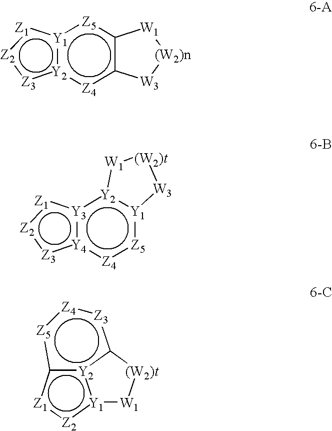

In formulae 6-A, 6-B and 6-C Z₁, Z₂, Z₃, Z₄ and Z₅ are independently selected from CR₂, N, O, S or N—R₁ and as mentioned above one of the Z₁-Z₅ is a carbon atom to which the remainder of the molecule is attached. Y₁, and Y₂ are independently C or N. W₁, W₂ and W₃ are independently CR₄R₄, S(O)r (r=0-2), O, or N—R₁ with the proviso that no S—S, S—O or O—O bond formation can occur to form a saturated ring; and t=1 to 3.

Ring size and arrangement: [5-(non-aromatic)-5]

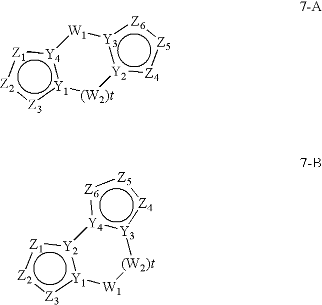

In formulae 7-A and 7-B Z₁, Z₂, Z₃, Z₄, Z₅ and Z₆ are independently selected from CR₂, N, O, S, and N—R₁; one of Z₁-Z₆ is a carbon atom to which the remainder of the molecule is attached. Y₁, Y₂, Y₃ and Y₄ are independently C or N. W₁ and W₂ are independently selected from CR₄R₄, S(O)r (r=0-2), O, N—R₁ with the proviso that no S—S, S—O or O—O bond formation can occur to form a saturated ring; and t=1 to 3.

Ring size and arrangement: [5-(non-aromatic)-6]

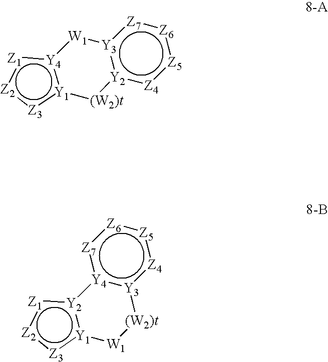

In formulae 8-A and 8-B Z₁, Z₂, Z₃, Z₄, Z₅, Z₆ and Z₇ are independently selected from CR₂, N, O, S and N—R₁ and as mentioned above one of the Z₁-Z₇ is a carbon atom to which the remainder of the molecule is attached. Y₁, Y₂, Y₃ and Y₄ are independently C or N. W₁ and W₂ are independently CR₄R₄, S(O)r (r=0-2), O, or N—R₁ with the proviso that no S—S, S—O or O—O bond formation can occur to form a saturated ring; and t=0-3.

Ring size and arrangement [5-(non-aromatic)-(non-aromatic)]

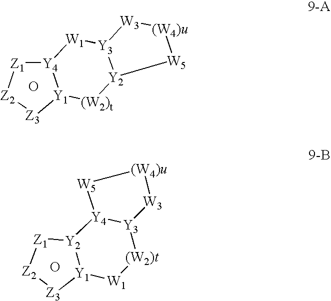

In formulae 9-A and 9-B Z₁, Z₂ and Z₃ are independently selected from CR₂ N, O, S or N—R₁; one of Z₁-Z₃ is a carbon atom to which the remainder of the molecule is attached. Y₁ and Y₄ are independently C or N; Y₂ and Y₃ are independently CH or N; W₁, W₂ W₃, W₄ and W₅ are independently CR₄R₄, S(O)r (r=0-2), O, or N—R₁ with the proviso that no S—S, S—O or O—O bond formation can occur to form a saturated ring; t=0 to 2 and u=1 to 3.

Ring size and arrangement (6-5-6)

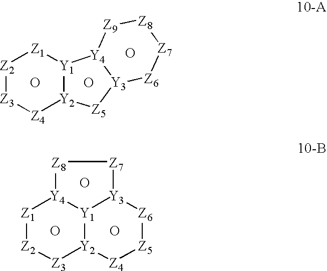

In formula 10-A and 10-B Z₁, Z₂, Z₃, Z₄, Z₅, Z₆, Z₇, Z₈ and Z₉ are independently selected from CR₂, N, O, S or N—R₁ and as mentioned above one of the Z₁-Z₉ is a carbon atom to which the remainder of the molecule is attached. Y₁, Y₂, Y₃ and Y₄ are independently C or N.

Ring size and arrangement (6-6-6)

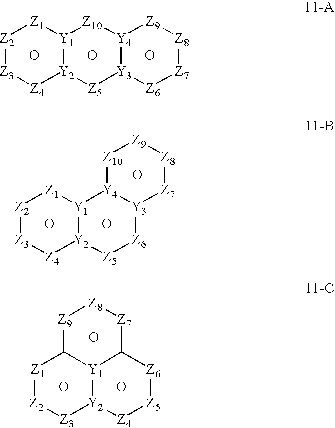

In formula 11-A, 11-B and 11-C Z₁, Z₂, Z₃, Z₄, Z₅, Z₆, Z₇, Z₈, Z₉ and Z₁₀ are independently CR₂, N, O, S or N—R₁; one of the Z₁-Z₁₀ is a carbon atom to which the remainder of the molecule is attached. Y₁, Y₂, Y₃ and Y₄ are independently C or N.

Ring size and arrangement [6-5-(non-aromatic)]

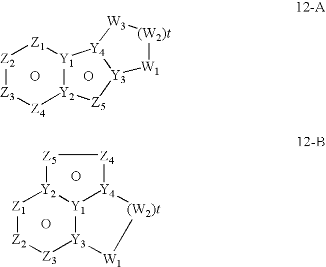

In formula 12-A and 12-B Z₁, Z₂, Z₃, Z₄ and Z₅ are independently CR₂, N, O, S or N—R₁ with the proviso that one of Z₁-Z₅ is a carbon atom to which the remainder of the molecule is attached. Y₁, Y₂, Y₃ and Y₄ are independently C or N; W₁, W₂, W₃ are independently CR₄R₄ O, N—R₁, or S═(O)ᵣ (r=0-2) with the proviso that no S—S, S—O or O—O bond formation can occur to form a saturated ring; and t=1-4.

Ring size and arrangement [6-6-(non-aromatic)]

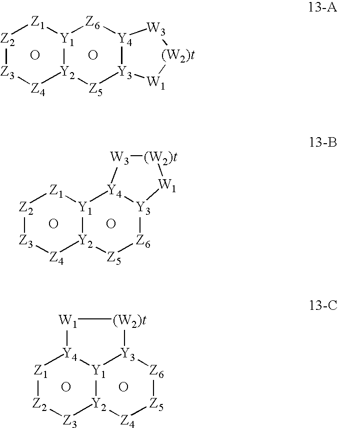

In formula 13-A, 13-B and 13-C Z₁, Z₂, Z₃, Z₄, Z₅ and Z₆ are independently CR₂, N, O, S or N—R₁; one of Z₁-Z₆ is a carbon atom to which the remainder of the molecule is attached. Y₁, Y₂, Y₃ and Y₄ are independently C or N; W₁, W₂ and W₃ are independently CR₄R₄, S(O)r (r=0-2), O, or N—R₁ with the proviso that no S—S, S—O or O—O bond formation can occur to form a saturated ring; and t=1 to 3.

Ring size and arrangement [6-(non-aromatic)-6]

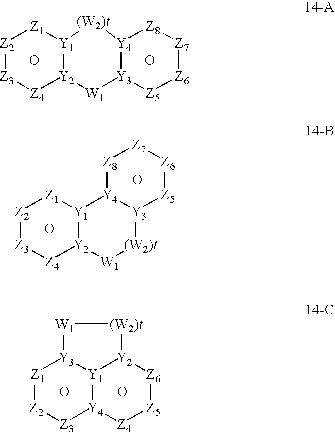

In formula 14-A, 14-B and 14-C Z₁, Z₂, Z₃, Z₄, Z₅, Z₆, Z₇ and Z₈ are independently CR₂, N, O, S or N—R₁; one of Z₁-Z₈ is a carbon atom to which the remainder of the molecule is attached. Y₁, Y₂, Y₃ and Y₄ are independently C or N; W₁, and W₂ are independently CR₄R₄, S(O)r (r=0-2), O, or N—R₁ with the proviso that no S—S, S—O or O—O bond formation can occur to form a saturated ring; and t=1 to 2.

Ring size and arrangement [6-(non-aromatic)-(non-aromatic)]

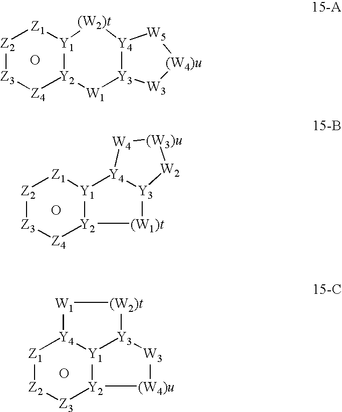

In formula 15-A, 15-B and 15-C Z₁, Z₂, Z₃ and Z₄ are independently CR₂, N, O, S or N—R₁; one of Z₁-Z₄ is a carbon atom to which the remainder of the molecule is attached. Y₁, Y₂, Y₃ and Y₄ are independently C or N; W₁, W₂, W₃, W₄ and W₅ are independently CR₄R₄, S(O)r (r=0-2), O, or N—R₁ with the proviso that no S—S, S—O or O—O bond formation can occur to form a saturated ring; t=1 to 3 and u=1 to 3.

The preferred embodiments of formula 1-A are:
1. Z₁ is O, S, or N—R₁; Z₂, Z₃, Z₄, Z₅, Z₆ and Z₇ are independently CR₂, or N; Y₁, Y₂, Y₃, Y₄ are C; any one of Z₂, Z₃, Z₄, Z₅, Z₆, Z₇ is a carbon to which the remainder of the molecule is attached.
2. Z₁ is O, S, or N—R₁; Z₂, Z₃, Z₄, Z₅, Z₆ and Z₇ are independently CR₂; Y₁, Y₂, Y₃, and Y₄ are C; one of Z₂, Z₃, Z₄, Z₅, Z₆, Z₇ is a carbon to which the remainder of the molecule is attached.
3. Z₂ is O, S, or N—R₁; Z₁, Z₃, Z₄, Z₅, Z₆ and Z₇ are independently CR₂, or N; Y₁, Y₂, Y₃, Y₄ are C; one of Z₁, Z₃, Z₄, Z₅, Z₆, Z₇ is a carbon to which the remainder of the molecule is attached.
4. Z₂ is O, S, or N—R₁; Z₁, Z₃, Z₄, Z₅, Z₆ and Z₇ are independently CR₂; Y₁, Y₂, Y₃, Y₄ are C; one of Z₁, Z₃, Z₄, Z₅, Z₆, Z₇ is a carbon to which the remainder of the molecule is attached.
5. Z₃ is O, S, or N—R₁; Z₁, Z₂, Z₄, Z₅, Z₆, Z₇ are independently CR₂, or N; Y₁, Y₂, Y₃, Y₄ are C; one of Z₁, Z₂, Z₄, Z₅, Z₆, Z₇ is a carbon to which the remainder of the molecule is attached.
6. Z₃ is O, S, or N—R₁; Z₁, Z₃, Z₄, Z₅, Z₆, Z₇ are independently CR₂; Y₁, Y₂, Y₃, Y₄ are C; one of Z₁, Z₃, Z₄, Z₅, Z₆, or Z₇ is a carbon to which the remainder of the molecule is attached.
7. Z₇ is O, S, or N—R₁; Z₁, Z₂, Z₃, Z₄, Z₅ and Z₆ are independently CR₂ or N; Y₁, Y₂, Y₃, Y₄ are C; one of Z₁, Z₂, Z₃, Z₄, Z₅ and Z₆ is a carbon to which the remainder of the molecule is attached.
8. Z₇ is O, S, or N—R₁; Z₁, Z₂, Z₃, Z₄, Z₅ and Z₆ are independently CR₂; Y₁, Y₂, Y₃, Y₄ are C; any of Z₁, Z₂, Z₃, Z₄, Z₅ and Z₆ is a carbon to which the remainder of the molecule is attached.
9. Z₁, Z₄, and Z₆ are independently O, S, or N—R₁; Z₂, Z₃, Z₅, Z₇ are independently CR₂, or N; Y₁, Y₂, Y₃, Y₄ are C; any one of Z₂, Z₃, Z₅, or Z₇ is a carbon to which the remainder of the molecule is attached.
10. Z₁, Z₄, and Z₆ are independently O, S, or N—R₁; Z₂, Z₃, Z₅, Z₇ are independently CR₂; Y₁, Y₂, Y₃, Y₄ are C; any one of Z₂, Z₃, Z₅, or Z₇ is a carbon to which the remainder of the molecule is attached.
11. Z₃, Z₄, and Z₆ are independently O, S, or N—R₁; Z₁, Z₂, Z₅, Z₇ are independently CR₂, or N; Y₁, Y₂, Y₃, Y₄ are C; any one of Z₁, Z₂, Z₅, or Z₇ is a carbon to which the remainder of the molecule is attached.
12. Z₃, Z₄, and Z₆ are independently O, S, or N—R₁; Z₂, Z₃, Z₅, Z₇ are independently CR₂; Y₁, Y₂, Y₃, Y₄ are C; any one of Z₁, Z₂, Z₅, or Z₇ is a carbon to which the remainder of the molecule is attached.
13. Z₁ is O, S, or N—R₁; Z₂, Z₃, Z₄, Z₅, Z₆ and Z₇ are independently CR₂, or N; Y₂ is N; Y₁, Y₃, Y₄ are C; any one of Z₂, Z₃, Z₄, Z₅, Z₆, or Z₇ is a carbon to which the remainder of the molecule is attached.
14. Z₁ is O, S, or N—R₁; Z₂, Z₃, Z₄, Z₅, Z₆ and Z₇ are independently CR₂; Y₂ is N; Y₁, Y₃, Y₄ are C; one of Z₂, Z₃, Z₄, Z₅, Z₆, or Z₇ is a carbon to which the remainder of the molecule is attached.
15. Z₂ and Z₄ are independently O, S, or N—R₁; Z₁, Z₃, Z₅, Z₆, Z₇ are independently CR₂, N; Y₁ is N; Y₂, Y₃, Y₄ are C; any one of Z₁, Z₃, Z₅, Z₆, or Z₇ is a carbon to which the remainder of the molecule is attached.
16. Z₂ and Z₄ are independently O, S, or N—R₁; Z₁, Z₃, Z₅, Z₆, Z₇ are independently CR₂; Y₁ is N; Y₂, Y₃, Y₄ are C; any one of Z₁, Z₃, Z₅, Z₆, or Z₇ is a carbon to which the remainder of the molecule is attached
17. Z₃ and Z₅ are independently O, S, or N—R₁; Z₁, Z₂, Z₄, Z₆, Z₇ are independently CR₂, or N; Y₁ is N; Y₂, Y₃, Y₄ are C; any one of Z₁, Z₃, Z₅, Z₆, or Z₇ is a carbon to which the remainder of the molecule is attached.
18. Z₃ and Z₅ are independently O, S, or N—R₁; Z₁, Z₂, Z₅, Z₆, Z₇ are independently CR₂; Y₁ is N; Y₂, Y₃, Y₄ are C; any one of Z₁, Z₃, Z₅, Z₆, or Z₇ is a carbon to which the remainder of the molecule is attached.
19. Z₁ and Z₅ are independently O, S, or N—R₁; Z₂, Z₃, Z₄, Z₆, Z₇ are independently N, or CR₂; Y₁ is N; Y₂, Y₃, Y₄ are C; any one of Z₂, Z₃, Z₄, Z₆, Z₇ is a carbon to which the remainder of the molecule is attached.
20. Z₁ and Z₅ are independently O, S, or N—R₁; Z₂, Z₃, Z₄, Z₆, Z₇ are independently CR₂; Y₁ is N; Y₂, Y₃, Y₄ are C; any one of Z₂, Z₃, Z₄, Z₆, or Z₇ is a carbon to which the remainder of the molecule is attached.
21. Z₃ and Z₇ are independently O, S, or N—R₁; Z₁, Z₂, Z₄, Z₅, Z₆ are independently N, or CR₂; Y₁ is N; Y₂, Y₃, Y₄ are C; any one of Z₁, Z₂, Z₄, Z₅, or Z₆ is a carbon to which the remainder of the molecule is attached.
22. Z₃ and Z₇ are independently O, S, N—R₁; Z₁, Z₂, Z₄, Z₅, Z₆ are independently CR₂; Y₁ is N; Y₂, Y₃, Y₄ are C; Any one of Z₁, Z₂, Z₃, Z₄, Z₅, or Z₆ is a carbon to which the remainder of the molecule is attached.
23. Z₃ and Z₇ are independently O, S, N—R₁; Z₁, Z₂, Z₄, Z₅, Z₆ are independently N, or CR₂; Y₂ is N; Y₁, Y₃, Y₄ are C; any one of Z₁, Z₂, Z₄, Z₅, or Z₆ is a carbon to which the remainder of the molecule is attached.
24. Z₃ and Z₇ are independently O, S, or N—R₁; Z₁, Z₂, Z₄, Z₅, Z₆ are independently CR₂; Y₂ is N; Y₁, Y₃, Y₄ are C; any one of Z₁, Z₂, Z₄, Z₅, or Z₆ is a carbon to which the remainder of the molecule is attached.
25. Z₃ and Z₅ are independently O, S, N—R₁; Z₁, Z₂, Z₄, Z₆, Z₇ are independently N, or CR₂; Y₂ is N; Y₁, Y₃, Y₄ are C; any one of Z₁, Z₂, Z₄, Z₆, or Z₇ is a carbon to which the remainder of the molecule is attached.
26. Z₃ and Z₅ are independently O, S, or N—R₁; Z₁, Z₂, Z₄, Z₆, Z₇ are independently CR₂; Y₂ is N; Y₁, Y₃, Y₄ are C; any one of Z₁, Z₂, Z₄, Z₆, or Z₇ is a carbon to which the remainder of the molecule is attached.
   The preferred embodiment of formula 1-B is:
27. Z₁, Z₂, Z₃, Z₄, Z₅, Z₆ and Z₇ are independently CR₂.

The preferred embodiments of formula 2-A are:
28. Z₁ is CR₂; Z₂ is the carbon to which the remainder of the molecule is attached; Z₃ is N or CR₂; Z₄ is O, S, CR₂ or N—R₁; Z₅, Z₆, Z₇, Z₈ are independently CR₂ or N; Y₁ is N; Y₂, Y₃ and Y₄ are C.
29. Z₂ is CR₂; Z₁ is carbon to which the remainder of the molecule is attached; Z₃ is N or CR₂; Z₄ is O, S, CR₂ or N—R₁; Z₅, Z₆, Z₇, Z₈ are independently CR₂ or N; Y₁ is N; Y₂, Y₃ and Y₄ are C.
30. Z₁ is N, Z₂ is carbon to which the remainder of the molecule is attached; Z₃ is N or CR₂; Z₄ O, S, CR₂ or N—R₁; Z₅, Z₆, Z₇, Z₈ are independently CR₂ or N; Y₁ is N; Y₂, Y₃ and Y₄ are C.
31. Z₁, Z₂, Z₄ are independently CR₂ or N; Z₄ is O, S, CR₂ or N—R₁; Z₅, Z₆, Z₇, Z₈ are independently CR₂ or N and one of Z₅, Z₆, Z₇, or Z₈ is a carbon to which the remainder of the molecule is attached; Y₁ is N; Y₂, Y₃ and Y₄ is C.
32. Z₁ is CR₂ or N; Z₂ is CR₂: Z₃ is O, S or N—R₁; Z₄ is N or CR₂; Z₅, Z₆, Z₇, Z₈ are independently CR₂; Y₁ is N, or C; Y₂, Y₃ and Y₄ are C.
33. Z₁, Z₂, Z₃, Z₄, Z₅, Z₆, Z₇, Z₈ are independently N or CR₂; Y₁, Y₂, Y₃, Y₄ are C.
34. Z₁, Z₂, Z₅, Z₆, Z₇, Z₈ are independently N or CR₂; Z₃ and Z₄ are independently O, S, or N—R₁; Y₁, Y₂, Y₃, and Y₄ are C.
35. Z₁, Z₂, and Z₃ are independently CR₂ or N; Z₄ is O, S, CR₂ or N—R₁; Z₅, Z₆, Z₇, and Z₈ are independently CR₂ or N; Y₁ is N; Y₂, Y₃ and Y₄ are C.
36. Z₁ is N; Z₂ is CR₂; Z₃ is the carbon atom to which remainder of the molecule is attached; Z₄ is N; Z₅, Z₆, Z₇, Z₈ are independently N or CR₂; Y₁, Y₂, Y₃, Y₄ are independently N or C.

The preferred embodiment of formula 2-B is:
37. Z₁ and Z₄ are independently CR₂ or N; Z₂ and Z₃ are CR₂; Z₅, Z₆, Z₇ are independently CR₂ or N; Y₁ is C and Y₂ is N.
38. Z₁ is O, S, or N—R₁; Z₂ is CR₂; Z₃ is CR₂, or N; 4 is O, S, N—R₁, or CR₂: Z₅, Z₆, Z₇, Z₈ are independently N or CR₂; Y₁, Y₂, Y₃, and Y₄ are C; one of Z₂, Z₃, Z₅, Z₆, Z₇, or Z₈ is a carbon atom to which the remainder of the molecule is attached.

The preferred embodiments of formula 3-A are:
39. Z₁ is O, S, or N—R₁; Z₂ is N, or CR₂; Z₃ is CR₂; Z₅, Z₆, and Z₇ are independently N or CR₂; Z₄ and Z₈ are independently O, S, or N—R₁; Y₁, Y₂, Y₃, and Y₄ are C and one of Z₂, Z₅, Z₆, or Z₇ is a carbon atom to which the remainder of the molecule is attached.
40. Z₃ is O, S, or N—R₁; Z₂ is N, CR₂; Z₁ is CR₂; Z₅, Z₆, and Z₇ are independently N or CR₂; Z₄ and Z₈ are independently O, S, or N—R₁; Y₁, Y₂, Y₃, and Y₄ are C and one of Z₂, Z₅, Z₆, or Z₇ is a carbon atom to which the remainder of the molecule is attached.

The preferred embodiments of formula 3-B are:
41. Z₁ is O, S, or N—R₁; Z₂ is N or CR₂; Z₃ is CR₂; Z₄, Z₅, Z₆, Z₇, and Z₈ are independently N or CR₂; Y₁, Y₂, Y₃, and Y₄ are C; and one of Z₂, Z₅, Z₆, Z₇ is a carbon atom to which the remainder of the molecule is attached.
42. Z₁ is N or CR₂; Z₂ is CR₂; Z₃ is O, S, N—R₁ or CR₂; Z₇ is CR₂ or N; Z₆, and Z₈ are independently N or CR₂; Z₄ and Z₅ are CR₂ or N; Y₁, Y₂, and Y₃ are C; Y₄ is N and one of Z₂, Z₄, Z₅, Z₆ is a carbon atom to which the remainder of the molecule is attached.
43. Z₁ is N, or CR₂; Z₂ is CR₂; Z₃ is O, S, N—R₁ or CR₂; Z₆ is CR₂ or N; Z₇, and Z₈ are independently N or CR₂; Z₄ and Z₅ are independently CR₂ or N; Y₁, Y₂, Y₃ are C; Y₄ is N and one of Z₂, Z₄, Z₅, Z₆ is a carbon atom to which the remainder of the molecule is attached.
44. Z₁ is O, S, or N—R₁; Z₂ is N, or CR₂; Z₃ is CR₂; Z₆, Z₇, and Z₈ are N; 4 and Z₅ are independently CR₂ or N; Y₁, Y₂, Y₄ are C; Y₃ is N and one of Z₂, or Z₃ is a carbon atom to which the remainder of the molecule is attached.
45. Z₁ is N or CR₂; Z₂ is CR₂; Z₃ is O, S, N—R₁ or CR₂; Z₆, Z₇, and Z₈ are N; 4, and Z₅ are independently CR₂ or N; Y₁, Y₂, Y₄ are C; Y₃ is N and one of Z₁, Z₂ is a carbon atom to which the remainder of the molecule is attached.
46. Z₁ is N or CR₂; Z₂ is CR₂; Z₃ is O, S, N—R₁ or CR₂; Z₆, Z₇, and Z₈ are N; Z₄ and Z₅ are independently CR₂ or N; Y₁, Y₂, Y₄ are C; Y₃ is N and one of Z₁, Z₂, is a carbon atom to which the remainder of the molecule is attached.
47. Z₁ is N; Z₂, Z₃, Z₄ and Z₅ are independently CR₂; Z₆, Z₇, and Z₈ are independently O, S, N, N—R₁ or CR₂; Y₂, Y₃, and Y₄ are C; Y₁ is N; one of Z₂, Z₃, Z₆, Z₆, Z₇, Z₈ is a carbon atom to which the remainder of the molecule is attached.
48. Z₃ is N; Z₂ and Z₁ are independently CR₂; Z₄, and Z₅ are independently CR₂; Z₆, Z₇, Z₈ are independently O, S, N, N—R₁ or CR₂; Y₂, Y₃, and Y₁ are C; Y₄ is N; one of Z₂, Z₁, Z₆, Z₆, Z₇, Z₄ is a carbon atom to which the remainder of the molecule is attached.
49. Z₁ is N, or CR₂; Z₂ is CR₂; Z₃ is O, S, or N—R₁; Z₄ and Z₅ are independently CR₂; Z₆, Z₇, Z₈ are independently O, S, N, N—R₁, or CR₂; Y₁, Y₂, Y₃, and Y₄ are C; one of Z₁, Z₂, Z₃, Z₆, Z₇, Z₈ is a carbon atom to which the remainder of the molecule is attached.

The Preferred embodiments of formula 4-A
50. Z₁ and Z₃ are independently O, S, N—R₁, N, or CR₂; Z₂ is CR₂; Z₄, Z₅, Z₆, Z₇, Z₈, Z₉, are independently CR₂; Y₁, Y₂, Y₃, Y₄, are C.
51. Z₁ and Z₃ are independently O, S, N—R₁, N, or CR₂; Z₂ is CR₂; Z₈, and Z₉ are independently CR₂, or N; Z₅, Z₆, Z₇, Z₈ are independently CR₂; Y₁, Y₂, Y₃, Y₄, are C; One of Z₁, Z₂, Z₃, Z₅, Z₆, Z₇, Z₈ is a carbon atom to which the remainder of the molecule is attached.
52. Z₁ is S, O, or N—R₁; Z₂, Z₃, Z₄, Z₅, Z₆, Z₇, Z₈ are independently CR₂; Z₉ is N; Y₁, Y₂, Y₃, Y₄, are C.
53. Z₁, and Z₃ are independently O, S, N—R₁, N, or CR₂; Z₄, and Z₉, are independently N or CR₂; Z₅, Z₆, Z₇, Z₈ are independently CR₂ or N; Y₁, Y₂, Y₃, Y₄, are C; Z₂ is the carbon to which the remainder of the molecule is attached.
54. Z₁ is N; Z₂, Z₃, and Z₄ are independently CR₂; Z₅, Z₆, Z₇, Z₈ are independently N or CR₂; Z₉ is CR₂ or N; Y₁ is N; Y₂, Y₃, and Y₄, are C; Z₂ or Z₃ is the carbon to which the remainder of the molecule is attached.
55. Z₃ is N; Z₁, Z₂, and Z₄ are independently CR₂; Z₅, Z₆, Z₇, and Z₈ are independently CR₂, or N; Z₉ is CR₂, or N; Y₄ is N; Y₁, Y₂, and Y₃ are C.

The Preferred embodiments of formula 4-B
56. Z₁ is N; Z₂, Z₃, Z₄, Z₅, Z₆, Z₇, Z₈, and Z₉ are independently CR₂; Y₁ is N; Y₂, Y₃, and Y₄ are C; one of Z₂, Z₃, Z₆, Z₇, Z₈, Z₉ is the carbon atom to which the remainder of the molecule is attached.
57. Z₃ is N; Z₁, Z₂, and Z₄ are independently CR₂; Z₅, Z₆, Z₇, Z₈, and Z₉ are independently CR₂ or N; Y₁, Y₃, Y₄ are C; Y₂ is N.
58. Z₁ is O, S, or N—R₁; Z₂, Z₃, and Z₄ are independently CR₂; Z₅ is CR₂ or N; Z₆, Z₇, Z₈, and Z₉ are independently CR₂, or N; one of Z₆, Z₇, Z₈, Z₉ is a carbon atom to which the remainder of the molecule is attached.
59. Z₁ and Z₃ are independently O, S, N—R₁, N, or CR₂; Z₄ is CR₂ or N; Z₅ is CR₂; Z₆, Z₇, Z₈, and Z₉ are independently CR₂, or N; Y₁, and Y₂ are independently C or N; Y₃ and Y₄ are C.

The Preferred embodiments of formula 4-C
60. Z₁ and Z₂ are independently N or CR₂; Z₃, Z₄, Z₅, Z₆, Z₇, and Z₈ are independently CR₂; Y₁ is C.
61. Z₁, and Z₂ are independently CR₂; Z₃, Z₄, Z₅, Z₆, Z₇, and Z₈ are independently N or CR₂; Y₁ is C.
62. Z₁, Z₂, Z₃, Z₄, Z₅, Z₆, Z₇, and Z₈ are independently N or CR₂; Y₁ is C.

The Preferred embodiments of formula 5-A
63. Z₁ is O, S, or N—R₁; Z₂, and Z₃ are independently CR₂; Z₄ is O, S, N—R₁, or CR₂; Y₁, and Y₂ are C; Y₄, and Y₃ are independently C, or N; W₁, W₂, W₃ are independently CR₄R₄; t=1 or 2.
64. Z₁ is O, S, or N—R₁; Z₂ and Z₃ are independently CR₂; Z₄ is O, S, N—R₁, or CR₂; Y₁, and Y₂ are C; Y₄, and Y₃ are independently C, or N; W₁, and W₃ are independently CR₄R₄; t=1 to 2; W₂ is O, S(O)r (r=0-2), N—R₁ or CR₄R₄.
65. Z₃ is N; Z₂ is CR₂; Z₁ is CR₂, or N; 4 is O, S, N—R₁, W₁, W₂, and W₃ are independently CR₄R₄; t=1 to 3; Y₁, Y₃, and Y₄ are C; Y₂ is N; one of Z₁, Z₂ or Z₄ is the carbon atom to which the remainder of the molecule is attached.
66. Z₁ is N; Z₂ is CR₂; Z₃ is CR₂, or N; Z₄ is O, S, or N—R₁; W₁, W₂, and W₃ are independently CR₄R₄; t=1 to 3; Y₂, Y₃, and Y₄ are C; Y₁ is N; one of Z₂, Z₃, Z₄ is the carbon atom to which the remainder of the molecule is attached.
67. Z₃ is N; Z₂ is CR₂; Z₄ is CR₂, or N; Z₄ is O, S, or N—R₁; Y₁, Y₃, and Y₄ are C; Y₂ is N; W₁, W₂, and W₃ are independently CR₄R₄, O, S(O)r (r=0-2), or N—R₁ with the proviso that no S—S, S—O or O—O bond formation can occur to form a saturated ring; t=1 to 3; one of Z₁, Z₂ or Z₄ is the carbon atom to which the remainder of the molecule is attached.
68. Z₁ is N; Z₂ is CR₂; Z₃ is CR₂, or N; Z₄ is O, S, or N—R₁; Y₂, Y₃, and Y₄ are C; Y₁ is N; W₁, W₂, and W₃ are independently CR₄R₄, O, S═(O)ᵣ (r=0-2); or N—R₁, with the proviso that no S—S, S—O or O—O bond formation can occur to form a saturated ring; t=1-3; one of Z₂, Z₃, Z₄ is a carbon atom to which the remainder of the molecule is attached.
69. Z₁ is CR₂; Z₂ is the carbon atom to which the remainder of the molecule is attached; Z₃ is N; Z₄ is O, S, or N—R₁; Y₁ is C; Y₂ is N; Y₃, and Y₄ are C; W₁, W₂, and W₃ are independently CR₄R₄, O, S═(O)ᵣ (r=0-2), or N—R₁ with the proviso that no S—S, S—O or O—O bond formation can occur to form a saturated ring; t=1 to 3.
70. Z₁ is the carbon atom to which the remainder of the molecule is attached; Z₂ is CR₂; Z₃ is N; Z₄ is O, S, N—R₁; Y₁ is C; Y₂ is N; Y₃, and Y₄ are C; W₁, W₂, and W₃ are independently CR₄R₄, O, S═(O)ᵣ (r=0-2), or N—R₁ with the proviso that no S—S, S═O or O—O bond formation can occur to form a saturated ring; t=1-3.
71. Z₁, Z₂, and Z₃ are independently CR₂, or N; Z₄ is CR₂; Y₁, Y₂ are C; Y₃, Y₄ are N; W₁, W₂, and W₃ are independently CR₄R₄, O, S═(O)r (r=0-2), or N—R₁, with the proviso that no S—S, S—O or O—O bond formation can occur to form a saturated ring; t=1-3.

The preferred embodiments of formula 5-B
72. Z₁, Z₂, Z₃, Z₄ are independently CR₂; Y₁, Y₂ are N; W₁, W₂ are independently O, S, N—R₁ or CR₄R₄; t=1-2.
73. Z₁, Z₂ are independently N, or CR₂; Z₃ is CR₂; Z₄ is O, S, or N—R₁; W₁, and W₂ are independently O, S, N—R₁, CR₄R₄; t=1-2.

The preferred embodiments of formula 6-A are:
74. Z₁ is O, S, N—R₁; Z₂, Z₃, Z₄, Z₅ are independently CR₂; W₁, W₂, W₃ are independently N—R₁, O, S═(O)ᵣ (r=0-2), or CR₄R₄; Y₁, Y₂ are C; t=1-3; one of Z₂, Z₃ is the carbon atom to which the remainder of the molecule is attached.
75. Z₁ is O, S, or N—R₁; Z₃ is N, O, or S; Z₂, Z₄, Z₅ are independently CR₂; W₁, W₂, W₃ are independently N—R₁, O, S═(O)ᵣ (r=0-2), or CR₄R₄; Y₁, and Y₂ are C; t=1-3; Z₂ is the carbon atom to which the remainder of the molecule is attached.
76. Z₁ is CR₂; Z₃ is N; Z₂, Z₄, and Z₅ are independently CR₂; W₁, W₂, and W₃ are independently N—R₁, O, S═(O)ᵣ (r=0-2), or CR₄R₄; Y₁ is N; Y₂ is C; t=1-3; Z₁, or Z₂ is the carbon atom to which the remainder of the molecule is attached.
77. Z₁ is N; Z₂, Z₃, Z₄, Z₅ are independently CR₂; W₁, W₂, W₃ are independently N—R₁, O, S═(O)ᵣ (r=0-2), or CR₄R₄; Y₁ is C; Y₂ is N; t=1-3; Z₂, or Z₃ is the carbon atom to which the remainder of the molecule is attached.
78. Z₁ is O, S, or N—R₁; Z₂ is N, O, or S; Z₃, Z₄, and Z₅ are independently CR₂; W₁, W₂, and W₃ are independently N—R₁, O, S═(O)ᵣ (r=0-2), or CR₄R₄; Y₁, and Y₂ are C; t=1-3; one of Z₃ is a carbon atom to which the remainder of the molecule is attached.
79. Z₁ is O, S, or N—R₁; Z₂, and Z₃ are independently CR₂; Z₁ and Z₅ are independently CR₂, or N; W₁, W₂, and W₃ are independently N—R₁, O, S═(O)ᵣ (r=0-2), or CR₄R₄; Y₁, Y₂ are C; t=1-3; Z₂ or Z₃ is the carbon atom to which the remainder of the molecule is attached.
80. Z₁ is O, S, or N—R₁; Z₃ is N, O, or S; Z₂ is CR₂; Z₄, Z₅ are independently CR₂, or N; W₁, W₂, W₃ are independently N—R₁, O, S═(O)ᵣ (r=0-2), or CR₄R₄; Y₁, Y₂ are C; t=1-3; Z₂ is the carbon atom to which the remainder of the molecule is attached.
81. Z₁ is CR₂; Z₃ is N; Z₂ is CR₂; Z₄, Z₅ are independently N, or CR₂; W₁, W₂, W₃ are independently N—R₁, O, S═(O)ᵣ (r=0-2), or CR₄R₄; Y₁ is N; Y₂ is C; t=1-3; Z₁ or Z₂ is the carbon atom to which the remainder of the molecule is attached.
82. Z₁ is N; Z₂, and Z₃ are independently CR₂; Z₄, Z₅ are independently N or CR₂; W₁, W₂, W₃ are independently N—R₁, O, S═(O)ᵣ (r=0-2), or CR₄R₄; Y₁ is C; Y₂ is N; t=1-3; Z₂ or Z₃ is the carbon atom to which the remainder of the molecule is attached.
83. Z₁ is O, S, or N—R₁; Z₂ is N, O, or S; Z₃ is CR₂; Z₁ and Z₅ are independently N, or CR₂; W₁, W₂, W₃ are independently N—R₁, O, S═(O)ᵣ (r=0-2), or CR₄R₄; Y₁, Y₂ are C; t=1-3; Z₃ is the carbon atom to which the remainder of the molecule is attached.

The preferred embodiments of formula 6-B are:
84. Z₁ is O, S, or N—R₁; Z₂, Z₃, Z₄, and Z₅ are independently CR₂; W₁, W₂, W₃ are independently N—R₁, O, S═(O)ᵣ (r=0-2), or CR₄R₄; Y₁, Y₂, Y₃, and Y₄ are C; t=1-3; Z₂ or Z₃ is the carbon atom to which the remainder of the molecule is attached.
85. Z₁ is O, S, or N—R₁; Z₃ is N, O, or S; Z₂, Z₄, Z₅ are independently CR₂; W₁, W₂, W₃ are independently N—R₁, O, S═(O)ᵣ (r=0-2), or CR₄R₄; Y₁, Y₂, Y₃, Y₄ are C; t=1-3; Z₂ is the carbon atom to which the remainder of the molecule is attached.
86. Z₁ is CR₂; Z₃ is N; Z₂, Z₁ and Z₅ are independently CR₂; W₁, W₂, W₃ are independently N—R₁, O, S═(O)ᵣ (r=0-2), or CR₄R₄; Y₃ is N; Y₁, Y₂, Y₄ are C; t=1-3; Z₁, or Z₂ is the carbon atom to which the remainder of the molecule is attached.
87. Z₁ is N; Z₂, Z₃, Z₄, Z₅ are independently CR₂; W₁, W₂, W₃ are independently N—R₁, O, S═(O)ᵣ (r=0-2), or CR₄R₄; Y₁, Y₂, Y₃ are C; Y₄ is N; t=1-3; Z₂ or Z₃ is the carbon atom to which the remainder of the molecule is attached.
88. Z₁ is O, S, or N—R₁; Z₂ is N, O, or S; Z₃, Z₄, and Z₅ are independently CR₂; W₁, W₂, W₃ are independently N—R₁, O, S═(O)ᵣ (r=0-2), or CR₄R₄; Y₁, Y₂, Y₃, and Y₄ are C; t=1-3; Z₃ is the carbon atom to which the remainder of the molecule is attached.
89. Z₁ is O, S, or N—R₁; Z₂, and Z₃ are independently CR₂; Z₄, and Z₅ are independently CR₂, or N; W₁, W₂, W₃ are independently N—R₁, O, S═(O)ᵣ (r=0-2), or CR₄R₄; Y₁, Y₂, Y₃, Y₄ are C; t=1-3; Z₂ or Z₃ is the carbon atom to which the remainder of the molecule is attached.
90. Z₁ is O, S, or N—R₁; Z₃ is N, O, or S; Z₂ is CR₂; Z₄, and Z₅ are independently CR₂ or N; W₁, W₂, W₃ are independently N—R₁, O, S═(O)ᵣ (r=0-2), or CR₄R₄; Y₁, Y₂, Y₃, Y₄ are C; t=1-3; Z₂ is the carbon atom to which the remainder of the molecule is attached.
91. Z₁ is CR₂; Z₃ is N; Z₂ is CR₂; Z₁ and Z₅ are independently N, CR₂; W₁, W₂, W₃ are independently N—R₁, O, S═(O)ᵣ (r=0-2), or CR₄R₄; Y₃ is N; Y₁, Y₂, Y₄ are C; t=1-3; Z₁ or Z₂ is the carbon atom to which the remainder of the molecule is attached.
92. Z₁ is N; Z₂, Z₃ are independently CR₂; Z₄, Z₅ are independently N, or CR₂; W₁, W₂, W₃ are independently N—R₁, O, S═(O)ᵣ (r=0-2), or CR₄R₄; Y₁, Y₂, Y₃ are C; Y₄ is N; t=1-3; Z₂, or Z₃ is the carbon atom to which the remainder of the molecule is attached.
93. Z₁ is O, S, or N—R₁; Z₂ is N, O, or S; Z₃ is CR₂; Z₄, Z₅ are independently N, or CR₂; W₁, W₂, W₃ are independently N—R₁, O, S═(O)ᵣ (r=0-2), or CR₄R₄; Y₁, Y₂, Y₃, Y₄ are C; t=1-3; Z₃ is the carbon atom to which the remainder of the molecule is attached.

The preferred embodiments of formula 6-C are:
94. Z₁, Z₃, Z₄, and Z₅ are independently N or CR₂; Z₂ is O, S, or N—R₁; Y₁, Y₂ are C; W₁, W₂ are independently N—R₁, O, S═(O)ᵣ (r=0-2), CR₄R₄; t=1-2.
95. Z₁, Z₃, Z₄, Z₅ are independently CR₂; Z₂ is O, S, or N—R₁; Y₁, Y₂ are C; W₁, W₂ are independently N—R₁, O, S═(O)ᵣ (r=0-2), or CR₄R₄; t=1-2.
96. Z₁, Z₃, Z₅ are independently CR₂; Z₂ is O, S, N—R₁; Z₄ is N; Y₁, Y₂ are C; W₁, W₂ are independently N—R₁, O, S═(O)ᵣ (r=0-2), or CR₄R₄; t=1-2.
97. Z₁, Z₂, Z₃, Z₁ and Z₅ are independently CR₂; Y₁ is C; Y₂ is N; W₁, W₂ are independently N—R₁, O, S═(O)ᵣ (r=0-2), or CR₄R₄; t=1-2.
98. Z₁, Z₂, Z₃, Z₅ are independently CR₂; Z₄ is N; Y₁ is C; Y₂ is N; W₁, W₂ are independently N—R₁, O, S═(O)ᵣ (r=0-2), or CR₄R₄; t=1-2.

The preferred embodiments of formula 7-A are:
99. Z₃, Z₆ are independently O, S, or N—R₁; Z₁, Z₂, Z₄, Z₅ are independently CR₂; W₁, W₂ are independently N—R₁, O, S═(O)ᵣ (r=0-2), or CR₄R₄; t=1-2.
100. Z₃, Z₆ are independently O, S, or N—R₁; Z₁, Z₄ are N; Z₂, Z₅ are independently CR₂; W₁, W₂ are independently N—R₁, O, S═(O)ᵣ (r=0-2), or CR₄R₄; t=1-2; Z₂ or Z₅ is the carbon atom to which the remainder of the molecule is attached.
101. Z₁, Z₄ are independently O, S, or N—R₁; Z₂, Z₃, Z₅, Z₆ are independently CR₂; W₁, W₂ are independently N—R₁, O, S═(O)ᵣ (r=0-2), or CR₄R₄; t=1-2.
102. Z₁, Z₄ are independently O, S, or N—R₁; Z₃, Z₆ are N; Z₂, Z₅ are independently CR₂; W₁, W₂ are independently N—R₁, O, S═(O)ᵣ (r=0-2), or CR₄R₄; t=1-2; Z₂ or Z₅ is the carbon atom to which the remainder of the molecule is attached.
103. Z₂, Z₅ are independently O, S, or N—R₁; Z₁, Z₃, Z₄, Z₆ are independently CR₂; W₁, W₂ are independently N—R₁, O, S═(O)ᵣ (r=0-2), or CR₄R₄; t=1-2.
104. Z₂, Z₅ are independently O, S, N—R₁; Z₁, Z₃, Z₄, Z₆ are independently CR₂, N, S; W₁, W₂ are independently N—R₁, O, S═(O)ᵣ (r=0-2), CR₄R₄; t=1-2; One of Z₁, Z₃, Z₄, Z₆ is the carbon atom to which the remainder of the molecule is attached.
105. Z₁ is CR₂, N; Z₂ is CR₂; Z₃ is N; Z₄, Z₅ are independently CR₂; Z₆ is N; Y₁, Y₃ are independently CR₂; Y₂, Y₄ are N; W₁, W₂ are independently N—R₁, O, S═(O)ᵣ (r=0-2), or CR₄R₄; t=1-2; One of Z₁, Z₂, Z₄, Z₅ is the carbon atom to which the remainder of the molecule is attached.
106. Z₁ is CR₂, or N; Z₂ is CR₂; Z₃ is O, S, N—R₁; Z₁, Z₅ are independently CR₂; Z₆ is N; Y₁, Y₄, Y₃ are independently CR₂; Y₂ is N; W₁, W₂ are independently N—R₁, O, S═(O)ᵣ (r=0-2), CR₄R₄; t=1-2; One of Z₁, Z₂, Z₄, Z₅ is the carbon atom to which the remainder of the molecule is attached.
107. Z₁ is CR₂, or N; Z₂ is CR₂; Z₃ is N; Z₄, Z₅, Z₆ are independently N, or CR₂; Y₁, and Y₃ are N; Y₂, Y₄ are independently CR₂; W₁, W₂ are independently N—R₁, O, S═(O)ᵣ (r=0-2), or CR₄R₄; t=1-2; One of Z₁, Z₂, Z₄, Z₅, Z₆ is the carbon atom to which the remainder of the molecule is attached.

The preferred embodiments of formula 7-B are:
108. Z₁, Z₂, Z₄, Z₅, Z₆ are independently CR₂; Z₃ is O, S, or N—R₁; Y₁, Y₂, Y₄ are C; Y₃ is N; W₁, W₂ are independently N—R₁, O, S═(O)ᵣ (r=0-2), or CR₄R₄; t=1-2.
109. Z₁, Z₂, Z₄, Z₅, Z₆ are independently CR₂ or N; Z₃ is O, S, or N—R₁; Y₁, Y₂, Y₄ are C; Y₃ is N; W₁, W₂ are independently N—R₁, O, S═(O)ᵣ (r=0-2), or CR₄R₄; t=1-2; One of Z₁, Z₂, Z₄, Z₅, Z₆ is the carbon atom to which the remainder of the molecule is attached.
110. Z₁, Z₂, Z₃, Z₅, Z₆ are independently CR₂; Z₄ is O, S, or N—R₁; Y₁, Y₂, Y₃ are C; Y₄ is N; W₁, W₂ are independently N—R₁, O, S═(O)ᵣ (r=0-2), or CR₄R₄; t=1-2; One of Z₁, Z₂, Z₃, Z₅, Z₆ is the carbon atom to which the remainder of the molecule is attached.
111. Z₁, Z₂, Z₃, Z₅, Z₆ are independently CR₂ or N; Z₄ is O, S, or N—R₁; Y₁, Y₂, Y₃ are C; Y₄ is N; W₁, W₂ are independently N—R₁, O, S═(O)ᵣ (r=0-2), or CR₄R₄; t=1-2; One of Z₁, Z₂, Z₃, Z₅, Z₆ is the carbon atom to which the remainder of the molecule is attached.
112. Z₁, Z₂, Z₃, Z₄, Z₅, Z₆ are independently CR₂; Y₁, Y₃ are N; Y₂, Y₄ are C; W₁, W₂ are independently N—R₁, O, S═(O)ᵣ (r=0-2), or CR₄R₄; t=1-2; One of Z₁, Z₂, Z₃, Z₄, Z₅, Z₆ is the carbon atom to which the remainder of the molecule is attached.
113. Z₁, Z₂, Z₃, Z₄, Z₅, Z₆ are independently CR₂ or N; Y₁, Y₃ are N; Y₂, Y₄ are C; W₁, W₂ are independently N—R₁, O, S═(O)ᵣ (r=0-2), or CR₄R₄; t=1-2; One of Z₁, Z₂, Z₃, Z₄, Z₅, Z₆ is the carbon atom to which the remainder of the molecule is attached.
114. Z₁, Z₃, Z₄, Z₅, Z₆ are independently CR₂; Z₂ is O, S, or N—R₁; Y₁, Y₂, Y₄ is C; Y₃ is N; W₁, W₂ are independently N—R₁, O, S═(O)ᵣ (r=0-2), or CR₄R₄; t=1-2; One of Z₁, Z₃, Z₄, Z₅, Z₆ is the carbon atom to which the remainder of the molecule is attached.
115. Z₁, Z₃, Z₄, Z₅, Z₆ are independently CR₂, or N; Z₂ is O, S, or N—R₁; Y₁, Y₂, Y₄ are C; Y₃=N; W₁, W₂ are independently N—R₁, O, S═(O)ᵣ (r=0-2), or CR₄R₄; t=1-2; One of Z₁, Z₃, Z₄, Z₅, Z₆ is the carbon atom to which the remainder of the molecule is attached.
116. Z₁, Z₂, Z₄, Z₆ are independently CR₂; Z₃, Z₅ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₆ is the carbon atom to which the remainder of the molecule is attached.
117. Z₁, Z₂, Z₄, Z₆ are independently CR₂, or N; Z₃, Z₅ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₆ is the carbon atom to which the remainder of the molecule is attached.
118. Z₁, Z₂, Z₄, Z₅ are independently CR₂; Z₃, Z₆ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅ is the carbon atom to which the remainder of the molecule is attached.
119. Z₁, Z₂, Z₄, Z₅ are independently CR₂, or N; Z₃, Z₆ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅ is the carbon atom to which the remainder of the molecule is attached.
120. Z₁, Z₂, Z₅, Z₆ are independently CR₂; Z₃, Z₄ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₅, Z₆ is the carbon atom to which the remainder of the molecule is attached.
121. Z₁, Z₂, Z₅, Z₆ are independently CR₂ or N; Z₃, Z₄ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₅, Z₆ is the carbon atom to which the remainder of the molecule is attached.
122. Z₁, Z₂, Z₄, Z₅ are independently CR₂; Z₃, Z₆ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₅, Z₆ is the carbon atom to which the remainder of the molecule is attached.
123. Z₁, Z₂, Z₄, Z₅ are independently CR₂, or N; Z₃, Z₆ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₅, Z₆ is the carbon atom to which the remainder of the molecule is attached.
124. Z₁, Z₂, Z₃, Z₄, Z₆ are independently CR₂; Z₅ is O, S, or N—R₁; Y₁ is N; Y₂, Y₃, Y₄ are C; W₁, W₂ are independently N—R₁, O, S═(O)ᵣ (r=0-2), or CR₄R₄; t=1-2; One of Z₁, Z₂, Z₃, Z₄, Z₆ is the carbon atom to which the remainder of the molecule is attached.
125. Z₁, Z₂, Z₃, Z₄, Z₆ are independently CR₂, or N; Z₅ is O, S, or N—R₁; Y₁ is N; Y₂, Y₃, Y₄ are C; W₁, W₂ are independently N—R₁, O, S═(O)ᵣ (r=0-2), or CR₄R₄; t=1-2; One of Z₁, Z₂, Z₃, Z₄, Z₆ is the carbon atom to which the remainder of the molecule is attached.
126. Z₁, Z₃, Z₄, Z₆ are independently CR₂; Z₂, Z₅ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₃, Z₄, Z₆ is the carbon atom to which the remainder of the molecule is attached.
127. Z₁, Z₃, Z₄, Z₆ are independently CR₂ or N; Z₂, Z₅ are independently O, S, or N—R₁; Y₁, Y₂, Y₃, Y₄ are C; W₁, W₂ are independently N—R₁, O, S═(O)ᵣ (r=0-2), or CR₄R₄; t=1-2; One of Z₁, Z₃, Z₄, Z₆ is the carbon atom to which the remainder of the molecule is attached.

The preferred embodiments of formula 8-A are:
128. Z₁ is O, S, or N—R₁; Z₂, Z₄, Z₅, Z₆, Z₇ are independently CR₂; Z₃ is CR₂, or N; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₆ is the carbon atom to which the remainder of the molecule is attached.
129. Z₁ is O, S, or N—R₁; Z₂, Z₄, Z₅, Z₆, Z₇ are independently CR₂; Z₃ is CR₂; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
130. Z₁ is O, S, or N—R₁; Z₂, Z₄, Z₅, Z₆, Z₇ are independently CR₂; Z₃ is N; Y₁, Y₂, Y₃, Y₄ are C; W₁, W₂ are independently N—R₁, O, S═(O)ᵣ (r=0-2), or CR₄R₄; t=1-2; One of Z₂, Z₄, Z₅, Z₆, Z₇ is the carbon atom to which the remainder of the molecule is attached.
131. Z₁ is O, S, or N—R₁; Z₃, Z₄, Z₅, Z₆, Z₇ are independently CR₂; Z₂ is CR₂, or N; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
132. Z₁ is O, S, or N—R₁; Z₃, Z₄, Z₅, Z₆, Z₇ are independently CR₂; Z₂ is N; Y₁, Y₂, Y₃, Y₄ are C; W₁, W₂ are independently N—R₁, O, S═(O)ᵣ (r=0-2), or CR₄R₄; t=1-2; One of Z₃, Z₄, Z₅, Z₆, Z₇ is the carbon atom to which the remainder of the molecule is attached.
133. Z₁ is O, S, or N—R₁; Z₃, Z₄, Z₅, Z₆, Z₇ are independently CR₂; Z₂ is CR₂; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
134. Z₁ is O, S, or N—R₁; Z₂, Z₃, Z₄, Z₅, Z₆, Z₇ are independently N or CR₂; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
135. Z₁ is O, S, or N—R₁; Z₂ is CR₂; Z₄, Z₅, Z₆, Z₇ are independently N or CR₂; Z₃ is CR₂; Y₁, Y₂, Y₃, Y₄ are C; W₁, W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
136. Z₁ is O, S, or N—R₁; Z₂ is CR₂; Z₄, Z₅, Z₆, and Z₇ are independently N or CR₂; Z₃ is N; Y₁, Y₂, Y₃, Y₄ are C; W₁, and W₂ are independently N—R₁, O, S═(O)ᵣ (r=0-2), or CR₄R₄; t=1-2; One of Z₂, Z₄, Z₅, Z₆, Z₇ is the carbon atom to which the remainder of the molecule is attached.
137. Z₁ is O, S, or N—R₁; Z₄, Z₅, Z₆, and Z₇ are independently N or CR₂; Z₂ and Z₃ are independently CR₂ or N; Y₁, Y₂, Y₃, Y₄ are C; W₁ and W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
138. Z₁ is O, S, or N—R₁; Z₃ is CR₂; Z₄, Z₅, Z₆, and Z₇ are independently N or CR₂; Z₂ is N; Y₁, Y₂, Y₃, and Y₄ are C; W₁ and W₂ are independently N—R₁, O, S═(O)ᵣ (r=0-2), or CR₄R₄; t=1-2; One of Z₃, Z₄, Z₅, Z₆, Z₇ is the carbon atom to which the remainder of the molecule is attached.
139. Z₁ is O, S, N—R₁; Z₄, Z₅, Z₆, and Z₇ are independently N or CR₂; Z₂ and Z₃ are independently CR₂; Y₁, Y₂, Y₃, Y₄ are C; W₁ and W₂ are independently N—R₁, O, S═(O)ᵣ (r=0-2), or CR₄R₄; t=1-2; One of Z₂, Z₃, Z₄, Z₅, Z₆, Z₇ is the carbon atom to which the remainder of the molecule is attached.
140. Z₃ is O, S, or N—R₁; Z₂, Z₄, Z₅, Z₆, and Z₇ are independently CR₂; Z₁ is CR₂ or N; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
141. Z₃ is O, S, or N—R₁; Z₁, Z₂, Z₄, Z₅, Z₇ are independently CR₂; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
142. Z₃ is O, S, or N—R₁; Z₂, Z₄, Z₅, Z₆, and Z₇ are independently CR₂; Z₁ is N; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
143. Z₃ is O, S, or N—R₁; Z₁, Z₄, Z₅, Z₆, Z₇ are independently CR₂; Z₂ is CR₂ or N; Y₁, Y₂, Y₃, Y₄ are C; W₁, W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
144. Z₃ is O, S, or N—R₁; Z₁, Z₄, Z₅, Z₆, Z₇ are independently CR₂; Z₂ is N; Y₁, Y₂, Y₃, Y₄ are C; W₁ and W₂ are independently N—R₁, O, S═(O)ᵣ (r=0-2), or CR₄R₄; t=1-2; One of Z₁, Z₄, Z₅, Z₆, Z₇ is the carbon atom to which the remainder of the molecule is attached.
145. Z₃ is O, S, or N—R₁; Z₁, Z₂, Z₄, Z₅, Z₆, Z₇ are independently CR₂; Y₁, Y₂, Y₃, Y₄ are C; W₁ and W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
146. Z₃ is O, S, or N—R₁; Z₂, Z₄, Z₅, Z₆, Z₇ are independently N or CR₂; Z₁ is CR₂, or N; Y₁, Y₂, Y₃, Y₄ are C; W₁, W₂ are independently N—R₁, O, S═(O)ᵣ (r=0-2), CR₄R₄; t=1-2; One of Z₁, Z₂, Z₄, Z₅, Z₆, Z₇ is the carbon atom to which the remainder of the molecule is attached.
147. Z₃ is O, S, or N—R₁; Z₂ is CR₂; Z₄, Z₅, Z₆, Z₇ are independently N or CR₂; Z₁ is CR₂; Y₁, Y₂, Y₃, Y₄ are C; W₁ and W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
148. Z₃ is O, S, or N—R₁; Z₂ is CR₂; Z₄, Z₅, Z₆, and Z₇ are independently N or CR₂; Z₁ is N; Y₁, Y₂, Y₃, Y₄ are C; W₁ and W₂ are independently N—R₁, O, S═(O)ᵣ (r=0-2), or CR₄R₄; t=1-2; One of Z₂, Z₄, Z₅, Z₆, Z₇ is the carbon atom to which the remainder of the molecule is attached.
149. Z₃ is O, S, or N—R₁; Z₄, Z₅, Z₆, and Z₇ are independently N or CR₂; Z₂ and Z₁ are independently CR₂ or N; Y₁, Y₂, Y₃, Y₄ are C; W₁ and W₂ are independently N—R₁, O, S═(O)ᵣ (r=0-2), or CR₄R₄; t=1-2; One of Z₁, Z₂, Z₄, Z₅, Z₆, Z₇ is the carbon atom to which the remainder of the molecule is attached.
150. Z₃ is O, S, N—R₁; Z₁ is CR₂; Z₄, Z₅, Z₆, Z₇ are independently N or CR₂; Z₂ is N; Y₁, Y₂, Y₃, Y₄ are C; W₁, W₂ are independently N—R₁, O, S═(O)ᵣ (r=0-2), CR₄R₄; t=1-2; One of Z₁, Z₄, Z₅, Z₆, and Z₇ is the carbon atom to which the remainder of the molecule is attached.
151. Z₃ is O, S, N—R₁; Z₄, Z₅, Z₆, and Z₇ are independently N or CR₂; Z₁ and Z₂ are independently CR₂; Y₁, Y₂, Y₃, Y₄ are C; W₁ and W₂ are independently N—R₁, O, S═(O)ᵣ (r=0-2), or CR₄R₄; t=1-2; One of Z₁, Z₂, Z₄, Z₅, Z₆, and Z₇ is the carbon atom to which the remainder of the molecule is attached.
152. Z₁, Z₂, Z₃, Z₄, Z₅, Z₆, and Z₇ are independently CR₂; Y₄ is N; Y₁, Y₂, Y₃ are C; W₁, W₂ are independently N—R₁, O, S═(O)ᵣ (r=0-2), or CR₄R₄; t=1-2; One of Z₁, Z₂, Z₃, Z₄, Z₅, Z₆, Z₇ is the carbon atom to which the remainder of the molecule is attached.
153. Z₁ is N; Z₂, Z₃, Z₄, Z₅, Z₆, and Z₇ are independently CR₂; Y₄ is N; Y₁, Y₂, Y₃ are C; W₁ and W₂ are independently N—R₁, O, S═(O)ᵣ (r=0-2), or CR₄R₄; t=1-2; One of Z₂, Z₃, Z₄, Z₅, Z₆, and Z₇ is the carbon atom to which the remainder of the molecule is attached.
154. Z₂ is N; Z₁, Z₃, Z₄, Z₅, Z₆, Z₇ are independently CR₂; Y₄ is N; Y₁, Y₂, Y₃ are C; W₁ and W₂ are independently N—R₁, O, S═(O)ᵣ (r=0-2), CR₄R₄; t=1-2; One of Z₁, Z₃, Z₄, Z₅, Z₆, and Z₇ is the carbon atom to which the remainder of the molecule is attached.
155. Z₃ is N; Z₁, Z₂, Z₄, Z₅, Z₆, and Z₇ are independently CR₂; Y₄ is N; Y₁, Y₂, Y₃ are C; W₁, W₂ are independently N—R₁, O, S═(O)ᵣ (r=0-2), or CR₄R₄; t=1-2; One of Z₁, Z₂, Z₄, Z₅, Z₆, and Z₇ is the carbon atom to which the remainder of the molecule is attached.
156. Z₁ and Z₂ are N; Z₃, Z₄, Z₅, Z₆, Z₇ are independently CR₂; Y₄ is N; Y₁, Y₂, Y₃ are C; W₁, W₂ are independently N—R₁, O, S═(O)ᵣ (r=0-2), CR₄R₄; t=1-2; One of Z₃, Z₄, Z₅, Z₆, Z₇ is the carbon atom to which the remainder of the molecule is attached.
157. Z₁, Z₃ are N; Z₂, Z₄, Z₅, Z₆, Z₇ are independently CR₂; Y₄ is N; Y₁, Y₂, Y₃ are C; W₁, W₂ are independently N—R₁, O, S═(O)ᵣ (r=0-2), CR₄R₄; t=1-2; One of Z₁, Z₂, Z₃, Z₄, Z₅, Z₆, Z₇ is the carbon atom to which the remainder of the molecule is attached.
158. Z₁, Z₂, Z₃ are N; Z₄, Z₅, Z₆, Z₇ are independently CR₂; Y₄ is N; Y₁, Y₂, Y₃ are C; W₁, W₂ are independently N—R₁, O, S═(O)ᵣ (r=0-2), CR₄R₄; t=1-2; One of Z₄, Z₅, Z₆, Z₇ is the carbon atom to which the remainder of the molecule is attached.
159. Z₁, Z₂, Z₃ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₁, Z₂, Z₃, Z₄, Z₅, Z₆, Z₇ is the carbon atom to which the remainder of the molecule is attached.
160. Z₁ is N; Z₂, Z₃ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₂, Z₃, Z₄, Z₅, Z₆, Z₇ is the carbon atom to which the remainder of the molecule is attached.
161. Z₂ is N; Z₁, Z₃ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₁, Z₃, Z₄, Z₅, Z₆, Z₇ is the carbon atom to which the remainder of the molecule is attached.
162. Z₃ is N; Z₁, Z₂ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₁, Z₂, Z₄, Z₅, Z₆, Z₇ is the carbon atom to which the remainder of the molecule is attached.
163. Z₁, Z₂ are N; Z₃ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₃, Z₄, Z₅, Z₆, Z₇ is the carbon atom to which the remainder of the molecule is attached.
164. Z₁, Z₃ are N; Z₂ are independently CR₂; Z₄, Z₅, Z₆, Z₇ are independently N, CR₂; Y₄ is N; Y₁, Y₂, Y₃ are C; W₁, W₂ are independently N—R₁, O, S═(O)ᵣ (r=0-2), CR₄R₄; t=1-2; One of Z₁, Z₂, Z₃, Z₄, Z₅, Z₆, Z₇ is the carbon atom to which the remainder of the molecule is attached.
165. Z₁, Z₂, Z₃ are N; Z₄, Z₅, Z₆, Z₇, are independently N, CR₂; Y₄ is N; Y₁, Y₂, Y₃ are C; W₁, W₂ are independently N—R₁, O, S═(O)ᵣ (r=0-2), CR₄R₄; t=1-2; One of 4, Z₅, Z₆, Z₇ is the carbon atom to which the remainder of the molecule is attached.

The preferred embodiments of formula 8-B are:
166. Z₁ is O, S, N—R₁; Z₂, Z₃, Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₂, Z₃, Z₄, Z₅, Z₆, Z₇ is the carbon atom to which the remainder of the molecule is attached.
167. Z₁ is O, S, N—R₁; Z₂, Z₃, Z₄, Z₅, Z₆, Z₇ are CH₂; Y₁, Y₂, Y₃, Y₄ are C; W₁, W₂ are independently N—R₁, O, S═(O)ᵣ (r=0-2), CR₄R₄; t=0-2; One of Z₂, Z₃, Z₄, Z₅, Z₆, Z₇ is the carbon atom to which the remainder of the molecule is attached.
168. Z₁ is O, S, N—R₁; Z₂ is N; Z₃, Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₃, Z₄, Z₅, Z₆, Z₇ is the carbon atom to which the remainder of the molecule is attached.
169. Z₁ is O, S, N—R₁; Z₂, Z₃ are N; Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₄, Z₅, Z₆, Z₇ is the carbon atom to which the remainder of the molecule is attached.
170. Z₁ is O, S, N—R₁; Z₂, Z₃, Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₃ is N; Z₂, Z₄, Z₅, Z₆, Z₇ is the carbon atom to which the remainder of the molecule is attached.
171. Z₂ is O, S, N—R₁; Z₁, Z₃, Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₁, Z₃, Z₄, Z₅, Z₆, Z₇ is the carbon atom to which the remainder of the molecule is attached.
172. Z₂ is O, S, N—R₁; Z₁, Z₃, Z₄, Z₅, Z₆, Z₇ are independently CR₂; Y₁, Y₂, Y₃, Y₄ are C; W₁, W₂ are independently N—R₁, O, S═(O)ᵣ (r=0-2), CR₄R₄; t=0-2; One of Z₁, Z₃, Z₄, Z₅, Z₆, Z₇ is the carbon atom to which the remainder of the molecule is attached.
173. Z₂ is O, S, N—R₁; Z₁ is N; Z₃ is CR₂; Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₃, Z₄, Z₅, Z₆, Z₇ is the carbon atom to which the remainder of the molecule is attached.
174. Z₂ is O, S, N—R₁; Z₁, Z₃ are N; Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₄, Z₅, Z₆, Z₇ is the carbon atom to which the remainder of the molecule is attached.
175. Z₃ is O, S, N—R₁; Z₁, Z₂, Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₁, Z₂, Z₄, Z₅, Z₆, Z₇ is the carbon atom to which the remainder of the molecule is attached.
176. Z₃ is O, S, N—R₁; Z₁, Z₂, Z₄, Z₅, Z₆, Z₇ are independently CR₂; Y₁, Y₂, Y₃, Y₄ are C; W₁, W₂ are independently N—R₁, O, S═(O)ᵣ (r=0-2), CR₄R₄; t=0-2; One of Z₁, Z₂, Z₄, Z₅, Z₆, Z₇ is the carbon atom to which the remainder of the molecule is attached.
177. Z₃ is O, S, N—R₁; Z₁ is N; Z₂ is CR₂; Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of Z₂, Z₄, Z₅, Z₆, Z₇ is the carbon atom to which the remainder of the molecule is attached.
178. Z₃ is O, S, N—R₁; Z₁, Z₂ is N; Z₄, Z₅, Z₆, Z₇ are independently N, CR₂; Y₁, Y₂, Y₃, Y₄ are C; W₁, W₂ are independently N—R₁, O, S═(O)ᵣ (r=0-2), CR₄R₄; t=0-2; One of 4, Z₅, Z₆, Z₇ is the carbon atom to which the remainder of the molecule is attached.
179. Z₁, Z₂, Z₃, Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₁, Z₂, Z₃, Z₄, Z₅, Z₆, Z₇ is the carbon atom to which the remainder of the molecule is attached.
180. Z₁ is N; Z₂, Z₃ are independently CR₂; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₂, Z₃, Z₄, Z₅, Z₆, Z₇ is the carbon atom to which the remainder of the molecule is attached.
181. Z₁, Z₂ are N; Z₃ is CR₂; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₃, Z₄, Z₅, Z₆, Z₇ is the carbon atom to which the remainder of the molecule is attached.
182. Z₁, Z₂, Z₃ are N; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₄, Z₅, Z₆, Z₇ is the carbon atom to which the remainder of the molecule is attached.
183. Z₂ is N; Z₁, Z₃ are independently CR₂; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₁, Z₃, Z₄, Z₅, Z₆, Z₇ is the carbon atom to which the remainder of the molecule is attached.
184. Z₂, Z₃ are N; Z₁ is CR₂; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₁, Z₄, Z₅, Z₆, Z₇ is the carbon atom to which the remainder of the molecule is attached.
185. Z₃ is N; Z₁, Z₂ are independently CR₂; Z₄, Z₅, Z₆, Z₇ are independently N, CR₂; Y₁ is N; Y₂, Y₃, Y₄ are C; W₁, W₂ are independently N—R₁, O, S═(O)ᵣ (r=0-2), CR₄R₄; t=0-2; One of Z₁, Z₂, Z₄, Z₅, Z₆, Z₇ is the carbon atom to which the remainder of the molecule is attached.
186. Z₁, Z₂, Z₃, Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₁, Z₂, Z₃, Z₄, Z₅, Z₆, Z₇ is the carbon atom to which the remainder of the molecule is attached.
187. Z₁ is N; Z₂, Z₃ are independently CR₂; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₂, Z₃, Z₄, Z₅, Z₆, Z₇ is the carbon atom to which the remainder of the molecule is attached.
188. Z₁, Z₂ are N; Z₃ is CR₂; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₃, Z₄, Z₅, Z₆, Z₇ is the carbon atom to which the remainder of the molecule is attached.
189. Z₁, Z₂, Z₃ are N; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₄, Z₅, Z₆, Z₇ is the carbon atom to which the remainder of the molecule is attached.
190. Z₂ is N; Z₁, Z₃ are independently CR₂; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₁, Z₃, Z₅, Z₅, Z₆, Z₇ is the carbon atom to which the remainder of the molecule is attached.
191. Z₂, Z₃ are N; Z₁ is CR₂; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₁, Z₄, Z₅, Z₆, Z₇ is the carbon atom to which the remainder of the molecule is attached.
192. Z₃ is N; Z₁, Z₂ are independently CR₂; Z₄, Z₅, Z₆, Z₇ are independently N, CR₂; Y₄ is N; Y₂, Y₃, Y₁ are C; W₁, W₂ are independently N—R₁, O, S═(O)ᵣ (r=0-2), CR₄R₄; t=0-2; One of Z₁, Z₂, Z₄, Z₅, Z₆, Z₇ is the carbon atom to which the remainder of the molecule is attached.

The preferred embodiments of formula 9-A are:
193. Z₁ is O, S, N—R₁; Z₂, Z₃ are independently CR₂; Y₁, Y₄ are C; Y₂, Y₃ are independently N, C, CH (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
194. Z₁ is O, S, N—R₁; Z₂ is N; Z₃ is CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
195. Z₁ is O, S, N—R₁; Z₃ is N Z₂ is CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
196. Z₂ is O, S, N—R₁; Z₁, Z₃ are independently CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
197. Z₂ is O, S, N—R₁; Z₁ is N; Z₃ is CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
198. Z₂ is O, S, N—R₁; Z₃ is N Z₁ is CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₁ is the carbon atom to which the remainder of the molecule is attached.
199. Z₃ is O, S, N—R₁; Z₁, Z₂ are independently CR₂; Y₁, Y₄ are C; Y₂, Y₃ are independently C, CH, N (in between a double bond might be present); W₁, W₂, W₃, W₄, Ws are independently N—R₁, O, S═(O)ᵣ (r=0-2), CR₄R₄; t=0-2; u=1-3; One of Z₁, Z₂ is the carbon atom to which the remainder of the molecule is attached.
200. Z₃ is O, S, N—R₁; Z₁ is N; Z₂ is CR₂; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
201. Z₃ is O, S, N—R₁; Z₁ is CH₂; Z₂ is N; Y₁, Y₄ are C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₁ is the carbon atom to which the remainder of the molecule is attached.
202. Z₁, Z₂, Z₃ are independently CR₂; Y₁ is N; Y₄ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₁, Z₂, Z₃ is the carbon atom to which the remainder of the molecule is attached.
203. Z₁ is N; Z₂, Z₃ are independently CR₂; Y₁ is N; Y₄ is C; Y₂, Y₃ are independently C, CH. N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
204. Z₁, Z₂ is N; Z₃ is CR₂; Y₁ is N; Y₄ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
205. Z₁, Z₃ are N; Z₂ is CR₂; Y₁ is N; Y₄ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
206. Z₁, Z₂, Z₃ are independently CR₂; Y₄ is N; Y₁ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₁, Z₂, Z₃ is the carbon atom to which the remainder of the molecule is attached.
207. Z₁ is N; Z₂, Z₃ are independently CR₂; Y₄ is N; Y₁ is C; Y₂, Y₃ are independently C, CH. N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
208. Z₁, Z₂ are N; Z₃ is CR₂; Y₄ is N; Y₁ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
209. Z₁, Z₃ are N; Z₂ is CR₂; Y₄ is N; Y₁ is C; Y₂, Y₃ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.

The preferred embodiments of formula 9-B:
210. Z₁ is O, S, N—R₁; Z₂, Z₃ are independently CR₂; Y₁, Y₂ are C; Y₃, Y₄ are independently N, C, CH (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
211. Z₁ is O, S, N—R₁; Z₂ is N; Z₃ is CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
212. Z₁ is O, S, N—R₁; Z₃ is N Z₂ is CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
213. Z₂ is O, S, N—R₁; Z₁, Z₃ are independently CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
214. Z₂ is O, S, N—R₁; Z₁ is N; Z₃ is CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
215. Z₂ is O, S, N—R₁; Z₃ is N Z₁ is CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₁ is the carbon atom to which the remainder of the molecule is attached.
216. Z₃ is O, S, N—R₁; Z₁, Z₂ are independently CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₁, Z₂ is the carbon atom to which the remainder of the molecule is attached.
217. Z₃ is O, S, N—R₁; Z₁ is N; Z₂ is CR₂; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
218. Z₃ is O, S, N—R₁; Z₁ is CH₂; Z₂ is N; Y₁, Y₂ are C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₁ is the carbon atom to which the remainder of the molecule is attached.
219. Z₁, Z₂, Z₃ are independently CR₂; Y₁ is N; Y₂ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₁, Z₂, Z₃ is the carbon atom to which the remainder of the molecule is attached.
220. Z₁ is N; Z₂, Z₃ are CR₂; Y₁ is N; Y₂ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
221. Z₁, Z₂ are N; Z₃ is CR₂; Y₁ is N; Y₂ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
222. Z₁, Z₃ are N; Z₂ is CR₂; Y₁ is N; Y₂ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.
223. Z₁, Z₂, Z₃ are independently CR₂; Y₂ is N; Y₁ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; One of Z₁, Z₂, Z₃ is the carbon atom to which the remainder of the molecule is attached.
224. Z₁ is N; Z₂, Z₃ are independently CR₂; Y₂ is N; Y₁ is C; Y₃, Y₄ are independently C, CH. N (in between a double bond might be present); W₁, W₂, W₃, W₄, W₅ are independently N—R₁, O, S═(O)ᵣ (r=0-2), CR₄R₄; t=0-2; u=1-3; One of Z₂, Z₃ is the carbon atom to which the remainder of the molecule is attached.
225. Z₁, Z₂ are N; Z₃ is CR₂; Y₂ is N; Y₁ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₃ is the carbon atom to which the remainder of the molecule is attached.
226. Z₁, Z₃ are N; Z₂ is CR₂; Y₂ is N; Y₁ is C; Y₃, Y₄ are independently C, CH, N (in between a double bond might be present); W₁, W₂, W₃, W₄, W₅ are independently N—R₁, O, S═(O)ᵣ (r=0-2), CR₄R₄; t=0-2; u=1-3; Z₂ is the carbon atom to which the remainder of the molecule is attached.

The preferred embodiments of formula 10-A:
227. Z₁, Z₂, Z₃, Z₄, Z₆, Z₇, Z₈, Z₉ are independently CR₂; Z₅ is O, S, N—R₁; Y₁, Y₂, Y₃, Y₄ are C; one of Z₁, Z₂, Z₃, Z₄, Z₆, Z₇, Z₈, Z₉ must be a carbon atom to which the remainder of the molecule is attached.
228. Z₁ is N; Z₂, Z₃, Z₄, Z₆, Z₇, Z₈, Z₉ are independently CR₂; Z₅ is O, S, N—R₁; Y₁, Y₂, Y₃, Y₄ are C; one of Z₂, Z₃, Z₄, Z₆, Z₇, Z₈, Z₉ must be a carbon atom to which the remainder of the molecule is attached.
229. Z₁, Z₂ are N; Z₃, Z₄, Z₆, Z₇, Z₈, Z₉ are independently CR₂; Z₅ is O, S, N—R₁; Y₁, Y₂, Y₃, Y₄ are C; one of Z₃, Z₄, Z₆, Z₇, Z₈, Z₉ must be a carbon atom to which the remainder of the molecule is attached.
230. Z₁, Z₃ are N; Z₂, Z₄, Z₆, Z₇, Z₈, Z₉ are independently CR₂; Z₅ is O, S, N—R₁; Y₁, Y₂, Y₃, Y₄ are C; one of Z₂, Z₄, Z₆, Z₇, Z₈, Z₉ must be a carbon atom to which the remainder of the molecule is attached.
231. Z₁, Z₄ are N; Z₃, Z₆, Z₇, Z₈, Z₉ are independently CR₂; Z₅ is O, S, N—R₁; Y₁, Y₂, Y₃, Y₄ are C; one of Z₃, Z₆, Z₇, Z₈, Z₉ must be a carbon atom to which the remainder of the molecule is attached.
232. Z₁ is N; Z₂, Z₃, Z₄ are independently CR₂; Z₆, Z₇, Z₈, Z₉ are independently N, CR₂; Z₅ is O, S, N—R₁; Y₁, Y₂, Y₃, Y₄ are C; one of Z₂, Z₃, Z₄, Z₆, Z₇, Z₈, Z₉ must be a carbon atom to which the remainder of the molecule is attached.
233. Z₁, Z₂ are N; Z₃, Z₄ are independently CR₂; Z₆, Z₇, Z₈, Z₉ are independently N, CR₂; Z₅ is O, S, N—R₁; Y₁, Y₂, Y₃, Y₄ are C; one of Z₃, Z₄, Z₆, Z₇, Z₈, Z₉ must be a carbon atom to which the remainder of the molecule is attached.
234. Z₁, Z₃ are N; Z₂, Z₄ are independently CR₂; Z₆, Z₇, Z₈, Z₉ are independently N, CR₂; Z₅ is O, S, N—R₁; Y₁, Y₂, Y₃, Y₄ are C; one of Z₂, Z₄, Z₆, Z₇, Z₈, Z₉ must be a carbon atom to which the remainder of the molecule is attached.
235. Z₁, Z₄ are N; Z₂, Z₃ are independently CR₂; Z₆, Z₇, Z₈, Z₉ are independently N, CR₂; Z₅ is O, S, N—R₁; Y₁, Y₂, Y₃, Y₄ are C; one of Z₂, Z₃, Z₆, Z₇, Z₈, Z₉ must be a carbon atom to which the remainder of the molecule is attached.
236. Z₂ is N; Z₁, Z₃, Z₄, Z₆, Z₇, Z₈, Z₉ are independently CR₂; Z₅ is O, S, N—R₁; Y₁, Y₂, Y₃, Y₄ are C; one of Z₁, Z₃, Z₄, Z₆, Z₇, Z₈, Z₉ must be a carbon atom to which the remainder of the molecule is attached.
237. Z₂, Z₃ are N; Z₁, Z₄, Z₆, Z₇, Z₈, Z₉ are independently CR₂; Z₅ is O, S, N—R₁; Y₁, Y₂, Y₃, Y₄ are C; one of Z₁, Z₄, Z₆, Z₇, Z₈, Z₉ must be a carbon atom to which the remainder of the molecule is attached.
238. Z₂, Z₄ are N; Z₁, Z₃, Z₆, Z₇, Z₈, Z₉ are independently CR₂; Z₅ is O, S, N—R₁; Y₁, Y₂, Y₃, Y₄ are C; one of Z₁, Z₃, Z₆, Z₇, Z₈, Z₉ must be a carbon atom to which the remainder of the molecule is attached.
239. Z₂ is N; Z₁, Z₃, Z₄ are independently CR₂; Z₆, Z₇, Z₈, Z₉ are independently N, CR₂; Z₅ is O, S, N—R₁; Y₁, Y₂, Y₃, Y₄ are C; one of Z₁, Z₃, Z₄, Z₆, Z₇, Z₈, Z₉ must be a carbon atom to which the remainder of the molecule is attached.
240. Z₂, Z₃ are N; Z₁, Z₄ are independently CR₂; Z₆, Z₇, Z₈, Z₉ are independently N, CR₂; Z₅ is O, S, N—R₁; Y₁, Y₂, Y₃, Y₄ are C; one of Z₁, Z₄, Z₆, Z₇, Z₈, Z₉ must be a carbon atom to which the remainder of the molecule is attached.
241. Z₂, Z₄ are N; Z₁, Z₃ are independently CR₂; Z₆, Z₇, Z₈, Z₉ are independently N, CR₂; Z₅ is O, S, N—R₁; Y₁, Y₂, Y₃, Y₄ are C; one of Z₁, Z₃, Z₆, Z₇, Z₈, Z₉ must be a carbon atom to which the remainder of the molecule is attached.

The preferred embodiments of formula 11-A:
242. Z₁, Z₂, Z₃, Z₄, Z₆, Z₇, Z₈, Z₉ are independently CR₂; Z₅, Z₁₀ are independently O, S, N—R₁; Y₁, Y₂, Y₃, Y₄ are C; Any one of Z₁, Z₂, Z₃, Z₄, Z₆, Z₇, Z₈, Z₉ must be a carbon atom to which the remainder of the molecule is attached.
243. Z₁, Z₂, Z₃, Z₄, Z₆, Z₇, Z₈, Z₉ are independently N, CR₂; Z₅, Z₁₀ are independently O, S, N—R₁; Y₁, Y₂, Y₃, Y₄ are C; Any one of Z₁, Z₂, Z₃, Z₄, Z₆, Z₇, Z₈, Z₉ must be a carbon atom to which the remainder of the molecule is attached.

The preferred embodiments of formula 11-B:
244. Z₁, Z₂, Z₃, Z₄, Z₇, Z₈, Z₉, Z₁₀ are independently CR₂; Z₅, Z₆ are independently N, CR₂; Y₁, Y₂, Y₃, Y₄ are C; Any one of Z₁, Z₂, Z₃, Z₄, Z₇, Z₈, Z₉, Z₁₀ must be a carbon atom to which the remainder of the molecule is attached.
245. Z₁, Z₂, Z₃, Z₄ are independently CR₂, N; Z₅, Z₆, Z₇, Z₈, Z₉, Z₁₀ are independently CR₂; Y₁, Y₂, Y₃, Y₄ are C; Any one of Z₁, Z₂, Z₃, Z₄, Z₆, Z₇, Z₈, Z₉, Z₁₀ must be a carbon atom to which the remainder of the molecule is attached.

The preferred embodiments of formula 11-C:
246. Z₁, Z₂, Z₃, Z₄, Z₆, Z₇, Z₈, Z₉ are independently CR₂; Y₁ is N; Y₂ is C; Any one of Z₁, Z₂, Z₃, Z₄, Z₆, Z₇, Z₈, Z₉ must be a carbon atom to which the remainder of the molecule is attached.
247. Z₁, Z₂, Z₃, Z₄, Z₇, Z₈, Z₉ are independently CR₂; Z₆ is O, S, N—R₁; Y₁ is C; Y₂ is N; Any one of Z₁, Z₂, Z₃, Z₄, Z₆, Z₇, Z₈, Z₉ must be a carbon atom to which the remainder of the molecule is attached.

The preferred embodiments of formula 12-A:
248. Z₁, Z₂, Z₃, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₂, Z₃, Z₄ must be a carbon atom to which the remainder of the molecule is attached.
249. Z₁, Z₂, Z₃, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by Wt, W₂, W₃ one double bond might be present; t=1-4; Y₁, Y₂, Y₃ are C; Y₄ is N; Any one of Z₁, Z₂, Z₃, Z₄ must be a carbon atom to which the remainder of the molecule is attached.
250. Z₁, Z₂, Z₃, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₂, Z₃, Z₄ must be a carbon atom to which the remainder of the molecule is attached.
251. Z₁ is N; Z₂, Z₃, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₂, Z₃, Z₄ must be a carbon atom to which the remainder of the molecule is attached.
252. Z₁, Z₂ are N; Z₃, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₃, Z₄ must be a carbon atom to which the remainder of the molecule is attached.
253. Z₁, Z₃ are N; Z₂, Z₄ are independently CR₂; Z₅ is O, S, N—R₁; Z₁, Z₂ are N; Z₃, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₂, Z₄ must be a carbon atom to which the remainder of the molecule is attached.
254. Z₁, Z₄ are N; Z₂, Z₃ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₂, Z₃ must be a carbon atom to which the remainder of the molecule is attached.
255. Z₁ is N; Z₂, Z₃, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₂, Z₃, Z₄ must be a carbon atom to which the remainder of the molecule is attached.
256. Z₁, Z₃ are N; Z₂, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₂, Z₄ must be a carbon atom to which the remainder of the molecule is attached.
257. Z₁, Z₄ are N; Z₂, Z₃ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₂, Z₃ must be a carbon atom to which the remainder of the molecule is attached.
258. Z₁, Z₂ are N; Z₃, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₃, Z₄ must be a carbon atom to which the remainder of the molecule is attached.
259. Z₁, Z₃ are N; Z₂, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₂, Z₄ must be a carbon atom to which the remainder of the molecule is attached.
260. Z₁, Z₄ are N; Z₂, Z₃ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₂, Z₃ must be a carbon atom to which the remainder of the molecule is attached.
261. Z₂ is N; Z₁, Z₃, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₃, Z₄ must be a carbon atom to which the remainder of the molecule is attached.
262. Z₂, Z₃ are N; Z₁, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₄ must be a carbon atom to which the remainder of the molecule is attached.
263. Z₂, Z₄ are N; Z₁, Z₃ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₃ must be a carbon atom to which the remainder of the molecule is attached.
264. Z₂ is N; Z₁, Z₃, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₃, Z₄ must be a carbon atom to which the remainder of the molecule is attached.
265. Z₂, Z₃ are N; Z₁, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₄ must be a carbon atom to which the remainder of the molecule is attached.
266. Z₂, Z₄ are N; Z₁, Z₃ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₃ must be a carbon atom to which the remainder of the molecule is attached.
267. Z₂ is N; Z₁, Z₃, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₃, Z₄ must be a carbon atom to which the remainder of the molecule is attached.
268. Z₂, Z₃ are N; Z₁, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₄ must be a carbon atom to which the remainder of the molecule is attached.
269. Z₂, Z₄ are N; Z₁, Z₃ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₃ must be a carbon atom to which the remainder of the molecule is attached.
270. Z₃ is N; Z₁, Z₂, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₂, Z₄ must be a carbon atom to which the remainder of the molecule is attached.
271. Z₃, Z₄ are N; Z₁, Z₄ are independently CR₂; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₂ must be a carbon atom to which the remainder of the molecule is attached.
272. Z₃ is N; Z₁, Z₃, Z₄ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₂, Z₄ must be a carbon atom to which the remainder of the molecule is attached.
273. Z₃, Z₄ are N; Z₁, Z₂ are independently CR₂; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₂ must be a carbon atom to which the remainder of the molecule is attached.
274. Z₃ is N; Z₁, Z₃, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₂, Z₄ must be a carbon atom to which the remainder of the molecule is attached.
275. Z₃, Z₄ are N; Z₁, Z₄ are independently CR₂; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₂ must be a carbon atom to which the remainder of the molecule is attached.
276. Z₁, Z₂, Z₃ are independently CR₂; Z₄ is N; Z₅ is O, S, N—R₁; W₁, W₂, W₃ are independently CR₄R₄, S(O)r (r=0-2), O, N—R₁ with the proviso that no S—S, S—O or O—O bond formation can occur to form a saturated ring. In the ring formed by W₁, W₂, W₃ one double bond might be present; t=1-4; Y₁, Y₂, Y₃, Y₄ are C; Any one of Z₁, Z₂, Z₃, must be a carbon atom to which the remainder of the molecule is attached.
277. Z₁, Z₂, Z₃ are independently CR₂; Z₄ is N; Z₅ is N, CR₂; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-4; Y₁, Y₂, Y₃ are C; Y₄ is N; Any one of Z₁, Z₂, Z₃ must be a carbon atom to which the remainder of the molecule is attached.
278. Z₁, Z₂, Z₃ are independently CR₂; Z₄ is N; Z₅ is O, S. N; W₁, W₂, W₃ are independently CR₄R₄, S(O)r (r=0-2), O, N—R₁ with the proviso that no S—S, S—O or O—O bond formation can occur to form a saturated ring; In the ring formed by W₁, W₂, W₃ one double bond might be present; t=1-3; Y₁, Y₂, Y₃ are C; Y₄ is N; Any one of Z₁, Z₂, Z₃, Z₄ must be a carbon atom to which the remainder of the molecule is attached.

The preferred embodiments of formula 12-B:
279. Z₁, Z₂, Z₃, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄ must be a carbon atom to which the remainder of the molecule is attached.
280. Z₁ is N; Z₂, Z₃, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄ must be a carbon atom to which the remainder of the molecule is attached.
281. Z₁, Z₂ are N; Z₃, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₃, Z₄ must be a carbon atom to which the remainder of the molecule is attached.
282. Z₁, Z₃ are N; Z₂, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₄ must be a carbon atom to which the remainder of the molecule is attached.
283. Z₂ is N; Z₁, Z₃, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄ must be a carbon atom to which the remainder of the molecule is attached.
284. Z₂, Z₃ are N; Z₁, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₄ must be a carbon atom to which the remainder of the molecule is attached.
285. Z₃ is N; Z₁, Z₂, Z₄ are independently CR₂; Z₅ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄ must be a carbon atom to which the remainder of the molecule is attached.
286. Z₁, Z₂, Z₃, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅ must be a carbon atom to which the remainder of the molecule is attached.
287. Z₁ is N; Z₂, Z₃, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅ must be a carbon atom to which the remainder of the molecule is attached.
288. Z₁, Z₂ are N; Z₃, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₃, Z₅ must be a carbon atom to which the remainder of the molecule is attached.
289. Z₁, Z₃ are N; Z₂, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₅ must be a carbon atom to which the remainder of the molecule is attached.
290. Z₂ is N; Z₁, Z₃, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅ must be a carbon atom to which the remainder of the molecule is attached.
291. Z₂, Z₃ are N; Z₁, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₄, Z₅ must be a carbon atom to which the remainder of the molecule is attached.
292. Z₃ is N; Z₁, Z₂, Z₅ are independently CR₂; Z₄ is O, S, N—R₁; Y₁, Y₂, Y₃, Y₄ are C; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₅ must be a carbon atom to which the remainder of the molecule is attached.
293. Z₁, Z₂, Z₃, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄ must be a carbon atom to which the remainder of the molecule is attached.
294. Z₁ is N; Z₂, Z₃, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄ must be a carbon atom to which the remainder of the molecule is attached.
295. Z₁, Z₂ are N; Z₃, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₃, Z₄ must be a carbon atom to which the remainder of the molecule is attached.
296. Z₁, Z₃ are N; Z₂, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅ must be a carbon atom to which the remainder of the molecule is attached.
297. Z₂ is N; Z₁, Z₃, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄ must be a carbon atom to which the remainder of the molecule is attached.
298. Z₂, Z₃ are N; Z₁, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₄, Z₅ must be a carbon atom to which the remainder of the molecule is attached.
299. Z₃ is N; Z₁, Z₂, Z₄, Z₅ are independently CR₂; Y₁, Y₂, Y₃ are C; Y₄ is N; W₁, W₂ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅ must be a carbon atom to which the remainder of the molecule is attached.

The preferred embodiments of formula 13-A:
300. Z₁, Z₂, Z₃, Z₄, Z₅, Z₆ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅, Z₆ must be a carbon atom to which the remainder of the molecule is attached.
301. Z₁ is N; Z₂, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅, Z₆ must be a carbon atom to which the remainder of the molecule is attached.
302. Z₁, Z₂ are N; Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆ must be a carbon atom to which the remainder of the molecule is attached.
303. Z₁, Z₃ are N; Z₂, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅, Z₆ must be a carbon atom to which the remainder of the molecule is attached.
304. Z₁, Z₄ are N; Z₂, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅, Z₆ must be a carbon atom to which the remainder of the molecule is attached.
305. Z₄, Z₅ are N; Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅ must be a carbon atom to which the remainder of the molecule is attached.
306. Z₁, Z₆ are N; Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅ must be a carbon atom to which the remainder of the molecule is attached.
307. Z₂ is N; Z₁, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆ must be a carbon atom to which the remainder of the molecule is attached.
308. Z₂, Z₃ are N; Z₁, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₄, Z₅, Z₆ must be a carbon atom to which the remainder of the molecule is attached.
309. Z₂, Z₄ are N; Z₁, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅, Z₆ must be a carbon atom to which the remainder of the molecule is attached.
310. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
311. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
312. Z₂, Z₆ are N; Z₁, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅ must be a carbon atom to which the remainder of the molecule is attached.
313. Z₃ is N; Z₁, Z₂, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅, Z₆ must be a carbon atom to which the remainder of the molecule is attached.
314. Z₃, Z₄ are N; Z₁, Z₂, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₅, Z₆ must be a carbon atom to which the remainder of the molecule is attached.
315. Z₃, Z₅ are N; Z₁, Z₂, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₆ must be a carbon atom to which the remainder of the molecule is attached.
316. Z₃, Z₆ are N; Z₁, Z₂, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅ must be a carbon atom to which the remainder of the molecule is attached.
317. Z₄ is N; Z₁, Z₂, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
318. Z₄, Z₅ are N; Z₁, Z₂, Z₃, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
319. Z₅ is N; Z₁, Z₂, Z₃, Z₄, Z₆ are independently CR₂; Y₁, Y₂, Y₃, Y₄ are C; W₁, W₂ W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₆ must be a carbon atom to which the remainder of the molecule is attached.
320. Z₅, Z₆ are N; Z₁, Z₂, Z₃, Z₄ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄ must be a carbon atom to which the remainder of the molecule is attached.
321. Z₆ is N; Z₁, Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅ must be a carbon atom to which the remainder of the molecule is attached.

The preferred embodiments of formula 13-B:
322. Z₁, Z₂, Z₃, Z₄, Z₅, Z₆ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅, Z₆ must be a carbon atom to which the remainder of the molecule is attached.
323. Z₁ is N; Z₂, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅, Z₆ must be a carbon atom to which the remainder of the molecule is attached.
324. Z₁, Z₂ are N; Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆ must be a carbon atom to which the remainder of the molecule is attached.
325. Z₁, Z₃ are N; Z₂, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅, Z₆ must be a carbon atom to which the remainder of the molecule is attached.
326. Z₁, Z₄ are N; Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅, Z₆ must be a carbon atom to which the remainder of the molecule is attached.
327. Z₁, Z₅ are N; Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₆ must be a carbon atom to which the remainder of the molecule is attached.
328. Z₁, Z₆ are N; Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅ must be a carbon atom to which the remainder of the molecule is attached.
329. Z₂ is N; Z₁, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆ must be a carbon atom to which the remainder of the molecule is attached.
330. Z₂, Z₃ are N; Z₁, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₄, Z₅, Z₆ must be a carbon atom to which the remainder of the molecule is attached.
331. Z₂, Z₄ are N; Z₁, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅, Z₆ must be a carbon atom to which the remainder of the molecule is attached.
332. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
333. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
334. Z₂, Z₆ are N; Z₁, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅ must be a carbon atom to which the remainder of the molecule is attached.
335. Z₃ is N; Z₁, Z₂, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅, Z₆ must be a carbon atom to which the remainder of the molecule is attached.
336. Z₃, Z₄ are N; Z₁, Z₂, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₅, Z₆ must be a carbon atom to which the remainder of the molecule is attached.
337. Z₃, Z₅ are N; Z₁, Z₂, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₆ must be a carbon atom to which the remainder of the molecule is attached.
338. Z₃, Z₆ are N; Z₁, Z₂, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅ must be a carbon atom to which the remainder of the molecule is attached.
339. Z₄ is N; Z₁, Z₂, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
340. Z₄, Z₅ are N; Z₁, Z₂, Z₃, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
341. Z₅ is N; Z₁, Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₆ must be a carbon atom to which the remainder of the molecule is attached.
342. Z₅, Z₆ are N; Z₁, Z₂, Z₃, Z₄ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄ must be a carbon atom to which the remainder of the molecule is attached.
343. Z₆ is N; Z₁, Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅ must be a carbon atom to which the remainder of the molecule is attached.

The preferred embodiments of formula 13-C:
344. Z₁, Z₂, Z₃, Z₄, Z₅, Z₆ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅, Z₆ must be a carbon atom to which the remainder of the molecule is attached.
345. Z₁ is N; Z₂, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅, Z₆ must be a carbon atom to which the remainder of the molecule is attached.
346. Z₁, Z₂ are N; Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆ must be a carbon atom to which the remainder of the molecule is attached.
347. Z₁, Z₃ are N; Z₂, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅, Z₆ must be a carbon atom to which the remainder of the molecule is attached.
348. Z₁, Z₄ are N; Z₂, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅, Z₆ must be a carbon atom to which the remainder of the molecule is attached.
349. Z₁, Z₅ are N; Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₆ must be a carbon atom to which the remainder of the molecule is attached.
350. Z₁, Z₆ are N; Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅ must be a carbon atom to which the remainder of the molecule is attached.
351. Z₂ is N; Z₁, Z₃, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆ must be a carbon atom to which the remainder of the molecule is attached.
352. Z₂, Z₃ are N; Z₁, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₄, Z₅, Z₆ must be a carbon atom to which the remainder of the molecule is attached.
353. Z₂, Z₄ are N; Z₁, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅, Z₆ must be a carbon atom to which the remainder of the molecule is attached.
354. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
355. Z₂, Z₅ are N; Z₁, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₆ must be a carbon atom to which the remainder of the molecule is attached.
356. Z₂, Z₆ are N; Z₁, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅ must be a carbon atom to which the remainder of the molecule is attached.
357. Z₃ is N; Z₁, Z₂, Z₄, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅, Z₆ must be a carbon atom to which the remainder of the molecule is attached.
358. Z₄ are N; Z₁, Z₂, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₅, Z₆ must be a carbon atom to which the remainder of the molecule is attached.
359. Z₃, Z₅ are N; Z₁, Z₂, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₆ must be a carbon atom to which the remainder of the molecule is attached.
360. Z₃, Z₆ are N; Z₁, Z₂, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅ must be a carbon atom to which the remainder of the molecule is attached.
361. Z₄ is N; Z₁, Z₂, Z₃, Z₅, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
362. Z₄, Z₅ are N; Z₁, Z₂, Z₃, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₅, Z₆ must be a carbon atom to which the remainder of the molecule is attached.
363. Z₅ is N; Z₁, Z₂, Z₃, Z₄, Z₆ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₆ must be a carbon atom to which the remainder of the molecule is attached.
364. Z₅, Z₆ are N; Z₁, Z₂, Z₃, are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄ must be a carbon atom to which the remainder of the molecule is attached.
365. Z₆ is N; Z₁, Z₂, Z₃, Z₄, Z₅ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₃, Z₄, Z₅ must be a carbon atom to which the remainder of the molecule is attached.

The preferred embodiments of formula 14-A and 14-B:
366. Z₁ is N; Z₂, Z₃, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅, Z₆, Z₇, Z₈ must be a carbon atom to which the remainder of the molecule is attached.
367. Z₁, Z₂ are N; Z₃, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆, Z₇, Z₈ must be a carbon atom to which the remainder of the molecule is attached.
368. Z₁, Z₃ are N; Z₂, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅, Z₆, Z₇, Z₈ must be a carbon atom to which the remainder of the molecule is attached.
369. Z₁, Z₄ are N; Z₂, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅, Z₆, Z₇, Z₈ must be a carbon atom to which the remainder of the molecule is attached.
370. Z₁, Z₂ are N; Z₃, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆, Z₇, Z₈ must be a carbon atom to which the remainder of the molecule is attached.
371. Z₁ is N; Z₂, Z₃, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₄, Z₅, Z₆, Z₇, Z₈ must be a carbon atom to which the remainder of the molecule is attached.
372. Z₁, Z₂ are N; Z₃, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆, Z₇, Z₈ must be a carbon atom to which the remainder of the molecule is attached.
373. Z₁, Z₃ are N; Z₂, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₄, Z₅, Z₆, Z₇, Z₈ must be a carbon atom to which the remainder of the molecule is attached.
374. Z₁, Z₄ are N; Z₂, Z₃ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₂, Z₃, Z₅, Z₆, Z₇, Z₈ must be a carbon atom to which the remainder of the molecule is attached.
375. Z₁, Z₂ are N; Z₃, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₃, Z₄, Z₅, Z₆, Z₇, Z₈ must be a carbon atom to which the remainder of the molecule is attached.
376. Z₂ is N; Z₁, Z₃, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆, Z₇, Z₈ must be a carbon atom to which the remainder of the molecule is attached.
377. Z₂, Z₃ are N; Z₁, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₄, Z₅, Z₆, Z₇, Z₈ must be a carbon atom to which the remainder of the molecule is attached.
378. Z₂, Z₄ are N; Z₁, Z₃, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅, Z₆, Z₇, Z₈ must be a carbon atom to which the remainder of the molecule is attached.
379. Z₂ is N; Z₁, Z₃, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆, Z₇, Z₈ must be a carbon atom to which the remainder of the molecule is attached.
380. Z₂, Z₃ are N; Z₁, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₄, Z₅, Z₆, Z₇, Z₈ must be a carbon atom to which the remainder of the molecule is attached.
381. Z₂, Z₄ are N; Z₁, Z₃ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₅, Z₆, Z₇, Z₈ must be a carbon atom to which the remainder of the molecule is attached.
382. Z₃ is N; Z₁, Z₂, Z₄, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₄, Z₅, Z₆, Z₇, Z₈ must be a carbon atom to which the remainder of the molecule is attached.
383. Z₃, Z₄ are N; Z₁, Z₂, Z₅, Z₆, Z₇, Z₈ are independently CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₂, Z₅, Z₆, Z₇, Z₈ must be a carbon atom to which the remainder of the molecule is attached.
384. Z₄ is N; Z₁, Z₃, Z₄ are independently CR₂; Z₅, Z₆, Z₇, Z₈ are independently N, CR₂; Y₁, Y₂, Y₃, Y₄ are C; W₁, W₂, W₃ are independently CR₄R₄, S(O)r (r=0-2), O, N—R₁ with the proviso that no S—S, S—O or O—O bond formation can occur to form a saturated ring; t=1-3. Any one of Z₁, Z₃, Z₄, Z₅, Z₆, Z₇, Z₈ must be a carbon atom to which the remainder of the molecule is attached.

The preferred embodiments of formula 15-A:
385. Z₁ is N; Z₂, Z₃, Z₄ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₂, Z₃, Z₄ must be a carbon atom to which the remainder of the molecule is attached.
386. Z₁, Z₂ are N; Z₃, Z₄ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₃, Z₄ must be a carbon atom to which the remainder of the molecule is attached.
387. Z₁, Z₃ are N; Z₂, Z₄ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₂, Z₄ must be a carbon atom to which the remainder of the molecule is attached.
388. Z₁, Z₄ are N; Z₂, Z₃ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₂, Z₃ must be a carbon atom to which the remainder of the molecule is attached.
389. Z₂ is N; Z₁, Z₃, Z₄ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁; t=0-2; W₃, W₄, W₅ are independently O, S(O)r (r=0-2), CR₄R₄, N—R₁ with the proviso that no S—S, S—O or O—O bond formation can occur to form a saturated ring; u=1-3; Any one of Z₁, Z₃, Z₄ must be a carbon atom to which the remainder of the molecule is attached.
390. Z₂, Z₃ are N; Z₁, Z₄ are independently CR₂; Y₁, Y₂ are C; Y₃, Y₄ are independently CH, N; Wt is O, S(O)r (r=0-2), CR₄R₄, N—R₁, a bond; W₂ is O, S(O)r (r=0-2), CR₄R₄, N—R₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₁, Z₄ must be a carbon atom to which the remainder of the molecule is attached.
391. Z₂, Z₄ are N; Z₁, Z₃ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₁, Z₃ must be a carbon atom to which the remainder of the molecule is attached.
392. Z₃ is N; Z₁, Z₂, Z₄ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁; t=0-2; W₃, W₄, W₅ are independently O, S(O)r (r=0-2), CR₄R₄, N—R₁ with the proviso that no S—S, S—O or O—O bond formation can occur to form a saturated ring; u=1-3; Any one of Z₁, Z₂, Z₄ must be a carbon atom to which the remainder of the molecule is attached.
393. Z₃, Z₄ are N; Z₁, Z₂ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₁, Z₂ must be a carbon atom to which the remainder of the molecule is attached.
394. Z₄ is N; Z₁, Z₂, Z₃ are independently CR₂; Y₁, Y₂ are C; Y₃, Y₄ are independently CH, N; W₁ is O, S(O)r (r=0-2), CR₄R₄, N—R₁, a bond; W₂ is O, S(O)r (r=0-2), CR₄R₄, N—R₁; t=0-2; W₃, W₄, W₅ are independently O, S(O)ᵣ (r=0-2), CR₄R₄, N—R₁ with the proviso that no S—S, S—O or O—O bond formation can occur to form a saturated ring; u=1-3; Any one of Z₁, Z₂, Z₃ must be a carbon atom to which the remainder of the molecule is attached.

The preferred embodiments of formula 15-B
395. Z₁ is N; Z₂, Z₃ Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₂, Z₃, Z₄ must be a carbon atom to which the remainder of the molecule is attached.
396. Z₁, Z₂ are N; Z₃, Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₃, Z₄ must be a carbon atom to which the remainder of the molecule is attached.
397. Z₁, Z₃ are N; Z₂, Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₂, Z₄ must be a carbon atom to which the remainder of the molecule is attached.
398. Z₁, Z₄ are N; Z₂, Z₃ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₂, Z₃ must be a carbon atom to which the remainder of the molecule is attached.
399. Z₂ is N; Z₁, Z₃, Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₁, Z₃, Z₄ must be a carbon atom to which the remainder of the molecule is attached.
400. Z₂, Z₃ are N; Z₁, Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₁, Z₄ must be a carbon atom to which the remainder of the molecule is attached.
401. Z₂, Z₄ are N; Z₁, Z₃ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₁, Z₃ must be a carbon atom to which the remainder of the molecule is attached.
402. Z₃ is N; Z₁, Z₂, Z₄ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one Z₁, Z₂, Z₄ of must be a carbon atom to which the remainder of the molecule is attached.
403. Z₃, Z₄ are N; Z₁, Z₂ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one of Z₁, Z₂ must be a carbon atom to which the remainder of the molecule is attached.
404. Z₄ is N; Z₁, Z₂, Z₃ are independently CR₂; Y₁, Y₂ are C; Y₃, Y₄ are independently CH, N; W₁ is O, S(O)r (r=0-2), CR₄R₄, N—R₁; W₂, W₃, W₄ are independently O, S(O)r (r=0-2), CR₄R₄, N—R₁ with the proviso that no S—S, S—O or O—O bond formation can occur to form a saturated ring; t=1-2; u=1-3; Any one Z₁, Z₂, Z₃ of must be a carbon atom to which the remainder of the molecule is attached.

The preferred embodiments of formula 15-C:
405. Z₁, Z₂, Z₃ are independently CR₂; Y₁, Y₂, Y₄ are C; Y₂ is N; W₁, W₂, W₄ are independently O, S(O)r (r=0-2), CR₄R₄, N—R₁ with the proviso that no S—S, S—O or O—O bond formation can occur to form a saturated ring; t=1-2; u=1-2; Any one Z₁, Z₂, Z₃ of must be a carbon atom to which the remainder of the molecule is attached.
406. Z₁ is N; Z₂, Z₃ are independently CR₂; Y₁, Y₂, Y₄ are C; Y₂ is N; W₁, W₂, W₄ are independently O, S(O)r (r=0-2), CR₄R₄, N—R₁ with the proviso that no S—S, S—O or O—O bond formation can occur to form a saturated ring; t=1-2; u=1-2; Any one Z₂, Z₃ of must be a carbon atom to which the remainder of the molecule is attached.
407. Z₁, Z₃ are N; Z₃ is CR₂; Y₁, Y₂, Y₄ are C; Y₂ is N; W₁, W₂, W₄ are independently O, S(O)r (r=0-2), CR₄R₄, N—R₁ with the proviso that no S—S, S—O or O—O bond formation can occur to form a saturated ring; t=1-2; u=1-2; Z₃ is the carbon atom to which the remainder of the molecule is attached.
   The expression ‘fused bicyclic heteroaryl group’ is used in the specification and claims to mean:

A group comprising two fused rings in which one has aromatic character [i.e. Huckel's rule (4n+2)] and the other ring is non-aromatic;

The fused bicyclic heteroaryl group contains one to six heteroatoms selected from the group O, S, N and N—R₁;

The fused bicyclic heteroaryl group is bonded to the remainder of the molecule through a carbon atom in the aromatic ring as shown in the formula I;

The aromatic ring of the fused bicyclic heteroaryl group contains five or six ring atoms (including bridgehead atoms) selected from CR₂, N, O, S or N—R₁. The aromatic ring of the fused bicyclic heteroaryl group contains 0 to 3 heteroatoms selected from the group O, S, N and N—R₁;

The non-aromatic ring of the fused bicyclic heteroaryl group contains five to eight ring atoms (including bridgehead atoms) selected from CR₄R₄, N, N—R₁, O, S(O)ₙ where n=0-2. The non-aromatic ring of the fused bicyclic heteroaryl group contains 0 to 4 heteroatoms selected from N, N—R₁, O or S(O)ₙ where n=0 to 2.

Examples of fused bicyclic heteroaryl groups are optionally substituted ring systems such as one of the following:
4,5,6,7-tetrahydrothieno[3,2-c]pyridine, optionally substituted by e.g., arylalkyl such as benzyl; by alkoxyarylalkyl such as 4-methoxybenzyl; by C1-C6alkyl such as methyl; by heteroarylalkyl such as pyridin-3-ylmethyl; by arylalkylCO— such as phenylacetyl; or heteroarylCO— such as pyridin-3-ylcarbonyl; e.g. by alkylCO-such as acetyl;
5,6,7,8-tetrahydroimidazo[1,2-a]pyrazine, optionally substituted e.g., by C1-C6alkyl such as methyl;
5,6-dihydro-8H-imidazo[2,1-c][1,4]thiazine;
6,7-Dihydro-5H-pyrrolo[1,2-a]imidazole
5,6-Dihydro-8H-imidazo[2,1-c][1,4]oxazine
5,6-Dihydro-4H-pyrrolo[1,2-b]pyrazole
4,5,6,7-tetrahydropyrazolo[1,5-a]pyridine
6-oxo-5,6,7,8-tetrahydro-imidazo[1,2-a]pyrazine, optionally substituted e.g., by C1-C6alkyl such as methyl;
6,7-Dihydro-4H-pyrazolo[5,1-c][1,4]thiazine;
4H-5-thia-1,6a-diazapentalene;
7H-Imidazo[1,2-c]thiazole;
4-oxo-6,7-dihydro-4H-pyrazolo[5,1-c][1,4]oxazine;
6,7-Dihydro-4H-thieno[3,2-c]pyran;
6,7-Dihydro-4H-thieno[3,2-c]thiopyran;
6,7-dihydro-4H-thieno[3,2-c]pyridine, optionally substituted by C2-C7alkoxycarbonyl;
6,7,8,9-tetrahydro-5H-imidazo[1,2-a]azepine;
5,6,7,8-tetrahydroimidazo[1,2-a]pyrazine, optionally substituted by arylalkyl such as benzyl;
5,5-Dioxo-4,5,6,7-tetrahydro-5λ⁶-pyrazolo[5,1-c][1,4]thiazine;
4,5,6,7-tetrahydropyrazolo[1,5-a]pyrazine;
5,6-Dihydro-4H-cyclopenta[b]furan;
4,5-Dihydro-6-thia-1,7a-diazaindene;
5,6-Dihydro-8-H-imidazo[2,1-c][1,4]thiazine;
4H-5-thia-1,6a-diazapentalene;
2,3-Dihydropyrazolo[5,1-b]thiazole;
2,3-Dihydropyrazolo[5,1-b]oxazole;
6,7-dihydro-4H-pyrazolo[5,1-c][1,4]oxazine;
6,7-5H-Dihydropyrazolo[5,1-b]oxazine; and
4,5,6,7-tetrahydropyrazolo[1,5-a]pyrazine optionally substituted, e.g, by alkoxyalkylCO— such as 2-methoxyacetyl; or by alkyloxyalkylCO— such as methoxyacetyl.

Further examples of bicyclic heteroarylgroups are as follows:

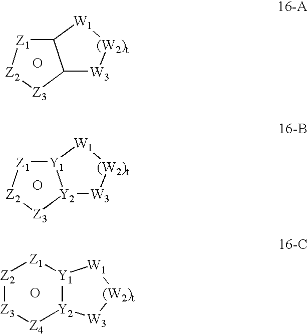

In formula 16-A Z₁, Z₂ and Z₃ are independently CR₂, N, O, S or

N—R₁ and one of Z₁-Z₃ is carbon and is bonded to the remainder of the molecule as shown in formula I. When one of Z's is CR₂ the other two Zs can be either two N or one N and O, S, N—R₁, in any combinations with out disrupting the aromaticity; when two Z,s=CR₂ the other Z can be optionally selected from one N, O, S or N—R₁, in any combination with out disrupting the aromaticity;

W₁, W₂ and W₃ are independently CR₄R₄, S, SO, SO₂, O, N—R₁, C═O; with the proviso that no S—S or O—O or S—O bond formation can occur to form the saturated ring system; t=1 to 4.

In formula 16-B Z1, Z2 and Z3 are independently CR₂, N, O, S or N—R₁ and one of Z1-Z3 is carbon and is bonded to the remainder of the molecule as shown in formula I.

When one of Z's=CR₂, then the other two Z's can be independently CR₂, N, O, S or N—R₁ in any combinations with out disrupting the aromaticity;

When two Z's=N, then the other carbon in the ring is bonded to the penem portion of the molecule as shown in formula I.

W₁, W₂ and W₃ are independently CR₄R₄, S, SO, SO₂, O, N—R₁,

t=1 to 4;

Y₁ and Y₂=N or C; with the proviso that when the aromatic heterocycle is imidazole, the saturated ring may not contain a S adjacent to the bridgehead carbon.

In formula 16-C Z1, Z2, Z3 and Z4 are independently CR₂ or N and one of Z1-Z4 is carbon and is bonded to the remainder of the molecule. W₁, W₂ and W₃ are independently CR₄R₄, S, SO, SO₂, O, or N—R₁; with the proviso that no S—S or O—O or S—O bond formation can occur to form the saturated ring system; t=1 to 4.

Y₁ and Y₂ are independently C or N.

The more preferred embodiment of the formula 16-A:
1. t=1 to 3.
2. In formula 16-A Z1 is N, S, N—R₁ or O and one of Z2 or Z3 is CR₂ and the other of Z2 or Z3 is carbon and is bonded to the remainder of the molecule as shown in formula I.
3. In formula 16-A Z3 is N, S, N—R₁ or O and one of Z2 or Z1 is CR₂ and the other of Z2 or Z1 is carbon and is bonded to the remainder of the molecule as shown in formula I.
4. In formula 16-A Z2 is N, S, N—R₁ or O and one of Z1 or Z3 is CR₂ and the other of Z1 or Z3 is carbon bonded to the remainder of the molecule as shown in formula I.
5. In formula 16-A Z1 is N, N—R₁, O or S and Z2 is N, O or S and Z3 is a carbon bonded to the penem portion of the molecule as shown in formula I.
6. In formula 16-A Z3 is N, N—R₁, O or S and Z2 is N, O or S and Z1 is a carbon bonded to the penem portion of the molecule as shown in formula I.
7. In formula 16-A Z1 is N, N—R₁, O or S and Z3 is N, O or S and Z2 is a carbon bonded to the penem portion of the molecule as shown in formula I.
8. In formula 16-A Z1 is N, S, N—R₁ or O and Z2 or Z3 is CR₂ and the other of Z2 or Z3 is carbon and is bonded to the remainder of the molecule; W₁, W₂ and W₃ are independently CR4R4.
9. In formula 16-A Z3 is N, S, N—R₁ or O and one of Z2 or Z1 is CR₂ and the other of Z2 or Z1 is carbon and is bonded to the remainder of the molecule; W₁, W₂, and W₃ are independently CR4R4.
10. In formula 16-A Z2 is N, S, N—R₁ or O and one of Z1 or Z3 is CR₂ and the other of Z1 or Z3 is carbon and is bonded to the remainder of the molecule; W₁, W₂, and W₃ are independently CR4R4.
11. In formula 16-A Z1 is N, N—R₁, O or S and Z2 is N, O or S; Z3 is a carbon bonded to the penem portion of the molecule; W₁, W₂, W₃ are independently CR4R4.
12. In formula 16-A Z3 is N, N—R₁, O or S; Z2 is N, O or S; Z1 is a carbon bonded to the penem portion of the molecule; W₁, W₂, W₃ are independently CR4R4
13. In formula 16-A Z1 is N, N—R₁, O or S; Z3 is N, O or S; Z2 is a carbon bonded to the penem portion of the molecule; W₁, W₂, W₃ are independently CR4R4.
14. In formula 16-A Z3 is N, N—R₁, O or S; Z1 is N, O or S; Z2 is a carbon bonded to the penem portion of the molecule; W₁, W₂, W₃ are independently CR4R4.
15. In formula 16-A Z1 is N, S, N—R₁ or O; one of Z2 or Z3 is CR₂ and the other of Z2 or Z3 is carbon and is bonded to the remainder of the molecule, t=1-3; one W₂ is N—R₁, O or S(O)ₙ n=0-2 and another W₂ is CR4R4.
16. In formula 16-A Z3 is N, S, N—R₁ or O; one of Z2 or Z1 is CR₂ and the other of Z2 or Z1 is carbon and is bonded to the remainder of the molecule, t=1-3; one W₂ is N—R₁, O or S(O), n=0-2 and another W₂=CR4R4.
17. In formula 16-A Z2 is N, S, N—R₁ or O; one of Z1 or Z3 is CR₂ and the other of Z1 or Z3 is carbon and is bonded to the remainder of the molecule; t=1-3; one W₂ is N—R₁, O or S(O)ₙ n=0-2 and another W₂ is CR4R4.
18. In formula 16-A when Z1=N, N—R₁, O or S and Z2=N, O or S and Z3=a carbon bonded to the penem portion of the molecule where t=1-3 then one W₂=N—R₁, O or S(O)ₙ n=0-2 and other W₂=CR4R4.
19. In formula 16-A Z3=N, N—R₁, O or S and Z2=N, O or S and Z1=a carbon bonded to the penem portion of the molecule where t=1-3 then one W₂=N—R₁, O or S(O)ₙ n=0-2 and other W₂=CR4R4.
20. In formula 16-A when Z1=N, N—R₁, O or S and Z3=N, O or S and Z2=a carbon bonded to the penem portion of the molecule where t=1-3 then one W₂=N—R₁, O or S(O)ₙ n=0-2 and other W₂=CR4R4.
21. In formula 16-A Z1=N, S, N—R₁ or O and Z2 or Z3=CR₂ and the other of Z2 or Z3 is carbon and is bonded to the remainder of the molecule; then W₁ and W₃=CH₂ or both hydrogens on the methylene linkage can be substituted to form a spiro system with or without the presence of hetero atoms selected from O, S═(O)ₙ (n=0 to 2), N—R₁ to form five to eight membered cyclic system; t=1-3; one W₂=N—R₁, O or S(O)ₙ n=0-2 and other W₂=CR4R4.
22. In formula 16-A Z3=N, S, N—R₁ or O and Z2 or Z1=CR₂ and the other of Z2 or Z1 is carbon and is bonded to the remainder of the molecule; then W₁ and W₃=CR4R4; where t=1-3 then one W₂=N—R₁, O or S(O)ₙ n=0-2 and other W₂=CR4R4.
23. In formula 16-A Z2=N, S, N—R₁ or O and Z1 or Z3=CR₂ and the other of Z1 or Z3 is carbon and is bonded to the remainder of the molecule; then W₁ and W₃=CR4R4, where t=1-3 then one W₂=N—R₁, O or S(O)ₙ n=0-2 and other W₂=CR4R4.
24. In formula 16-A when Z1=N, N—R₁, O or S and Z2=N, O or S then Z3=a carbon bonded to the penem portion of the molecule; then W₁ and W₃=CR4R4, where t=1-3 then one W₂=N—R₁, O or S(O)ₙ n=0-2 and other W₂=CR4R4.
25. In formula 16-A Z3=N, N—R₁, O or S and Z2=N, O or S then Z1=a carbon bonded to the penem portion of the molecule; then W₁ and W₃=CR4R4, where t=1-3 then one W₂=N—R₁, O or S(O)ₙ n=0-2 and other W₂=CR4R4.
26. In formula 16-A when Z1=N, N—R₁, O or S and Z3=N, O or S then 72=a carbon bonded to the penem portion of the molecule; then W₁ and W₃=CR4R4; t=1-3; one W₂ is N—R₁, O or S(O)ₙ n=0-2 and another W₂ is CR4R4.
27. In formula 16-A Z3 is N, N—R₁, O or S; Z1 is N, O or S; Z2 is a carbon bonded to the remainder of the molecule; W₁ and W₃ are independently CR4R4; t=1-3; one W₂ is N—R₁, O or S(O)ₙ n=0-2 and another W₂ is CR4R4.

The more preferred embodiments of the formula 16-B:
28. In formula 16-B t=3.
29. In formula 16-B Z1 and Z3 are N; Y₁ is N; Y2 is C and Z2 is carbon and is bonded to the remainder of the molecule as shown in formula I.
30. In formula 16-B Z2 and Z3 are N; Y1 is N; Y2 is C and Z1 is carbon and is bonded to the remainder of the molecule as shown in formula I.
31. In formula 16-B Z1 is N, Y1 is N, Y2 is C, one of Z₂ or Z₃ is CR₂ and the other of Z2 or Z3 is a carbon and is bonded to the remainder of the molecule as shown in formula I.
32. In formula 16-B Z1 is N, Y1 is C, Y2 is N, one of Z₂ or Z₃ is CR₂ and the other of Z2 or Z3 is a carbon and is bonded to the remainder of the molecule as shown in formula I.
33. In formula 16-B Z1 is N, Y1 is N, Y2 is C, one of Z₂ or Z₃ is CR₂ and the other of Z2 or Z3 is a carbon and is bonded to the remainder of the molecule as shown in formula I; W₁ and W₃ are independently CR4R4; t=1-3; one W₂ is N—R₁, O, S═(O)ₙ (n=0-2), and another W₂ is CR4R4.
34. In formula 16-B Z1 is N, Y1 is C, Y2 is N, one of Z2 or Z3 is CR₂ and the other of Z2 or Z3 is a carbon and is bonded to the remainder of the molecule as shown in formula I; W₁ and W₃ are independently CR4R4; t=1-3; one W₂ is N—R₁, O, S═(O)ₙ (n=0-2), and another W₂ is CR4R4.
35. In formula 16-B Z3 is N; Y1 is N; Y2 is C; one of Z1 or 79 is CR₂ and the other of Z₁ or Z₂ is carbon and is bonded to the remainder of the molecule as shown in formula I.
36. In formula 16-B Z2 is N; Y1 is N; Y2 is C; one of Z1 or Z3 is CR₂ and the other of Z₁ or Z₃ is carbon and is bonded to the remainder of the molecule as shown in formula I.
37. In formula 16-B Z1 and Z2 are N; Y1 is N; Y2 is C; and Z3 is carbon and is bonded to the remainder of the molecule as shown in formula I.
38. In formula 16-B Z1, Z2 and Z3 are independently CR₂; Y1 is C; Y2 is N; except one of Z1-Z3 is carbon and is bonded to the remainder of the molecule as shown in formula I.
39. In formula 16-B Z1 and Z3 are N; Y1 is N; Y2 is C; Z2 is carbon and is bonded to the remainder of the molecule as shown in formula I; and t=1-3.
40. In formula 16-B Z2 and Z3 are N; Y1 is N; Y2 is C; and Z1 is carbon and is bonded to the remainder of the molecule; and t=1-3;
41. In formula 16-B Z2 and Z3 are N, Y1 is C and Y2=N and Z1 is carbon and is bonded to the remainder of the molecule and t=1-3;
42. In formula 16-B Z2 and Z3 are N, Y1 is N; Y2 is C; Z1 is carbon and is bonded to the remainder of the molecule; W₁ and W₃ are independently CH₂ or both hydrogens on the methylene linkage can be substituted to form a spiro system with or without the presence of hetero atoms selected from O, S(O)ₙ n=0-2, N—R₁ to form five to eight membered cyclic system; t=1-3 and W₂ is CH₂, N—R₁, O, S(O)ₙ where n=0-2.
43. In formula 16-B Z3 is N; Y1 is N; Y2 is C; Z1 is CR₂ and Z2 is the carbon atom bonded to the remainder of the molecule.
44. In formula 16-B Z3 is N; Y1 is N; Y2 is C; Z1 is CR₂; Z2 is the carbon atom bonded to the remainder of the molecule; W₁, W₂ and W₃ are independently CR4R4; t=1 to 3.
45. In formula 16-B Z3 is N; Y1 is N; Y2 is C; Z1 is CR₂; Z2 is the carbon atom bonded to the remainder of the molecule; W₁ and W₃ are independently CR4R4; and one of W₂ is N—R₁, O or S(O)ₙ, and another W₂ is CR4R4; t=1-3.
46. In formula 16-B Z3 is N; Y1 is N; Y2 is C; Z1 is CR₂; Z2 is the carbon atom bonded to the remainder of the molecule; W₁ and W₂ are independently CR4R4; W₃ is N—R₁, O or S(O)ₙ; and t=2.
47. In formula 16-B Z3 is N; Y1 is N; Y2 is C; Z1 is CR₂; Z2 is the carbon atom bonded to the remainder of the molecule; W₁ and W₃ are independently CR4R4; W₂ is N—R₁, O or S(O)ₙ; and t=1.
48. In formula 16-B Z2 is N; Y1 is N; Y2 is C; Z3 is CR₂; Z1 is the carbon bonded to the remainder of the molecule; W₁ and W₂ is CH₂ or both hydrogens on the methylene linkage can be substituted to form a spiro system with or without the presence of hetero atoms selected from O, S(O)ₙ n=0-2, N—R₁ to form five to eight membered cyclic system; W₃ is N—R₁, O or S(O)ₙ; and t=3.
49. In formula 16-B Z2 is N; Y1 is N; Y2 is C, Z3 is CR₂; Z1 is the carbon bonded to the remainder of the molecule; W₁ and W₃ are independently CH₂ or both hydrogens on the methylene linkage can be substituted to form a spiro system with or without the presence of hetero atoms selected from O, S(O)ₙ n=0-2, N—R₁ to form five to eight membered cyclic system; and one W₂ is N—R₁, O or S(O)ₙ and another W₂ is CR4R4; and t=2.
50. In formula 16-B Z2 is N; Y1 is N; Y2 is C; Z3 is CR₂; Z1 is the carbon bonded to the remainder of the molecule; W₁ and W₃ are independently CH₂ or both hydrogens on the methylene linkage can be substituted to form a spiro system with or without the presence of hetero atoms selected from O, S(O)ₙ n=0-2, N—R₁ to form five to eight membered cyclic system; W₂ is N—R₁, O or S(O)ₙ; and t=1.
51. In formula 16-B Z2 is N; Y1 is N; Y2 is C; Z1 is CR₂; Z3 is the carbon bonded to the remainder of the molecule; W₁ and W₃ are independently CR4R4; one of W₂ is N—R₁, O or S(O)ₙ and another W₂ is CR4R4; and t=3.
52. In formula 16-B Z2 is N; Y1 is N; Y2 is C; Z1 is CR₂; Z3 is the carbon bonded to the remainder of the molecule; W₁ and W₃ are independently CR4R4; one W₂ is N—R₁, O or S(O)ₙ, and another W₂ is CR4R4; and t=2.
53. In formula 16-B Z2 is N; Y1 is N; Y2 is C; Z1 is CR₂; Z3 is the carbon bonded to the remainder of the molecule; W₁ and W₃ are independently CR4R4; W₂ is N—R₁, O or S(O)ₙ; and t=1.
54. In formula 16-B Z1 and Z2 are N; Y1 is N; Y2 is C; Z3 is carbon and is bonded to the remainder of the molecule; W₁ and W₃ are independently CR4R4; one of W₂ is N—R₁, O or S(O)ₙ and another W₂ is CR4R4; and t=3.
55. In formula 16-B Z1 and Z2 are N; Y1 is N; Y2 is C; Z3 is carbon and is bonded to the remainder of the molecule; W₁ and W₃ are independently CR4R4; one of W₂ is N—R₁, O or S(O)ₙ and another W₂ is CR4R4; and t=2.
56. In formula 16-B Z1 and Z2 are N; Y1 is N; Y2 is C; Z3 is carbon and is bonded to the remainder of the molecule; W₁ and W₃ are independently CR4R4; W₂ is N—R₁, O or S(O)ₙ; and t=1
57. In formula 16-B Z1 and Z2 are independently CR₂; Y1 is C; Y2 is N; Z3 is carbon and is bonded to the remainder of the molecule; W₁ and W₃ are independently CR4R4; one of W₂ is N—R₁, O or S(O)ₙ; another W₂ is CR4R4; and t=3.
58. In formula 16-B Z1 and Z2 are independently CR₂; Y1 is C and Y2 is N and Z3 is carbon and is bonded to the remainder of the molecule; W₁ and W₃ are independently CR4R4; and one W₂ is N—R₁, O or S(O)ₙ and the other W₂ is CR4R4; and t=2.
59. In formula 16-B Z1 and Z2 are independently CR₂; Y1 is C; Y2 is N; Z3 is carbon and is bonded to the remainder of the molecule; W₁ and W₃ are independently CR4R4; W₂ is N—R₁, O or S(O)ₙ; and t=1.
60. In formula 16-B Z1 and Z3 are independently CR₂; Y1 is C; Y2 is N; Z2 is carbon and is bonded to the remainder of the molecule; W₁ and W₃ are independently CR4R4; one W₂ is N—R₁, O or S(O)ₙ; another W₂ is CR4R4; and t=3.
61. In formula 16-B Z1 and Z3 are independently CR₂; Y1 is C; Y2 is N; Z2 is carbon and is bonded to the remainder of the molecule; W₁ and W₃ are independently CR4R4; and one W₂ is N—R₁, O or S(O)ₙ and the other W₂ is CR4R4; and t=2.
62. In formula 16-B Z1 and Z3 are independently CR₂; Y1 is C; Y2 is N; Z2 is carbon and is bonded to the remainder of the molecule; W₁ and W₃ are independently CR4R4; W₂ is N—R₁, O or S(O)ₙ; and t=1.
63. In formula 16-B Z3 and Z2 are independently CR₂; Y1 is C; Y2 is N; Z1 is carbon and is bonded to the remainder of the molecule; W₁ and W₂ are independently CR4R4; one W₂ is N—R₁, O or S(O)ₙ; another W₂ is CR4R4; and t=3.
64. In formula 16-B Z3 and Z2 are independently CR₂; Y1 is C; Y2 is N; Z1 is carbon and is bonded to the remainder of the molecule; W₁ and W₃ are independently CR4R4; one W₂ is N—R₁, O or S(O)ₙ; the other W₂ is CR4R4; and t=2.
65. In formula 16-B Z3 and Z2 are independently CR₂; Y1 is C; Y2 is N; Z1 is carbon and is bonded to the remainder of the molecule; W₁ and W₃ are independently CR4R4; W₂ is N—R₁, O or S(O)ₙ; and t=1.
66. In formula 16-B Z3 is N; Y1 is N; Y2 is C; one of Z1 and Z2 is CR₂ and the other is C; W₁ is CR4R4; W₂ is CR4R4; W₃ is CH₂, N—R₁ or O; and t=1.
67. In formula 16-B Z3 is N; Y1 is N; Y2 is C; one of Z1 and Z2 is CR₂ and the other is C; W₁ is CR4R4; W₂ is C═O; W₃ is N—R₁; and t=1.
68. In formula 16-B Z3 is N; Y1 is N; Y2 is C; one of Z1 and Z2 is CR₂ and the other is C; W₁ is N—R₁; W₂ is C═O; W₃ is CR4R4; and t=1.
69. In formula 16-B Z3 is N; Y1 is N; Y2 is C; one of Z1 and Z2 is CR₂ and the other is C; W₁ is C═O; W₂ is N—R₁; W₃ is CH₂; and t=1.

The more preferred embodiments of the formula 16-C are:
70. In formula 16-C Z1, Z2, Z3 and Z4 are independently CR₂; one of Z1-Z4 is carbon and is bonded to the remainder of the molecule; Y₁ and Y₂ are C; t=1 to 3; and W₁, W₂ and W₃ are independently CR4R4, S, SO, SO₂, O, or N—R₁.
71. In formula 16-C Z1, Z2, Z3 and Z4 are independently CR₂ and one of Z1-Z4 is carbon and is bonded to the remainder of the molecule; Y₁ and Y₂=C or N; t=1 to 3; W₁, W₂ and W₃ are independently CR4R4, S, SO, SO₂, O, or N—R₁.
72. In formula 16-C Z1, Z2, Z3 and Z4 are independently CR₂; Y₁ and Y₂ are N; t=1 to 3; W₁, W₂ and W₃ are independently CR4R4, S, SO, SO₂, O, or N—R₁.
73. In formula 16-C Z1 is N and Z2, Z3 and Z4 are independently CR₂; Y₁ and Y₂ are C; t=1 to 3; W₁, W₂ and W₃ are independently CR4R4, S, SO, SO₂, O, or N—R₁.
74. In formula 16-C Z1 is N and Z2, Z3 and Z4 are independently CR₂; one of Z1-Z4 is carbon and is bonded to the remainder of the molecule; Y₁ is C; Y₂ is N; t=1 to 3; and W₁, W₂ and W₃ are independently CR4R4, S, SO, SO₂, O, or N—R₁.
75. In formula 16-C Z2=N and Z1, Z3 and Z4 are independently CR₂; one of Z1-Z4 is carbon and is bonded to the remainder of the molecule; Y₁ and Y₂ are C; t=1 to 3; and W₁, W₂ and W₃ are independently CR4R4, S, SO, SO₂, O, or N—R₁.
76. In formula 16-C Z2 is N and Z1, Z3 and Z4 are independently CR₂; one of Z1-Z4 is carbon and is bonded to the remainder of the molecule; Y₁ is C; Y₂ is N; t=1 to 3; and W₁, W₂ and W₃ are independently CR4R4, S, SO, SO₂, O, or N—R₁.
77. In formula 16-C Z3 is N; Z1, Z2 and Z4 are independently CR₂; one of Z1-Z4 is carbon and is bonded to the remainder of the molecule; Y₁ and Y₂ are C; t=1 to 3; and W₁, W₂ and W₃ are independently CR4R4, S, SO, SO₂, O, or N—R₁.
78. In formula 16-C Z3 is N and Z1, Z2 and Z4 are independently CR₂; one of Z1-Z4 is carbon and is bonded to the remainder of the molecule; Y₁ is C and Y₂ is N; t=1 to 3; and W₁, W₂ and W₃ are independently CR4R4, S, SO, SO₂, O, or N—R₁.
79. In formula 16-C Z4 is N and Z1, Z2 and Z3 are independently CR₂; one of Z1-Z4 is carbon and is bonded to the remainder of the molecule; Y₁ and Y₂ are C; t=1 to 3; and W₁, W₂ and W₃ are independently CR4R4, S, SO, SO₂, O, or N—R₁.
80. In formula 16-C Z4 is N and Z1, Z2 and Z3 are independently CR₂; one of Z1-Z4 is carbon and is bonded to the remainder of the molecule; Y₁ is N; Y₂ is C; t=1 to 3; and W₁, W₂ and W₃ are independently CR4R4, S, SO, SO₂, O, or N—R₁.
81. In formula 16-C Z1 is N and Z2, Z3 and Z4 are independently CR₂; one of Z1-Z4 is carbon and is bonded to the remainder of the molecule; Y₁ and Y₂ are C; t=1 to 3; and W₁, W₂ and W₃ are independently CR4R4, S, SO, SO₂, O, or N—R₁.
82. In formula 16-C Z1 and Z2 are N and Z3 or Z4 are independently CR₂; one of Z1-Z4 is carbon and is bonded to the remainder of the molecule; Y₁ is C; Y₂ is N; t=1 to 3; and W₁, W₂ and W₃ are independently CR4R4, S, SO, SO₂, O, or N—R₁.
83. In formula 16-C Z1 and Z3 are N and Z2 or Z4 are independently CR₂; one of Z1-Z4 is carbon and is bonded to the remainder of the molecule; Y₁ is C; Y₂ is N; t=1 to 3; and W₁, W₂ and W₃ are independently CR4R4, S, SO, SO₂, O, or N—R₁.
84. In formula 16-C Z1 and Z4 are N and Z2 or Z3 are independently CR₂; one of Z1-Z4 is carbon and is bonded to the remainder of the molecule; Y₁ is N; Y₂ is C; t=1 to 3; and W₁, W₂ and W₃ are independently CR4R4, S, SO, SO₂, O, or N—R₁.
85. In formula 16-C Z1, Z2, Z3 are N and Z4 is carbon and is bonded to the remainder of the molecule; Y₁ is C; Y₂ is N; t=1 to 3; and W₁, W₂ and W₃ are independently CR4R4, S, SO, SO₂, O, or N—R₁.
86. In formula 16-C Z1, Z3 and Z4 are N and Z2 is carbon and is bonded to the remainder of the molecule; Y₁ and Y₂ are C; t=1 to 3; and W₁, W₂ and W₃ are independently CR4R4, S, SO, SO₂, O, or N—R₁.
87. In formula 16-C Z1, Z2 and Z4 are N and Z3 is carbon and is bonded to the remainder of the molecule; Y₁ and Y₂ are C and t=1 to 3; and W₁, W₂ and W₃ are independently CR4R4, S, SO, SO₂, O, or N—R₁.
88. In formula 16-C Z2, Z3, Z4 are N and Z1 is carbon and is bonded to the remainder of the molecule; Y₁ and Y₂ are C and t=1 to 3; and W₁, W₂ and W₃ are independently CR4R4, S, SO, SO₂, O, or N—R₁.

The most preferred compounds of the present invention are:
1. 7-[(5-benzyl-4,5,6,7-tetrahydrothieno[3,2-c]pyridin-2-yl)]-4,7-dihydro-[1,4]thiazepine-3,6-dicarboxylicacid 6-methyl ester.
2. 7-(7-Methyl-5,6,7,8-tetrahydroimidazo[1,2-a]pyrazin-2-yl)-4,7-dihydro-[1,4]thiazepine-3,6-dicarboxylicacid 6-methyl ester.
3. 7-(5,6,7,8-tetrahydroimidazo[1,2-a]pyrazin-2-yl)-4,7-dihydro-[1,4]thiazepine-3,6-dicarboxylicacid 6-methyl ester.
4. 7-{[5-(4-methoxybenzyl)-4,5,6,7-tetrahydrothieno[3,2-c]pyridin-2-yl)]4,7-dihydro-[1,4]thiazepine-3,6-dicarboxylicacid 6-methyl ester.
5. 7-(5,6-dihydro-8H-imidazo[2,1-c][1,4]thiazin-2-yl) 4,7-dihydro-[1,4]thiazepine-3,6-dicarboxylicacid 6-methyl ester.
6. 7-(6,7-Dihydro-5H-pyrrolo[1,2-a]imidazol-2-yl)-4,7-dihydro-[1,4]thiazepine-3,6-dicarboxylicacid 6-methyl ester.
7. 7-(5,6-Dihydro-8H-imidazo[2,1-c][1,4]oxazin-2-yl)-4,7-dihydro-[1,4]thiazepine-3,6-dicarboxylicacid 6-methyl ester.
8. 7-(5,6-Dihydro-4H-pyrrolo[1,2-b]pyrazol-2-yl)-4,7-dihydro-[1,4]thiazepine-3,6-dicarboxylicacid 6-methyl ester.
9. 7-(4,5,6,7-tetrahydropyrazolo[1,5-a]pyridin-2-yl)-4,7-dihydro-[1,4]thiazepine-3,6-dicarboxylicacid 6-methyl ester.
10. 7-(7-Methyl-6-oxo-5,6,7,8-tetrahydro-imidazo[1,2-a]pyrazin-2-yl)-4,7-dihydro-[1,4]thiazepine-3,6-dicarboxylicacid 6-methyl ester.
11. 7-(6,7-Dihydro-4H-pyrazolo[5,1-c][1,4]thiazin-2-ylmethyl)-4,7-dihydro-[1,4]thiazepine-3,6-dicarboxylicacid 6-methyl ester.
12. 7-(4H-5-thia-1,6a-diazapentalen-2-ylmethyl)-4,7-dihydro-[1,4]thiazepine-3,6-dicarboxylicacid 6-methyl ester.
13. 7-(7H-Imidazo[1,2-c]thiazol-2-ylmethyl)-4,7-dihydro-[1,4]thiazepine-3,6-dicarboxylicacid 6-methyl ester.
14. 7-[(4-oxo-6,7-dihydro-4H-pyrazolo[5,1-c][1,4]oxazin-2-yl)methyl)-4,7-dihydro-[1,4]thiazepine-3,6-dicarboxylicacid 6-methyl ester.
15. 7-(6,7-Dihydro-4H-thieno[3,2-c]pyran-2-ylmethylene)-4,7-dihydro-[1,4]thiazepine-3,6-dicarboxylicacid 6-methyl ester.
16. 7-(6,7-Dihydro-4H-thieno[3,2-c]thiopyran-2-ylmethyl)-4,7-dihydro-[1,4]thiazepine-3,6-dicarboxylicacid 6-methyl ester.
17. 7-(5-Methyl-4,5,6,7-tetrahydro-thieno[3,2-c]pyridin-2-ylmethyl)-4,7-dihydro-[1,4]thiazepine-3,6-dicarboxylic acid 6-methyl ester.
18. 7-(6-ethoxycarbonyl-4,5,6,7-tetrahydro-thieno[2,3-c]pyridin-2-yl)-4,7-dihydro-[1,4]thiazepine-3,6-dicarboxylic acid 6-methyl ester.
19. 7-(6,7,8,9-tetrahydro-5H-imidazo[1,2-a]azepin-2-ylmethyl)-4,7-dihydro-[1,4]thiazepine-3,6-dicarboxylic acid 6-methyl ester.
20. 7-(7-Benzyl-5,6,7,8-tetrahydroimidazo[1,2-a]pyrazin-2-ylmethyl)-4,7-dihydro-[1,4]thiazepine-3,6-dicarboxylic acid 6-methyl ester.
21. 7-{[5-(pyridin-3-ylmethyl)-4,5,6,7-tetrahydrothieno[3,2-c]pyridin-2-yl)]methyl}-4,7-dihydro-[1,4]thiazepine-3,6-dicarboxylic acid 6-methyl ester.
22. 7-{[5-(pyridin-3-ylcarbonyl)-4,5,6,7-tetrahydrothieno[3,2-c]pyridin-2-Y₇)]methyl}-4,7-dihydro-[1,4]thiazepine-3,6-dicarboxylic acid 6-methyl ester.
23. 7-{[5-(phenylacetyl)-4,5,6,7-tetrahydrothieno[3,2-c]pyridin-2-yl)]methyl}-4,7-dihydro-[1,4]thiazepine-3,6-dicarboxylic acid 6-methyl ester.
24. 7-(5,5-Dioxo-4,5,6,7-tetrahydro-5λ⁶-pyrazolo[5,1-c][1,4]thiazin-2-ylmethyl)-4,7-dihydro-[1,4]thiazepine-3,6-dicarboxylic acid 6-methyl ester.
25. 7-(4,5,6,7-tetrahydropyrazolo[1,5-a]pyrazin-2-ylmethyl)-4,7-dihydro-[1,4]thiazepine-3,6-dicarboxylic acid 6-methyl ester.
26. 7-(5,5-Dimethyl-4H-1,6a-diazapentalen-2-ylmethyl)-4,7-dihydro-[1,4]thiazepine-3, 6-dicarboxylic acid 6-methyl ester.
27. 7-(5,6-Dihydro-4H-cyclopenta[b]furan-2-ylmethyl)-4,7-dihydro-[1,4]thiazepine-3,6-dicarboxylic acid 6-methyl ester.
28. 7-(4,5-Dihydro-6-thia-1,7a-diazainden-2-ylmethyl)-4,7-dihydro-[1,4]thiazepine-3,6-dicarboxylic acid 6-methyl ester.
29. 7-(6,6-Dimethyl-5,6,7,8-tetrahydroimidazo[1,2-a]pyrizin-2-ylmethyl)-4,7-dihydro-[1,4]thiazepine-3,6-dicarboxylic acid 6-methyl ester.
30. 7-(5,6-Dihydro-8-H-imidazo[2,1-c][1,4]thiazin-3-ylmethyl)-4,7-dihydro-[1,4]thiazepine-3,6-dicarboxylic acid 6-methyl ester.
31. 7-(4H-5-thia-1,6a-diazapentalen-2-ylmethyl)-4,7-dihydro-[1,4]thiazepine-3,6-dicarboxylic acid 6-methyl ester.
32. 7-(2,3-Dihydropyrazolo[5,1-b]thiazol-6-ylmethyl)-4,7-dihydro-[1,4]thiazepine-3,6-dicarboxylic acid 6-methyl ester.
33. 7-(2,3-Dihydropyrazolo[5,1-b]oxazol-6-ylmethyl)-4,7-dihydro-[1,4]thiazepine-3,6-dicarboxylic acid 6-methyl ester.
34.7-[(5-acetyl-4,5,6,7-tetrahydrothieno[3,2-c]pyridin-2-yl)methyl]-4,7-dihydro-[1,4]thiazepine-3,6-dicarboxylic acid 6-methyl ester.
35. 7-(6,7-dihydro-4H-pyrazolo[5,1-c][1,4]oxazin-2-ylmethyl)-4,7-dihydro-[1,4]thiazepine-3,6-dicarboxylic acid 6-methyl ester.
36. 7-(6,7-5H-Dihydropyrazolo[5,1-b]oxazin-2-ylmethyl)-4,7-dihydro-[1,4]thiazepine-3,6-dicarboxylic acid 6-methyl ester.
37. 7-(Imidazo[2,1-b][1,3]benzothiazol-2-ylmethyl)-4,7-dihydro-[1,4]thiazepine-3,6-dicarboxylic acid 6-methyl ester.
38. 7-[(7-methoxyimidazo[2,1-b][1,3]benzothiazol-2-ylmethyl)-4,7-dihydro-[1,4]thiazepine-3,6-dicarboxylic acid 6-methyl ester.
39. 7-[(7-chloroimidazo[2,1-b][1,3]benzothiazol-2-ylmethyl)-4,7-dihydro-[1,4]thiazepine-3,6-dicarboxylic acid 6-methyl ester.
40. 7-Imidazo[1,2-a]quinolin-2-ylmethyl-4,7-dihydro-[1,4]thiazepine-3,6-dicarboxylic acid 6-methyl ester.
41. 7-(6,7-dihydro-5H-cyclopenta[d]imidazo[2,1-b][1,3]thiazol-2-ylmethylene)-4,7-dihydro-[1,4]thiazepine-3,6-dicarboxylic acid 6-methyl ester.
42. 7-(Imidazo[1,2-a]quinoxaline-2-ylmethyl)-4,7-dihydro-[1,4]thiazepine-3,6-dicarboxylic acid 6-methyl ester.
43. 7-[(7-methylimidazo[2,1-b][1,3]benzothiazol-2-ylmethyl)-4,7-dihydro-[1,4]thiazepine-3,6-dicarboxylic acid 6-methyl ester.
44. 7-(4,5,6,7-tetrahydro-1,3a,3b,8-tetraaza-cyclopenta[a]indene-2-ylmethyl)-4,7-dihydro-[1,4]thiazepine-3,6-dicarboxylic acid 6-methyl ester.
45. 7-[(10-benzyl-11-oxo-10,11-dihydrodibenzo[b,f][1,4]oxazepin-8-yl)methyl]-4,7-dihydro-[1,4]thiazepine-3,6-dicarboxylic acid 6-methyl ester.
46. 7-(5-ethoxy-7,8-dihydro-6H-3,4,8b-triaza-as-indacen-2-ylmethylene)-4,7-dihydro-[1,4]thiazepine-3,6-dicarboxylic acid 6-methyl ester.
47. 7-(4H, 10H-pyrazolo[5,1-c][1,4]benzoxazepin-2-ylmethyl)-4,7-dihydro-[1,4]thiazepine-3,6-dicarboxylic acid 6-methyl ester.
48. 7-(5H-Imidazo[2,1-a]isoindol-2-ylmethyl)-4,7-dihydro-[1,4]thiazepine-3,6-dicarboxylic acid 6-methyl ester.
49. 7-[(5-methylimidazo[1,2-b][1,3]benzothiazol-2-ylmethyl)]-4,7-dihydro-[1,4]thiazepine-3,6-dicarboxylic acid 6-methyl ester.
50. 7-[(7-fluoroimidazo[2,1-b][1,3]benzothiazol-2-ylmethyl)-4,7-dihydro-[1,4]thiazepine-3,6-dicarboxylic acid 6-methyl ester.
51. 7-(5,8-dihydro-6H-imidazo[2,1-b]pyrano[4,3-d][1,3]thiazol-2-ylmethyl)-4,7-dihydro-[1,4]thiazepine-3,6-dicarboxylic acid 6-methyl ester.
52. 7-(imidazo[2,1-b]benzothiazol-7-ylmethyl)-4,7-dihydro-[1,4]thiazepine-3,6-dicarboxylic acid 6-methyl ester.
53. 7-([1,3]thiazolo[3,2-a]benzimidazol-2-ylmethyl)-4,7-dihydro-[1,4]thiazepine-3,6-dicarboxylic acid 6-methyl ester.
54. 7-(7,8-dihydro-6H-cyclopenta[3,4]pyrazolo[5,1-b][1,3]thiazol-2-ylmethyl)-4,7-dihydro-[1,4]thiazepine-3,6-dicarboxylic acid 6-methyl ester.
55. 7-(5,6,7,8-tetrahydroimidazo[2,1-b][1,3]benzothiazol-2-ylmethyl)-4,7-dihydro-[1,4]thiazepine-3,6-dicarboxylic acid 6-methyl ester.
56. 7-[(9-methyl-9H-imidazo[1,2-a]benzimidazol-2-yl)methyl]-4,7-dihydro-[1,4]thiazepine-3,6-dicarboxylic acid 6-methyl ester.
57. 7-(4H-thieno[2′,3′:4,5]thiopyrano[2,3-b]pyridin-2-ylmethyl)-4,7-dihydro-[1,4]thiazepine-3,6-dicarboxylic acid 6-methyl ester.
58. 7-[(5-methyl-7,8-dihydro-6H-cyclopenta[e][1,2,4]triazolo[1,5-a]pyrimidin-2-yl)methyl]-4,7-dihydro-[1,4]thiazepine-3,6-dicarboxylic acid 6-methyl ester.
59. 7-{[7-(ethoxycarbonyl)-6,7,8,9-tetrahydropyrido[3,4-e][1,2,4]triazolo[1,5-a]pyrimidin-2-yl]methyl}-4,7-dihydro-[1,4]thiazepine-3,6-dicarboxylic acid 6-methyl ester.
60. 7-(8′,9′-dihydro-6′H-spiro[1,3-dioxolane-2,7′-[1,2,4]triazolo[1,5-a]quinazolin]-2′-ylmethyl)-4,7-dihydro-[1,4]thiazepine-3,6-dicarboxylic acid 6-methyl ester.
61. 7-[(5-methyl-6,7,8,9-tetrahydro[1,2,4]triazolo[1,5-a]quinazolin-2-yl)methyl]-4,7-dihydro-[1,4]thiazepine-3,6-dicarboxylic acid 6-methyl ester.
62. 7-[(5-methoxy-7,8-dihydro-6H-cyclopenta[e]imidazo[1,2-a]pyrimidin-2-yl)methyl]-4,7-dihydro-[1,4]thiazepine-3,6-dicarboxylic acid 6-methyl ester.
63. 7-({5-[2-(benzyloxy)ethoxy]-7,8-dihydro-6H-cyclopenta[e]imidazo[1,2-a]pyrimidin-2-yl}methyl)-4,7-dihydro-[1,4]thiazepine-3,6-dicarboxylic acid 6-methyl ester.
64. 7-(2,3-dihydro[1,3]thiazolo[3,2-a]benzimidazol-6-ylmethyl)-4,7-dihydro-[1,4]thiazepine-3,6-dicarboxylic acid 6-methyl ester.
65. 7-(3,4-dihydro-2H-[1,3]thiazino[3,2-a]benzimidazol-7-ylmethyl)-4,7-dihydro-[1,4]thiazepine-3,6-dicarboxylic acid 6-methyl ester.
66. 7-([1,3]thiazolo[3,2-a]benzimidazol-6-ylmethyl)-4,7-dihydro-[1,4]thiazepine-3,6-dicarboxylic acid 6-methyl ester.
67. 7-(7,8-dihydro-5H-pyrano[4,3-d]pyrazolo[5,1-b][1,3]oxazol-2-ylmethyl)-4,7-dihydro-[1,4]thiazepine-3,6-dicarboxylic acid 6-methyl ester.
68. 7-(5,6,7,8-tetrahydropyrazolo[5,1-b][1,3]benzoxazol-2-ylmethyl)-4,7-dihydro-[1,4]thiazepine-3,6-dicarboxylic acid 6-methyl ester.
69. 7-{[6-(Ethoxycarbonyl)-5,6,7,8-tetrahydropyrazolo[5′,1′:2,3][1,3]oxazolo[5,4-c]pyridin-2-yl]methyl}-4,7-dihydro-[1,4]thiazepine-3,6-dicarboxylic acid 6-methyl ester.

Pharmaceutically acceptable salts are those salts which may be administered or provided to a warm blooded animal, preferably sodium, potassium or calcium alkaline earth metal salts.

The compounds according to the present invention have β-lactamase inhibitory and antibacterial properties and are useful for the treatment of infections in humans and animals. It should be noted that the compounds of the present invention, when used in combination with β-lactam antibiotics will result in the increased antibacterial activity (synergistic effect) against class-A and class-C producing organisms. β-Lactam antibiotics include penicillin antibiotics such as piperacillin, amoxycillin, ticarcillin, benzylpenicillins, ampicillin, sulbenicillin, other known penicillins and cephalosporins such as cefatrizine, cephaloridine, cephalothin, cefazolin, cephalexin, cephradine, other known cephalosporins, aztreonam and latamoxef (Moxalactam). Most preferably compounds of this present invention are used with piperacillin or amoxicillin which has a broad spectrum of activity against Gram positive and Gram negative pathogens.

The compounds of the present invention may be provided prior to, simultaneously with, or subsequent to a β-lactam antibiotic (“co-administration”). By “provided”, it is intended to include administering the compound directly or in vivo, e.g. pro-drugs. When the compounds of the present invention are co-administered with a β-lactam antibiotic, the ratio of the amount of the compound to the amount of the β-lactam antibiotic may vary in a wide range. The ratio of β-lactam antibiotic to β-lactamase inhibitor may vary from 1:1 to 100:1. Preferably the ratio of the β-lactam antibiotic to β-lactamase inhibitor is less than 10:1. The composition of the present invention may be in a form suitable for oral (PO), intravenous (IV) or topical administration. The compositions of the invention may be in a form of tablets, capsules, creams, syrups, suspension, sterile solutions suitable for injection or infusion. Preferably, the compounds of the present invention are co-administered with piperacillin intravenously or amoxicillin intravenously or orally.

A compound's structural formula includes any tautomers, any stereoisomers (except where stereochemistry is clearly noted) and any crystalline forms.

Chemical Definitions

The term alkyl means both straight and branched chain alkyl moieties of 1-12 carbons, preferably of 1-6 carbon atoms.

The term alkenyl means both straight and branched alkenyl moieties of 2-8 carbon atoms containing at least one double bond, and no triple bond, preferably the alkenyl moiety has 1 or two double bonds. Such alkenyl moieties may exist in the E or Z conformations; the compounds of this invention include both conformations. In the case of alkenyl, hetero atoms such as O, S or N—R₁ should not be present on the carbon that is bonded to a double bond;

The term alkynyl includes both straight chain and branched alkynyl moieties containing 2-6 carbon atoms containing at least one triple bond, preferably the alkynyl moiety has one or two triple bonds. In the case of alkynyl, hetero atoms such as O, S or N—R₁ should not be present on the carbon that is bonded to a double or triple bond;

The term cycloalkyl refers to a alicyclic hydrocarbon group having 3-7 carbon atoms.

The term perfluoroalkyl is used herein to refer to both straight- and branched-chain saturated aliphatic hydrocarbon groups having at least one carbon atom and two or more fluorine atoms. Examples include CF₃, CH₂CF₃, CF₂CF₃ and CH(CF₃)₂.

The term halogen is defined as Cl, Br, F, and 1.

If alkyl, alkenyl, alkynyl, or cycloalkyl is “optionally substituted”, one or two of the following are possible substituents: nitro, -aryl, -heteroaryl, alkoxyoarbonyl-, -alkoxy, -alkoxy-alkyl, alkyl-O—C2-C4alkyl-O—, -cyano, -halogen, -hydroxy, —N—R₆R₇, —COOH, —COO-alkyl, -trifluoromethyl, -trifluoromethoxy, arylalkyl, alkylaryl, R₆R₇N-alkyl-, HO—C1-C6-alkyl-, alkoxyalkyl-, alkyl-S—, —SO₂N—R₆R₇, —SO₂NHR₆, —CO₂H, CONR₆R₇, aryl-O—, heteroaryl-O—, —S(O)ₛ-aryl (where s=0-2), -alkyl-O-alkyl-NR₆R₇, -alkyl-aryl-O-alkylN—R₆R₇, C1-C6alkyl, alkenyl, alkynyl, cycloalkyl, alkoxy-alkyl-O—, R₆R₇N-alkyl-, and —S(O)ₛ-heteroaryl (where s=0-2); Preferred substitutents for alkyl, alkenyl, alkynyl, and cycloalkyl include: halogen, nitro, aryl, heteroaryl, alkoxycarbonyl-, alkoxy, -alkoxy-alkyl, -cyano, hydroxy, and —N—R₆R₇.

Aryl is defined as an aromatic hydrocarbon moiety selected from the group: phenyl, α-naphthyl, β-naphthyl, biphenyl, anthryl, tetrahydronaphthyl, fluorenyl, indanyl, biphenylenyl, acenaphthenyl, groups. Preferred aryl groups are phenyl and biphenyl.

Heteroaryl is defined as a aromatic heterocyclic ring system (monocyclic or bicyclic) where the heteroaryl moieties are selected from: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline; (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Preferred heteroaryl groups are furan, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, quinoline, isoquinoline, and naphthyridine.

If aryl or heteroaryl is ‘optionally substituted’, one or two of the following are possible substituents: nitro, -aryl, -heteroaryl, alkoxycarbonyl-, -alkoxy, -alkoxy-alkyl, alkyl-O—C2-C4alkyl-O—, -cyano, -halogen, -hydroxy, —N—R₆R₇, -trifluoromethyl, -trifluoromethoxy, arylalkyl, alkylaryl, R₆R₇N-alkyl-, HO—C1-C6-alkyl-, alkoxyalkyl-, alkyl-S—, —SO₂N—R₆R₇, —SO₂NHR₆, —CO₂H, CONR₆R₇, aryl-O—, heteroaryl-O—, —S(O)_{S}-aryl (where s=0-2), -alkyl-O-alkyl-NR₆R₇, -alkyl-aryl-O-alkylN—R₆R₇, C1-C6alkyl, alkenyl, alkynyl, cycloalkyl, alkoxy-alkyl-O—, R₆R₇N-alkyl-, and —S(O)ₛ-heteroaryl (where s=0-2); Preferred substituents for aryl and heteroaryl include: alkyl, halogen, —N—R₆R₇, trifluoromethyl, -trifluoromethoxy, arylalkyl, and alkylaryl.

Arylalkyl is defined as Aryl-C1-C6alkyl-; Arylalkyl moieties include benzyl, 1-phenylethyl, 2-phenylethyl, 3-phenylpropyl, 2-phenylpropyl and the like. The term ‘optionally substituted’ refers to unsubstituted or substituted with 1 or 2 substituents on the alkyl or aryl moiety as defined above.

Alkylaryl is defined as C1-C6alkyl-aryl-. The term ‘optionally substituted’ refers to unsubstituted or substituted with 1 or 2 substituents on the aryl or alkyl moiety as defined above.

Heteroaryl-C1-C6-alkyl is defined as a heteroaryl substituted alkyl moiety wherein the alkyl chain is 1-6 carbon atoms (straight or branched). Alkyl heteroaryl moieties include Heteroaryl-(CH₂)₁₋₆— and the like. The term ‘optionally substituted’ refers to unsubstituted or substituted with 1 or 2 substituents on the alkyl or heteroaryl moiety as defined above;

C1-C6 alkylheteroaryl is defined an alkyl chain of 1-6 carbon atoms (straight or branched) attached to a heteroaryl moiety, which is bonded to the rest of the molecule. For example C1-C6-alkyl-Heteroaryl-. The term ‘optionally substituted’ refers to unsubstituted or substituted with 1 or 2 substituents on the alkyl or heteroaryl moiety as defined above;

Saturated or partially saturated heterocycles groups are defined as heterocyclic rings selected from the moieties; aziridinyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl. Preferred saturated or partially saturated heterocycles include: aziridinyl, azetidinyl, 1,4-dioxanyl, hexahydroazepinyl, piperazinyl, piperidinyl, pyrrolidinyl, morpholinyl, thiomorpholinyl, tetrahydroquinolinyl, tetrahydroisoquinolinyl, dihydroimidazolyl, and dihydroisooxazolyl.

C1-C6 alkyl mono or bicyclic saturated or partially saturated heterocycles is defined as an alkyl group (straight or branched) of C1-C6 attached to a heterocycles (which is defined before) through a carbon atom or a nitrogen atom and the other end of the alkyl chain attached to the rest of the molecule. The terms ‘optionally substituted’ refers to unsubstituted or substituted with 1 or 2 substituents present on the alkyl or heterocyclic portion of the molecule, as defined before;

Arylalkyloxyalkyl is defined as Aryl-C1-C6alkyl-O—C1-C6alkyl-. The term ‘optionally substituted’ refers to unsubstituted or substituted with 1 or 2 substituents present on the alkyl and/or aryl portions as defined before;

Alkyloxyalkyl is defined as C1-C6 alkyl-O—C1-C6alkyl-. The term ‘optionally substituted’ refers to unsubstituted or substituted with 1 or 2 substituents present at the alkyl moiety as defined before;

Aryloxyalkyl is defined as Aryl-O—C1-C6 alkyl-. The term ‘optionally substituted’ refers to unsubstituted or substituted with 1 or 2 substituents present at the alkyl or aryl moiety as defined before;

Heteroarylalkyloxyalkyl is defined as Heteroaryl-C1-C6alkyl-O—C1-C6alkyl-. The term ‘optionally substituted’ refers to unsubstituted or substituted with 1 or 2 substituents present on the alkyl or heteroaryl moiety as defined before;

Aryloxyaryl is defined as Aryl-O-Aryl-. The term ‘optionally substituted’ refers to unsubstituted or substituted with 1 or 2 substituents present on the aryl moiety as defined before;

Aryloxyheteroaryl is defined as Aryl-O-Heteroaryl- or -Aryl-O-Heteroaryl; In this definition either the aryl moiety or the heteroaryl moiety can be attached to the remaining portion of the molecule; The term ‘optionally substituted’ refers to unsubstituted or substituted with 1 or 2 substituents present on the aryl moiety or on the heteroaryl moiety as defined before;

Alkyl aryloxyaryl is defined as Aryl-O-Aryl-C1-C6alkyl-; The term ‘optionally substituted’ refers to unsubstituted or substituted with 1 or 2 substituents present at the aryl moiety as defined before;

Alkylaryloxyheteroaryl is defined as Heteroaryl-O-Aryl-C1-C6alkyl-; The term ‘optionally substituted’ refers to unsubstituted or substituted with 1 or 2 substituents present on the aryl moiety or on the hetroaryl moiety as defined before;

Alkylaryloxyalkylamine is defined as R₆R₇N—C1-C6alkyl-O-Aryl-C1-C6alkyl-; The terms ‘optionally substituted’ refers to unsubstituted or substituted with 1 or 2 substituents present on the alkyl or aryl moiety as defined before; R₆ and R₇ as defined before;

Alkoxycarbonyl is defined as C1-C6alkyl-O—C═O—; The term ‘optionally substituted’ refers to unsubstituted or substituted with 1 or 2 substituents present on the alkyl portion of the alkoxy moiety as defined before;

Aryloxycarbonyl is defined as Aryl-O—C═O—; The term ‘optionally substituted’ refers to unsubstituted or substituted with 1 or 2 substituents present at the aryl moiety as defined before;

Heteroaryloxy carbonyl is defined as Heteroaryl-O—C═O—; The term ‘optionally substituted’ refers to unsubstituted or substituted with 1 or 2 substituents present at the heteroaryl moiety as defined before;

Alkoxy is defined as C1-C6alkyl-O—; The terms ‘optionally substituted’ refers to unsubstituted or substituted with 1 or 2 substituents present at the alkyl moiety as defined before;

Aryloxy is defined as Aryl-O—; The term ‘optionally substituted’ refers to unsubstituted or substituted with 1 or 2 substituents present at the aryl moiety as defined before;

Heteroaryloxy is defined as Heteroaryl-O—; The term ‘optionally substituted’ refers to unsubstituted or substituted with 1 or 2 substituents present at the heteroaryl moiety as defined before;

Alkenyloxy is defined as C3-C6 alkene-O—; Example allyl-O—, but-2-ene-O or like moieties; The term ‘optionally substituted’ refers to unsubstituted or substituted with 1 or 2 substituents present at the alkene moiety as defined before, with the proviso that no hetero atom such as O, S or N—R₁ is present on the carbon atom, which is attached to a double bond;

Alkynyloxy is defined as C3-C6alkyne-O—; Example CH triple bond C—CH₂—O—, or like moieties; The term ‘optionally substituted’ refers to unsubstituted or substituted with 1 or 2 substituents present at the alkyne moiety as defined before, with the proviso that no hetero atom such as O, S or N—R₁ is present on a carbon atom which is attached to a double or triple bond;

Alkylaminoalkoxy is defined as R₆R₇N—C1-C6-alkyl-O—C1-C6-alkyl-, where the terminal alkyl group attached to the oxygen is connected to the rest of the molecule; The terms R₆ and R₇ are defined above; The term ‘optionally substituted’ refers to unsubstituted or substituted with 1 or 2 substituents present at the alkyl moiety as defined before;

Alkylenedioxy is defined as —O—CH₂—O— or —O—(CH₂)₂—O—;

Aryloxyalkylamine is defined as R₆R₇N—C1-C6-alkyl-O-Aryl-, where the aryl is attached to the rest of the molecule; The term ‘optionally substituted’ refers to unsubstituted or substituted with 1 or 2 substituents present at the alkyl or aryl moiety as defined before;

Arylalkenyl is defined as Aryl-C2-C8alkene—, with the proviso that no hetero atom such as O, S or N—R₁ is present on the carbon atom, which is attached to a double bond; The term ‘optionally substituted’ refers to unsubstituted or substituted with 1 or 2 substituents present on the alkene or aryl moiety as defined before;

Heteroaryloxyalkyl is defined as Heteroaryl-O—C1-C6alkyl-; The term ‘optionally substituted’ refers to unsubstituted or substituted with 1 or 2 substituents present at the heteroaryl moiety as defined before;

Heteroaryloxyaryl is defined as Heteroaryl-O-aryl-, where the aryl moiety is attached to the rest of the molecule; The term ‘optionally substituted’ refers to unsubstituted or substituted with 1 or 2 substituents present at the heteroaryl moiety or the aryl moiety as defined before;

Alkoxy, alkoxyalkyl, alkoxyalkyloxy and alkylthioalkyloxy are moieties wherein the alkyl chain is 1-6 carbon atoms (straight or branched). Aryloxy, heteroaryloxy, arylthio and heteroarylthio are moieties wherein the aryl and heteroaryl groups are as herein before defined. Arylalkyloxy, heteroarylalkyloxy, arylalkylthio and heteroarylalkylthio are moieties wherein the aryl and heteroaryl groups are as herein before defined and wherein the alkyl chain is 1-6 carbons (straight or branched). Aryloxyalkyl, heteroaryloxyalkyl, aryloxyalkyloxy and heteroaryloxyalkyloxy are substituents wherein the alkyl radical is 1-6 carbon atoms. The terms monoalkylamino and dialkylamino refer to moieties with one or two alkyl groups wherein the alkyl chain is 1-6 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1-3 carbon atoms.

IC₅₀ Determination for the Inhibitors

β-Lactamase inhibitory activity of the inhibitors was determined spectrophotometrically as described by Bush et al., [Bush, K., Macalintal, C., Rasmussen, B. A., Lee, V. and Yang, Y. Antimicrobial Agents and Chemotherapy 1993, 37, 851]. Homogeneously purified class A β-lactamases TEM-1 from E. coli and Imi-1 from Enterobacter cloacae, class B enzyme CcrA from Bacteroides fragilis and class C enzyme AmpC from Enterobacter cloaca were employed in the assay. The enzyme concentrations for TEM-1, Imi-1, CcrA and AmpC were 4.3, 7.1, 1.2 and 2.1 nM, respectively. A wide range of inhibitor concentrations were prepared in 50 mM PO₄, pH 7.0 to include the possible IC₅₀ values. The substrate used to initiate the enzyme reaction was nitrocefin at 50 μg/ml in the same buffer as the inhibitor. Initially the enzyme and inhibitor (20 μl each) were preincubated for 10 minutes at 25° C. prior to the addition of 160 μl volume of nitrocefin. Initial rates of hydrolysis were monitored for 5 minutes at 495 nm using a Molecular Devices Spectra Max 250 with kinetic protocol of SoftMax Program. Readings from the Spectra Max 250 were exported and transferred to Microsoft Excel. The percent of inhibition of each inhibitor concentration was calculated based on the control enzyme activity. The inhibitor concentration that caused a 50% reduction in the enzymatic activity (IC₅₀) was determined graphically.

Antimicrobial susceptibility testing. The in vitro activities of the antibiotics were determined by the microbroth dilution method as recommended by the National Committee for Clinical Laboratory Standards (NCCLS). (NCCLS. 2000. Methods for Dilution Antimicrobial Susceptibility Tests for Bacteria That Grow Aerobically; Approved Standards: M7-A5, vol. 19. National Committee for Clinical Laboratory Standards, Villanova, Pa.). Mueller-Hinton II broth (MHBII)(BBL Cockeysville, Md.), was used for the testing procedure. Microtiter plates containing 50 μl per well of two-fold serial dilutions of piperacillin combined with a constant amount (4 ug/ml) of a B-lactamase inhibitor were inoculated with 50 μl of inoculum to yield the appropriate density (10⁵ CFU/ml) in 100·I final volume. The plates were incubated for 18-22 hours at 35° C. in ambient air. The minimal inhibitory concentration (MIC) for all isolates was defined as the lowest concentration of antimicrobial agent that completely inhibits the growth of the organism as detected by the unaided eye.

In Vivo Antibacterial Protection

Materials:

Animals:

Female mice strain CD-1, approximately 18-22 grams, were received from Charles River Laboratories and quarantined 7 days prior to use. In addition, mice may be rendered neutropenic using cytoxan.

Infections:

Clinical isolates that have been adapted to cause infection in mice, are used in the experiment, including infections with strains of E. coli, K. pneumoniae, M. morganii, E. cloacae, S. marcescens, C. freundii, staphylococci, streptococci, P. aeruginosa and N. gonorrhoeae.

Preparation:

Animals are housed five to a cage with free access to food and water, in accordance with NIH guidelines.

Experimental Protocol:

Mice are challenged by injecting 0.5 ml intraperitoneally or 0.05 ml intranasally of a predetermined bacterial inoculum suspended in broth, saline or hog gastric mucin (supplemented with dried bovine hemoglobin for N. gonorrhoeae). The bacterial inoculum is equivalent to 10-100 LD₅₀s of the specific infecting strain and will result in death of the non-treated control animals within 7 days: “Bacterial Virulence in Mice”. Antibacterial doses (dose concentration prepared by two fold serial dilutions of the antibiotic) are dissolved or suspended in 0.2% aqueous agar or methocel, phosphate buffered saline or an adjuvant are administered orally, subcutaneously or intravenously in the following manner:

a) Orally or subcutaneously: Dose volume of 0.5 ml administered ½ hr after infection. A second dose may be administered 3 hr. after infection for treatment of infections with more virulent organisms.

b) Intravenously: Dose volume of 0.2 ml, administered ½ hr. after infection. For the treatment of infections with more virulent organisms, more doses, up to 48 hr may be administered. (Intravenous dosing will not exceed 3 doses/24 hr period.)

c) Oral pretreatment: Under special circumstances, the pH of the stomach needs to be adjusted in order to increase the gastric stability of the antibiotic. For this purpose, 0.5 ml of phosphate buffered saline (pH7.8, 0.06M) (or specific approved adjuvant) is administered orally ½ hr after infection, followed 5 minutes later by 0.5 ml of antibiotic (also orally) contained in phosphate buffered saline (pH7.8, 0.06M).

Animal Species

A detailed explanation as to the number of animals needed for the determination of in vivo efficacy follows:
A) Novel antibiotics are tested at 5 different dose levels with 5 mice per dose level at each of three routes of administration (oral, subcutaneous and intravenous). Initially the three routes of administration should be investigated so as to determine if the drug is orally absorbed and/or which is the most effective route. This would require 25 mice/route with 3 routes/antibiotic or 75 mice per novel compound tested. One to two novel antibiotics will be tested per experiment (75-150 mice)
B) The effectiveness of the new compound must be compared to that of a standard, or antibiotic of known effectiveness. Known or previously tested antibiotics are tested at 5 dose levels with 5 mice per dose level by a single route of administration, for a total of 25 mice/antibiotic. Usually 3-6 antibiotics will be tested per experiment. (75-150 mice).
C) Untreated controls—In each of the above tests, untreated animals are infected with 3 different concentrations of bacterial inoculum with 10 mice per concentration (30 mice total in each and every test). These untreated controls are used to determine and maintain the infection level between 10-100 LD50s as required for test to test comparison and validity.
   Determination of Protective Effects of Antibacterial Agents:

The protective effects of the antibacterial agent(s) are measured by the survival of the infected untreated as compared to the treated animals. For this determination, animals are observed for 7 days after treatment. A census of survivors is taken twice daily and at that time dead as well as moribund animals are removed. The 7 day survival ratio from three separate tests are pooled for estimation of median effective dose (ED50) by computerized program for probit analysis (Cleeland, R. and E. Squires. 1991. Evaluation of New Antimicrobials in Vitro and in Experimental Animal Infections. In Antibiotics in Laboratory Medicine”, 3rd. ed., edited by Victor Lorian. Willams and Wilkins Baltimore, Md. pp. 752-783). The test is performed three times on separate days to provide a statistically valid number of animals and to minimize variation in test results on a day to day and test to test basis.

Process of Invention

Compounds of the general formula I can be prepared by a reacting 6-methylidene penems of structure 2 (in the present invention, either bicyclic heteroaryl or tricyclic heteroaryl substituted on the methylidene linkage) (This class of compounds can be prepared by the procedure given in the application US. Application No. 60/377,052 and US. Application No. 60/377,051) (Scheme 1) with a proper nucleophile in an aprotic or a protic polar solvents (such as water, methanol, ethanol, THF, DMSO or DMF). The nucleophile can be but is not limited to a hydroxy anion, thiolate, alkoxy, aryloxy primary or a secondary amine. The NH in the thiazipine ring can be derivatized by a proper substituent during the course of these transformations. The chemistry involved in the deprotection step is well known to one of ordinary skill in the art.

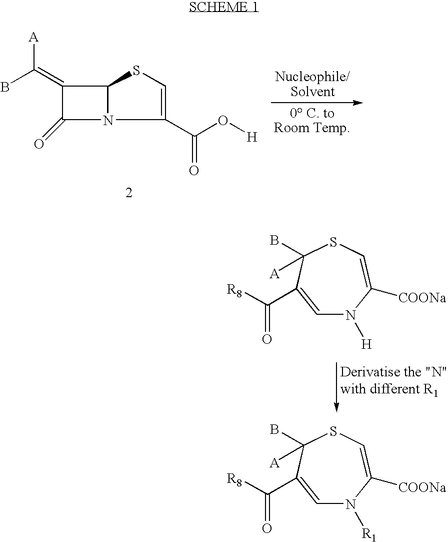

### EXPERIMENTALS

### GENERAL PROCEDURE TO PREPARE 7-BICYCLIC OR TRICYCLIC-4,7-DIHYDRO-[1,4]THIAZEPINE-3,6-DICARBOXYLIC ACID-6-METHYL ESTER

To a stirred solution of (5R)-Z-bicyclic or tricyclic heteroaryl-4-ylmethylene)penem-3-carboxylic acid (1.4 mmol) in anhydrous methanol (50 ml) at 0° C., NaOMe (80.0 mg 1.5 mmol) is added. The reaction mixture is stirred at room temperature for 2 hrs and concentrated. The product is purified by HP21 resin reverse phase column chromatography. Initially the column is eluted with deionized water (2 L) and later with 10% acetonitrile:water. The fractions containing the product are collected and concentrated under reduced pressure at room temperature. The yellow solid is washed with acetone, filtered and dried.

### Example 1

### PREPARATION OF 7-(5,6-DIHYDRO-8H-IMIDAZO[2,1-C][1,4]OXAZIN-2-YL)-4,7-DIHYDRO-[1,4]THIAZEPINE-3,6-DICARBOXYLIC ACID-6-METHYL ESTER-3-SODIUM SALT

7-(5,6-dihydro-8H-imidazo[2,1-c][1,4]oxazin-2-yl)-4,7-dihydro-[1,4]thiazepine-3,6-dicarboxylic acid-6-methyl ester-3-sodium salt is prepared by following the general procedure as outlined above. Starting from (5R) (6Z)-6-(5,6-dihydro-8H-imidazo[2,1-c][1,4]oxazin-2-ylmethylene)-7-oxo-4-thia-1-azabicyclo[3.2.0]hept-2-ene-2-carboxylic acid sodium salt (500 mg, 1.4 mmol) and sodium methoxide (80 mg, 1.5 mmol) the titled compound is prepared. Yellow amorphous solid; Yield 387 mg, 70%; Mp: 225° C. (Dec); ¹H NMR: (DMSO-d₆) δ 9.16 (d, 1H), 7.64 (d, 1H), 6.24 (s, 1H), 6.08 (s, 1H), 5.41 (s, 1H), 4.64 (q, 2H), 3.98-3.80 (m, 4H), 3.54 (s, 3H); (M+H+Na) 360.

### Example 2

### PREPARATION OF 7-(5,6,7,8-TETRAHYDRO-8H-IMIDAZO[1,2-A][1,4]PYRAZIN2-YL)-4,7-DIHYDRO-[1,4]THIAZEPINE-3,6-DICARBOXYLIC ACID-3, 6-DISODIUM SALT

To a stirred solution of (5R), (6Z)-7-Oxo-6-(5,6,7,8-tetrahydroimidazo[1,2-a]pyrazin-2-ylmethylene)-4-thia-1-azabicyclo[3.2.0]hept-2-ene-2-carboxylic acid, sodium salt (326 mg, 1 mmol) 0.1 N NaOH (15 ml) is added at room temperature. The reaction mixture is stirred for 1 hr and concentrated. The red solid obtained is dissolved in water and loaded on a HP21 resin reverse phase column chromatography. Initially the column was eluted with deionized water (2 L) and latter with 5% acetonitrile:water. The fractions containing the product are collected and concentrated under reduced pressure at room temperature. The red solid is washed with acetone, filtered and dried. Red amorphous solid; Yield 200 mg, 54%; Mp: 225° C. (Dec); ¹H NMR: (DMSO-d₆) δ 9.31 (s, 1H), 7.82 (s, 1H), 6.66 (s, 1H), 6.10 (s, 1H), 3.95-3.78 (m, 3H), 2.9-3.1 (m, 4H); (M+H) 324.

What is claimed is:

1. A method of treating a bacterial infection which comprises providing a beta-lactam antibiotic and a compound of formula I or a pharmaceutically acceptable salt thereof: 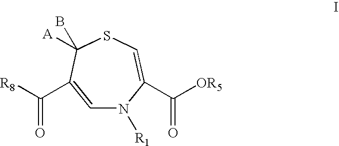
Wherein:
one of A and B denotes hydrogen and the other is a fused bicyclic heteroaryl group or a fused tricyclic heteroaryl group;
R₁ is H, optionally substituted —C1-C6 alkyl, optionally substituted -aryl, optionally substituted -heteroaryl or mono or bicyclic saturated heterocycles, optionally substituted —C3-C7 cycloalkyl, optionally substituted —C3-C6 alkenyl, optionally substituted —C3-C6 alkynyl with the proviso that both the double bond and the triple bond should not be present at the carbon atom which is directly linked to N; optionally substituted —C1-C6 per fluoro alkyl, —S(O)ₚ optionally substituted alkyl or aryl where p is 2, optionally substituted —C═Oheteroaryl, optionally substituted —C═Oaryl, optionally substituted —C═O(C1-C6) alkyl, optionally substituted —C═O(C3-C6) cycloalkyl, optionally substituted —C═O mono or bicyclic saturated heterocycles, optionally substituted C1-C6 alkyl aryl, optionally substituted C1-C6 alkyl heteroaryl, optionally substituted aryl-C1-C6 alkyl, optionally substituted heteroaryl-C1-C6 alkyl, optionally substituted C1-C6 alkyl mono or bicyclic saturated heterocycles, optionally substituted arylalkenyl of 8 to 16 carbon atoms, —CONR₆R₇, —SO₂NR₆R₇, optionally substituted arylalkyloxyalkyl, optionally substituted -alkyl-O-alkyl-aryl, optionally substituted -alkyl-O-alkyl-heteroaryl, optionally substituted aryloxyalkyl, optionally substituted heteroaryloxyalkyl, optionally substituted aryloxyaryl, optionally substituted aryloxyheteroaryl, optionally substituted C1-C6alkyl aryloxyaryl, optionally substituted C1-C6 alkyl aryloxyheteroaryl, optionally substituted alkyl aryloxy alkylamines, optionally substituted alkoxy carbonyl, optionally substituted aryloxy carbonyl, or optionally substituted heteroaryloxy carbonyl;
R₃ is hydrogen, C1-C6 alkyl, C3-C6 cycloalkyl, optionally substituted aryl, or optionally substituted heteroaryl;
R₅ is H, C1-C6 alkyl, C5-C6 cycloalkyl, or salts selected from the group Na, K, and Ca;
R₆ and R₇ are independently H, optionally substituted C1-C6 alkyl, optionally substituted aryl, optionally substituted heteroaryl, optionally substituted C1-C6 alkyl aryl, optionally substituted arylalkyl, optionally substituted heteroarylalkyl, optionally substituted C1-C6 alkyl heteroaryl, R₆ and R₇ can be together to form a 3-7 membered saturated ring system optionally having one or two heteroatoms selected from the group N—R₁, O, and S═(O)ₙ;
n=0-2; and
R₈ is N—R₆R₇, optionally substituted C1-C6 alkoxy, hydroxy, optionally substituted —S—C1-C6 alkyl, optionally substituted —O-Aryl, optionally substituted —O—C1-C6-alkyl-aryl, optionally substituted —O-aryl alkyl(C1-C6), optionally substituted —S-Aryl, optionally substituted —S—C1-C6-alkyl-aryl, optionally substituted —S-aryl-alkyl(C1-C6), —S— optionally substituted C1-C6 alkyl-COO—H, or —S— optionally substituted C1-C6 alkyl-COO—C1-C6 alkyl.

2. The method according to claim 1 wherein the fused bicyclic heteroaryl group has the structural formula 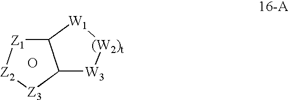
wherein Z₁, Z₂, and Z₃ are independently CR₂, N, O, S or N—R₁, provided one of Z₁, Z₂, or Z₃ is carbon and is bonded to the remainder of the molecule as shown in formula I;
W₁, W₂ and W₃ are independently CR₄R₄, S, SO, SO₂, O, N—R₁, C═O; with the proviso that no S—S or O—O or S—O bond formation can occur to form the saturated ring system;
t=1 to 4;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine; and
R₄ is H, optionally substituted C1-C6 alkyl, one of R₄ is OH, C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S═(O)ₙ (where n=0 to 2), N—R₁.

3. The method according to claim 1 wherein the fused bicyclic heteroaryl group has the structural formula
wherein 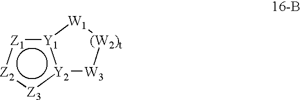
Z₁, Z₂ and Z₃ are independently CR₂, N, O, S or N—R₁ provided one of Z₁-Z₃ is carbon and is bonded to the remainder of the molecule;
W₁, W₂ and W₃ are independently CR₄R₄, S, SO, SO₂, O, or N—R₁;
t=1 to 4;
Y₁ and Y₂ are independently N or C; with the proviso that if the aromatic ring portion of the bicyclic heteroaryl group is imidazole, the nonaromatic ring portion may not contain a S adjacent to the bridgehead carbon;
R₁, R₆ and R₇ are as defined in claim 1;
R₄ is H, optionally substituted C1-C6 alkyl, one of R₄ is OH, C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S═(O)ₙ (where n=0 to 2), N—R₁; and
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine.

4. The method according to claim 1 wherein the fused bicyclic heteroaryl group is 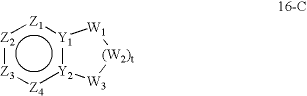
wherein
Z₁, Z₂, Z₃, and Z₄ are independently CR₂ or N provided one of Z₁-Z₄ is carbon and is bonded to the remainder of the molecule;
W₁, W₂ and W₃ are independently CR₄R₄, S, SO, SO₂, O, or N—R₁; with the proviso that no S—S or O—O or S—O bond formation can occur to form the saturated ring system;
t=1 to 4;
Y₁ and Y₂ are independently C or N;
R₄ is H, optionally substituted C1-C6 alkyl, one of R₄ is OH, C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S═(O)ₙ (where n=0 to 2), N—R₁;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine; and
R₁, R₆, and R₇ are as defined in claim 1.

5. The method according to claim 1 wherein the fused tricyclic heteroaryl group has the formula 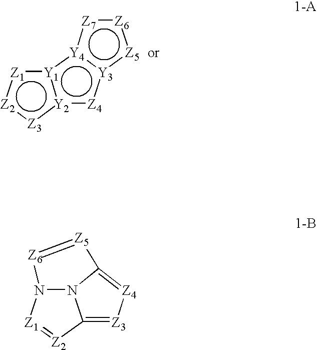
wherein Z₁, Z₂, Z₃, Z₄, Z₅, Z₆ and Z₇ are independently CR₂, N, O, S or N—R₁, provided one of Z₁-Z₇ is a carbon atom to which the remainder of the molecule is attached;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
and Y₁, Y₂, Y₃ and Y₄ may independently be C or N.

6. The method according to claim 1 wherein the tricyclic heteroaryl group is 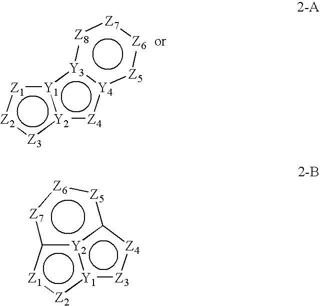
wherein Z₁, Z₂, Z₃, Z₄, Z₅, Z₆, Z₇ and Z₈ are independently CR₂, N, O, S or N—R₁ provided one of the Z₁-Z₈ is a carbon atom to which the remainder of the molecule is attached;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
and Y₁, Y₂, Y₃ and Y₄ are independently C or N.

7. The method according to claim 1 wherein the tricyclic heteroaryl group is 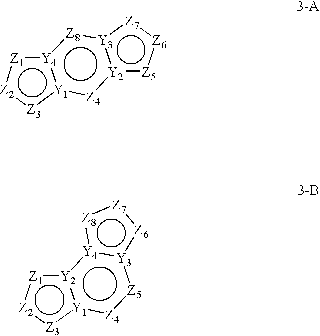
wherein Z₁, Z₂, Z₃, Z₄, Z₅, Z₆, Z₇ and Z₈ are independently CR₂, N, O, S or N—R₁ provided one of Z₁-Z₈ is a carbon atom to which the remainder of the molecule is attached;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
and Y₁, Y₂, Y₃ and Y₄ may be C or N.

8. The method according to claim 1 wherein the tricyclic heteroaryl group is 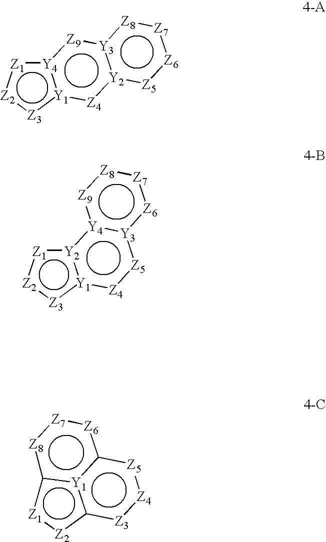
wherein Z₁, Z₂, Z₃, Z₄, Z₅, Z₆, Z₇, Z₈ and Z₉ are independently CR₂, N, O, S or N—R₁ provided one of the Z₁-Z₉ is a carbon atom to which the remainder of the molecule is attached;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine; and
Y₁, Y₂, Y₃ and Y₄ are independently C or N.

9. The method according to claim 1 wherein the tricyclic heteroaryl group is 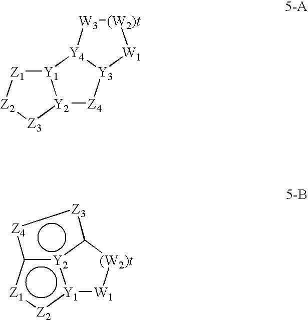
wherein Z₁, Z₂, Z₃ and Z₄ are independently CR₂, N, O, S or N—R₁, provided one of Z₁-Z₄ is a carbon atom to which the remainder of the molecule is attached;
Y₁, Y₂, Y₃ and Y₄ are independently C or N;
W₁, W₂ and W₃ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
and t=1 to 3.

10. The method according to claim 1 wherein the tricyclic heteroaryl group is 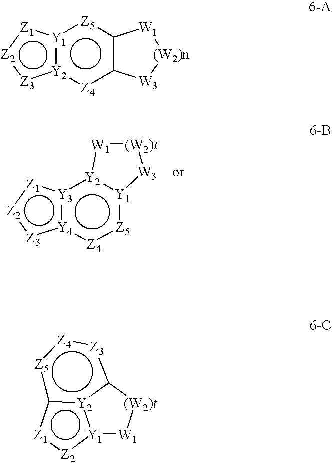
wherein Z₁, Z₂, Z₃, Z₄ and Z₅ are independently CR₂, N, O, S or N—R₁, provided one of Z₁-Z₅ is a carbon atom to which the remainder of the molecule is attached;
Y₁, and Y₂ are independently C or N;
W₁, W₂ and W₃ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁₁;
and t=1 to 3.

11. The method according to claim 1 wherein the tricyclic heteroaryl group is 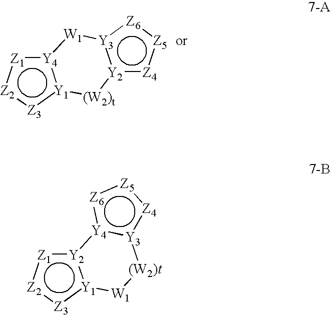
wherein Z₁, Z₂, Z₃, Z₄, Z₅ and Z₆ are independently CR₂, N, O, S, and N—R₁; provided one of Z₁-Z₆ is a carbon atom to which the remainder of the molecule is attached; Y₁, Y₂, Y₃ and Y₄ are independently C or N;
W₁ and W₂ are independently CR₄R₄, S(O)r (r=0-2), O, N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
and t=1 to 3.

12. The method according to claim 1 wherein the tricyclic heteroaryl group is 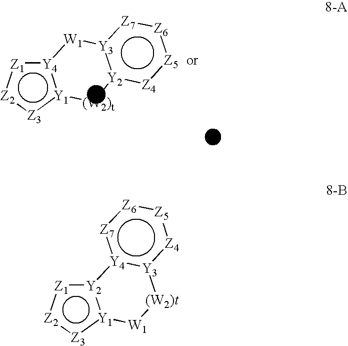
wherein Z₁, Z₂, Z₃, Z₄, Z₅, Z₆ and Z₇ are independently CR₂, N, O, S or N—R₁, provided one of the Z₁-Z₇ is a carbon atom to which the remainder of the molecule is attached;
Y₁, Y₂, Y₃ and Y₄ are independently C or N;
W₁ and W₂ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
and t=0-3.

13. The method according to claim 1 wherein the tricyclic heteroaryl group is 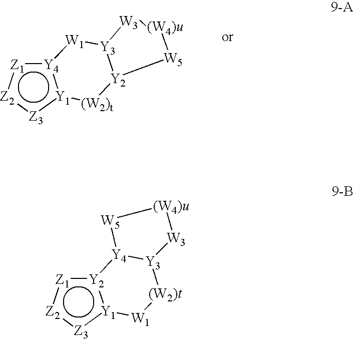
wherein Z₁, Z₂ and Z₃ are independently CR₂, N, O, S or N—R₁, provided one of Z₁-Z₃ is a carbon atom to which the remainder of the molecule is attached;
Y₁ and Y₄ are independently C or N;
Y₂ and Y₃ are independently CH or N;
W₁, W₂, W₃, W₄ and W₅ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
t=0 to 2; and
u=1 to 3.

14. The method according to claim 1 wherein the tricyclic heteroaryl group is 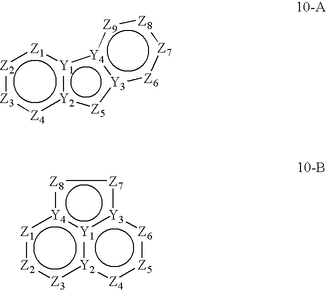
wherein Z₁, Z₂, Z₃, Z₄, Z₅, Z₆, Z₇, Z₈ and Z₉ are independently CR₂, N, O, S or N—R₁ provided one of the Z₁-Z₉ is a carbon atom to which the remainder of the molecule is attached; R₁, R₆ and R₇ are as defined in claim 1; R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
and Y₁, Y₂, Y₃ and Y₄ are independently C or N.

15. The method according to claim 1 wherein the tricyclic heteroaryl group is 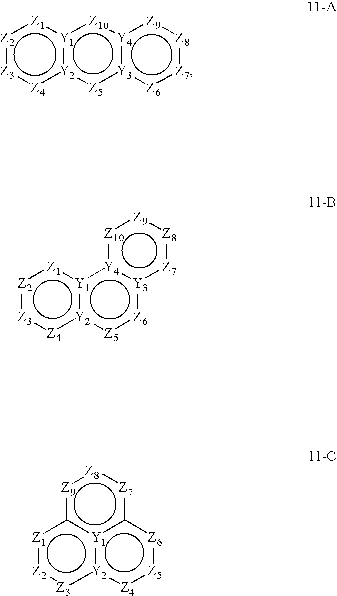
wherein Z₁, Z₂, Z₃, Z₄, Z₅, Z₆, Z₇, Z₈, Z₉ and Z₁₀ are independently CR₂, N, O, S or N—R₁, provided one of Z₁-Z₁₀ is a carbon atom to which the remainder of the molecule is attached;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine; and
Y₁, Y₂, Y₃ and Y₄ are independently C or N.

16. The method according to claim 1 wherein the tricyclic heteroaryl group is 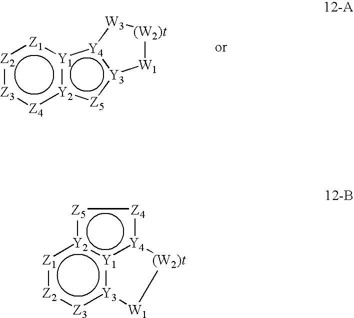
wherein Z₁, Z₂, Z₃, Z₄ and Z₅ are independently CR₂, N, O, S or N—R₁, provided that one of Z₁-Z₅ is a carbon atom to which the remainder of the molecule is attached;
Y₁, Y₂, Y₃ and Y₄ are independently C or N;
W₁, W₂, W₃ are independently CR₄R₄ O, N—R₁, or S═(O)ᵣ (r=0-2) with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
and t=1-4.

17. The method according to claim 1 wherein the tricyclic heteroaryl group is 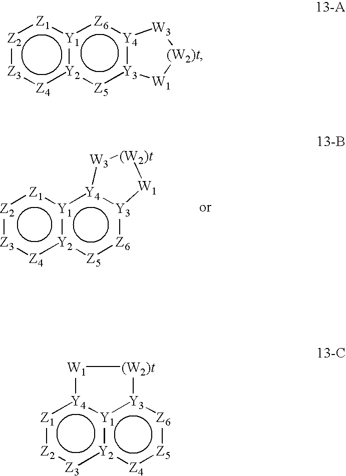
wherein Z₁, Z₂, Z₃, Z₄, Z₅ and Z₆ are independently CR₂, N, O, S or N—R₁, provided one of Z₁-Z₆ is a carbon atom to which the remainder of the molecule is attached;
Y₁, Y₂, Y₃ and Y₄ are independently C or N;
W₁, W₂ and W₃ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
and t=1 to 3.

18. The method according to claim 1 wherein the tricyclic heteroaryl group is 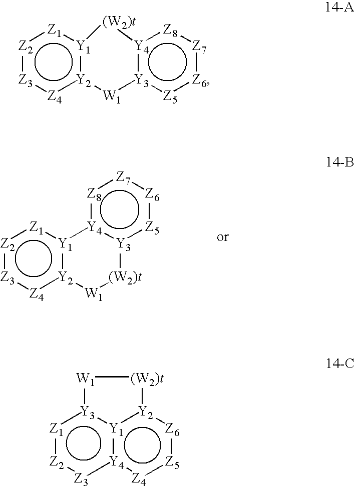
wherein Z₁, Z₂, Z₃, Z₄, Z₅, Z₆, Z₇ and Z₈ are independently CR₂, N, O, S or N—R₁ provided one of Z₁-Z₈ is a carbon atom to which the remainder of the molecule is attached;
Y₁, Y₂, Y₃ and Y₄ are independently C or N;
W₁, and W₂ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O), (where n=0 to 2), N—R₁;
and t=1 to 2.

19. The method according to claim 1 wherein the tricyclic heteroaryl group is 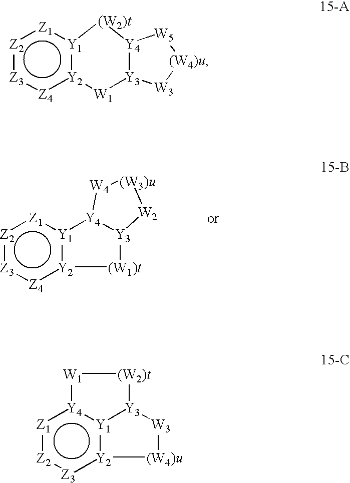
wherein Z₁, Z₂, Z₃ and Z₄ are independently CR₂, N, O, S or N—R₁, provided one of Z₁-Z₄ is a carbon atom to which the remainder of the molecule is attached;
Y₁, Y₂, Y₃ and Y₄ are independently C or N;
W₁, W₂, W₃, W₄ and W₅ are independently CR₄R₄, S(O)r (r=0-2), O, or N—R₁ with the proviso that no S—S, S—O or O—O bond formation can occur to form a saturated ring;
R₁, R₆ and R₇ are as defined in claim 1;
R₂ is hydrogen, optionally substituted C1-C6 alkyl, optionally substituted C2-C6 alkenyl, optionally substituted C2-C6 alkynyl, halogen, cyano, N—R₆R₇, optionally substituted C1-C6 alkoxy, hydroxy; optionally substituted aryl, optionally substituted heteroaryl, COOR₆, optionally substituted alkylaryloxyalkylamines, optionally substituted aryloxy, optionally substituted heteroaryloxy, optionally substituted C3-C6 alkenyloxy, optionally substituted C3-C6 alkynyloxy, C1-C6 alkylamino-C1-C6 alkoxy, alkylenedioxy, optionally substituted aryloxy-C1-C6 alkyl amine, C1-C6 perfluoro alkyl, S(O)_{q}-optionally substituted C1-C6 alkyl, S(O)_{q}— optionally substituted aryl where q is 0, 1 or 2, CONR₆R₇, guanidino or cyclic guanidino, optionally substituted alkylaryl, optionally substituted arylalkyl, optionally substituted C1-C6 alkylheteroaryl, optionally substituted heteroaryl-C1-C6 alkyl, optionally substituted C1-C6 alkyl mono or bicyclic saturated heterocycles, optionally substituted arylalkenyl of 8 to 16 carbon atoms, SO₂NR₆R₇, optionally substituted arylalkyloxyalkyl, optionally substituted aryloxyalkyl, optionally substituted heteroaryloxyalkyl, optionally substituted aryloxyaryl, optionally substituted aryloxyheteroaryl, optionally substituted heteroaryloxyaryl, optionally substituted C1-C6alkyl aryloxyaryl, optionally substituted C1-C6 alkylaryloxyheteroaryl, optionally substituted aryloxyalkyl, optionally substituted heteroaryloxyalkyl, or optionally substituted alkylaryloxyalkylamine;
R₄ is H, optionally substituted C1-C6 alkyl, OH (provided both R₄ are not OH), C1-C6 alkoxy, —S—C1-C6 alkyl, COOR₆, —NR₆R₇, —CONR₆R₇; or R₄R₄ may together be ═O or R₄R₄ together with the carbon to which they are attached may form a spiro system of five to eight members with or without the presence of heteroatoms selected N, O, S(O)ₙ (where n=0 to 2), N—R₁;
t=1 to 3; and
u=1 to 3.

20. The method according to claim 1 wherein the beta-lactam antibiotic is a penicillin, a cephalosporin, aztreonam or latamoxef.

21. The method according to claim 20 wherein the beta-lactam antibiotic is a penicillin antibiotic.

22. The method according to claim 21 wherein the penicillin antibiotic is piperacillin or amoxycillin.

23. A pharmaceutical composition comprising an effective amount of beta-lactam antibiotic, a pharmaceutically acceptable carrier and an effective amount of a compound of formula 1 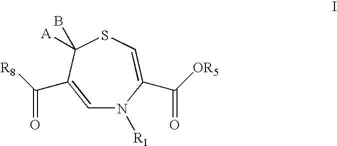
wherein:
one of A and B denotes hydrogen and the other is a fused bicyclic heteroaryl group or a fused tricyclic heteroaryl group;
R₁ is H, optionally substituted —C1-C6 alkyl, optionally substituted -aryl, optionally substituted -heteroaryl or mono or bicyclic saturated heterocycles, optionally substituted —C3-C7 cycloalkyl, optionally substituted —C3-C6 alkenyl, optionally substituted —C3-C6 alkynyl with the proviso that both the double bond and the triple bond should not be present at the carbon atom which is directly linked to N; optionally substituted —C1-C6 per fluoro alkyl, —S(O)ₚ optionally substituted alkyl or aryl where p is 2, optionally substituted —C═Oheteroaryl, optionally substituted —C═Oaryl, optionally substituted —C═O(C1-C6) alkyl, optionally substituted —C═O(C3-C6) cycloalkyl, optionally substituted —C═O mono or bicyclic saturated heterocycles, optionally substituted C1-C6 alkyl aryl, optionally substituted C1-C6 alkyl heteroaryl, optionally substituted aryl-C1-C6 alkyl, optionally substituted heteroaryl-C1-C6 alkyl, optionally substituted C1-C6 alkyl mono or bicyclic saturated heterocycles, optionally substituted arylalkenyl of 8 to 16 carbon atoms, —CONR₆R₇, —SO₂NR₆R₇, optionally substituted arylalkyloxyalkyl, optionally substituted -alkyl-O-alkyl-aryl, optionally substituted -alkyl-O-alkyl-heteroaryl, optionally substituted aryloxyalkyl, optionally substituted heteroaryloxyalkyl, optionally substituted aryloxyaryl, optionally substituted aryloxyheteroaryl, optionally substituted C1-C6alkyl aryloxyaryl, optionally substituted C1-C6 alkyl aryloxyheteroaryl, optionally substituted alkyl aryloxy alkylamines, optionally substituted alkoxy carbonyl, optionally substituted aryloxy carbonyl, or optionally substituted heteroaryloxy carbonyl;
R₃ is hydrogen, C1-C6 alkyl, C3-C6 cycloalkyl, optionally substituted aryl, or optionally substituted heteroaryl;
R₅ is H, C1-C6 alkyl, C5-C6 cycloalkyl, or salts selected from the group Na, K, and Ca;
R₆ and R₇ are independently H, optionally substituted C1-C6 alkyl, optionally substituted aryl, optionally substituted heteroaryl, optionally substituted C1-C6 alkyl aryl, optionally substituted arylalkyl, optionally substituted heteroarylalkyl, optionally substituted C1-C6 alkyl heteroaryl, R₆ and R₇ can be together to form a 3-7 membered saturated ring system optionally having one or two heteroatoms selected from the group N—R₁, O, and S═(O)ₙ;
n=0-2; and
R₈ is N—R₆R₇, optionally substituted C1-C6 alkoxy, hydroxy, optionally substituted —S—C1-C6 alkyl, optionally substituted —O-Aryl, optionally substituted —O—C1-C6-alkyl-aryl, optionally substituted —O-aryl alkyl(C1-C6), optionally substituted —S-Aryl, optionally substituted —S—C1-C6-alkyl-aryl, optionally substituted —S-aryl-alkyl(C1-C6), —S— optionally substituted C1-C6 alkyl-COO—H, or —S— optionally substituted C1-C6 alkyl-COO—C1-C6 alkyl.

24. The composition of claim 23 wherein the beta-lactam antibiotic is piperacillin or amoxycillin.
